(19) United States
(12) Patent Application Publication
(10) Pub. No.: US 2019/0032150 A1
(43) Pub. Date: Jan. 31, 2019

(54) DIAGNOSTIC AND THERAPEUTIC METHODS FOR CANCER

(71) Applicant: Genentech, Inc., South San Francisco, CA (US)

(72) Inventors: Daniel Christopher Kirouac, Alameda, CA (US); Marie-Claire Wagle, Burlingame, CA (US); Shih-Min Huang, Oakland, CA (US)

(21) Appl. No.: 16/157,582

(22) Filed: Oct. 11, 2018

Related U.S. Application Data
(63) Continuation of application No. PCT/US2017/026941, filed on Apr. 11, 2017.
(60) Provisional application No. 62/323,210, filed on Apr. 15, 2016.

(51) Int. Cl. C12Q 1/6886; A61K 31/4523; A61K 31/437; A61K 45/06

(52) U.S. Cl. CPC C12Q 1/6886; A61K 31/4523; C12Q 2600/158; A61K 45/06; C12Q 2600/106; A61K 31/437

(57) ABSTRACT

The present invention provides diagnostic and therapeutic methods and compositions for cancer. The invention provides methods of determining whether a patient having a cancer is likely to respond to treatment comprising a MAPK signaling inhibitor, methods of predicting responsiveness of a patient having a cancer to treatment comprising one or more MAPK signaling inhibitors, methods of selecting a therapy for a patient having a cancer, and methods of treating cancer based on expression levels of biomarkers of the invention (e.g., the expression levels of DUSP6, ETV4, SPRY2, SPRY4, PHLDA1, ETV5, DUSP4, CCND1, EPHA2, and EPHA4).

### SEQUENCE LISTING

The instant application contains a Sequence Listing which has been submitted electronically in ASCII format and is hereby incorporated by reference in its entirety. Said ASCII copy, created on Oct. 10, 2018, is named 50474-133002_Sequence_Listing_10.10.18_ST25 and is 100,614 bytes in size.

### FIELD OF THE INVENTION

The present invention is directed to diagnostic and therapeutic methods for the treatment of proliferative cell disorders (e.g., cancers) using MAPK (e.g., mitogen-activated protein kinase) signaling inhibitors. Also provided are related kits and compositions.

### BACKGROUND

Cancer remains one of the most deadly threats to human health. Certain cancers can metastasize and grow rapidly in an uncontrolled manner, making timely detection and treatment extremely difficult. In the U.S., cancer affects nearly 1.3 million new patients each year and is the second leading cause of death after heart disease, accounting for approximately one in four deaths. The mitogen-activated protein kinase (MAPK) signaling pathway is activated in more than 30% of human cancers, most commonly in the MEK/ERK arm of the pathway via mutations in KRAS and/or in BRAF. RAS mutations occur with a frequency of 90% in pancreatic tumors, 35% in lung adenocarcinoma (non-small cell lung cancer (NSCLC)) tumors, 45% in colorectal tumors, and 15% in melanoma tumors. BRAF mutations occur in 66% of melanoma tumors and 12% of colorectal tumors. Tumors with KRAS mutations were predicted to be sensitive to MEK inhibition due to activation of MAPK signaling. However, MEK inhibitors in multiple clinical trials, either as a monotherapy or in combination with chemotherapies, have not shown superior efficacy in the KRAS mutant subgroup compared to the KRAS wild-type subgroup, indicating a limitation of utilizing KRAS mutation status as a predictive biomarker of MEK inhibitor sensitivity. In addition, stratification based on KRAS mutation status may inadvertently overlook wild-type KRAS tumors that could be addicted to MAPK signaling, independent of KRAS mutation status.

Thus, there remains a need to develop improved alternative methods for diagnosing and treating patient populations best suited for treatment including one or more MAPK signaling inhibitors.

### SUMMARY OF THE INVENTION

The present invention provides diagnostic and therapeutic methods, kits, and compositions for the treatment of proliferative cell disorders (e.g., cancers).

In a first aspect, the invention features a method of identifying a patient having a cancer who may benefit from treatment comprising one or more MAPK (mitogen-activated protein kinase) signaling inhibitors, the method comprising determining an expression level of at least one gene (e.g., one, two, three, four, five, six, seven, eight, nine, or ten genes) selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level identifies the patient as one who may benefit from treatment comprising one or more MAPK signaling inhibitors.

In a second aspect, the invention features a method of optimizing therapeutic efficacy for treatment of a patient having a cancer, the method comprising determining an expression level of at least one gene (e.g., one, two, three, four, five, six, seven, eight, nine, or ten genes) selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment comprising one or more MAPK signaling inhibitors.

In a third aspect, the invention features a method of predicting responsiveness of a patient having a cancer to treatment comprising one or more MAPK signaling inhibitors, the method comprising determining an expression level of at least one gene (e.g., one, two, three, four, five, six, seven, eight, nine, or ten genes) selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment comprising one or more MAPK signaling inhibitors.

In a fourth aspect, the invention features a method of selecting a treatment for a patient having a cancer, the method comprising determining an expression level of at least one gene (e.g., one, two, three, four, five, six, seven, eight, nine, or ten genes) selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment comprising one or more MAPK signaling inhibitors.

In some embodiments of any one of the first, second, third, and fourth aspects, the method comprises determining the expression levels of at least four genes (e.g., four, five, six, seven, eight, nine, or ten genes) selected from DUSP6, ETV4, SPRY2, SPRY4, PHLDA1, ETV5, DUSP4, CCND1, EPHA2, and EPHA4. In some embodiments, the at least four genes comprise DUSP6, ETV4, SPRY2, and SPRY4. In other embodiments, the method comprises determining the expression levels of at least five genes selected from DUSP6, ETV4, SPRY2, SPRY4, PHLDA1, ETV5, DUSP4, CCND1, EPHA2, and EPHA4. In some embodiments, the at least five genes comprise DUSP6, ETV4, SPRY2, SPRY4, and PHLDA1. In other embodiments, the method comprises determining the expression levels of at least six genes selected from DUSP6, ETV4, SPRY2, SPRY4, PHLDA1, ETV5, DUSP4, CCND1, EPHA2, and EPHA4. In some embodiments, the at least six genes comprise DUSP6, ETV4, SPRY2, SPRY4, PHLDA1, and ETV5. In other embodiments, the method comprises determining the expression levels of at least seven genes selected from DUSP6, ETV4, SPRY2, SPRY4, PHLDA1, ETV5, DUSP4, CCND1, EPHA2, and EPHA4. In some embodiments, the at least seven genes comprise DUSP6, ETV4, SPRY2, SPRY4, PHLDA1, ETV5, and DUSP4. In other embodiments, the method comprises determining the expression levels of at least eight genes selected from DUSP6, ETV4, SPRY2, SPRY4, PHLDA1, ETV5, DUSP4, CCND1, EPHA2, and EPHA4. In some embodiments, the at least eight genes comprise DUSP6, ETV4, SPRY2, SPRY4, PHLDA1, ETV5, DUSP4, and CCND1. In other embodiments, the method comprises determining the expression levels of at least nine genes selected from DUSP6, ETV4, SPRY2, SPRY4, PHLDA1, ETV5, DUSP4, CCND1, EPHA2, and EPHA4. In some embodiments, the at least nine genes comprise DUSP6, ETV4, SPRY2, SPRY4, PHLDA1, ETV5, DUSP4, CCND1, and EPHA2. In other embodiments, the method comprises determining the expression levels of DUSP6, ETV4, SPRY2, SPRY4, PHLDA1, ETV5, DUSP4, CCND1, EPHA2, and EPHA4.

In other embodiments of any one of the first, second, third, and fourth aspects, method further comprises determining a MAPK activity score, wherein the MAPK activity score is determined according to the algorithm:

$$\frac{Σ {z}_{i}}{\sqrt{n}} ,$$

where zᵢ is the z-score of each gene, normalized across all samples or to a set of housekeeping genes, and n is the number of genes comprising the set. In some embodiments, a MAPK activity score greater than a median MAPK activity score is a high MAPK activity score and identifies a patient who has an increased likelihood of benefiting from treatment comprising one or more MAPK signaling inhibitors. In other embodiments, a MAPK activity score less than a median MAPK activity score is a low MAPK activity score and identifies a patient who has a decreased likelihood of benefiting from treatment comprising one or more MAPK signaling inhibitors. In some embodiments, the patient has a high MAPK activity score and the method further comprises administering to the patient a therapeutically effective amount of one or more MAPK signaling inhibitors. In some embodiments, the administering of the one or more MAPK signaling inhibitors is after the determining of the expression level of the at least one gene. In some embodiments, the administering of the one or more MAPK signaling inhibitors is before the determining of the expression level of the at least one gene.

In a fifth aspect, the invention features a method of treating a patient having a cancer, comprising administering to the patient a therapeutically effective amount of one or more MAPK signaling inhibitors, wherein the expression level of at least one gene (e.g., one, two, three, four, five, six, seven, eight, nine, or ten genes) selected from the group consisting of DUSP6, ETV4, SPRY2, SPRY4, PHLDA1, ETV5, DUSP4, CCND1, EPHA2, and EPHA4 in a sample obtained from the patient have been determined to be increased as compared to a reference level. In some embodiments, the expression levels of at least four genes have been determined to be increased in the patient sample relative to a reference level. In some embodiments, the expression levels of DUSP6, ETV4, SPRY2, and SPRY4 have been determined to be increased in the patient sample relative to a reference level. In other embodiments, the expression levels of at least five genes have been determined to be increased in the patient sample relative to a reference level. In some embodiments, the expression levels of DUSP6, ETV4, SPRY2, SPRY4, and PHLDA1 have been determined to be increased in the patient sample relative to a reference level. In other embodiments, the expression levels of at least six genes have been determined to be increased in the patient sample relative to a reference level. In some embodiments, the expression levels of DUSP6, ETV4, SPRY2, SPRY4, PHLDA1, and ETV5 have been determined to be increased in the patient sample relative to a reference level. In other embodiments, the expression levels of at least seven genes have been determined to be increased in the patient sample relative to a reference level. In some embodiments, the expression levels of DUSP6, ETV4, SPRY2, SPRY4, PHLDA1, ETV5, and DUSP4 have been determined to be increased in the patient sample relative to a reference level. In other embodiments, the expression levels of at least eight genes have been determined to be increased in the patient sample relative to a reference level. In some embodiments, the expression levels of DUSP6, ETV4, SPRY2, SPRY4, PHLDA1, ETV5, DUSP4, and CCND1 are determined to be increased in the patient sample relative to a reference level. In other embodiments, the expression levels of at least nine genes have been determined to be increased in the patient sample relative to a reference level. In some embodiments, the expression levels of DUSP6, ETV4, SPRY2, SPRY4, PHLDA1, ETV5, DUSP4, CCND1, and EPHA2 have been determined to be increased in the patient sample relative to a reference level. In other embodiments, the expression levels of DUSP6, ETV4, SPRY2, SPRY4, PHLDA1, ETV5, DUSP4, CCND1, EPHA2, and EPHA4 have been determined to be increased in the patient sample relative to a reference level.

In some embodiments of the fifth aspect, a high MAPK activity score has been determined for the patient according to the algorithm:

$$\frac{Σ {z}_{i}}{\sqrt{n}} ,$$

where zᵢ is the z-score of each gene, normalized across all samples or to a set of housekeeping genes, and n is the number of genes comprising the set, wherein the high MAPK activity score is greater than a median MAPK activity score and identifies a patient who has an increased likelihood of benefiting from treatment comprising one or more MAPK signaling inhibitors.

In some embodiments of any one of the first, second, third, fourth, and fifth aspects, the median MAPK activity score is a previously defined median MAPK activity score for the cancer. In some embodiments, the previously defined median MAPK activity score is determined from a plurality of samples (e.g., archived samples) from patients having the cancer. In other embodiments, the sample obtained from the patient is a tissue sample, a whole blood sample, a plasma sample, or a serum sample. In some embodiments, the tissue sample is a tumor tissue sample. In other embodiments, the expression level is an mRNA expression level. In some embodiments, the mRNA expression level is determined by RNA-Seq, PCR, RT-PCR, gene expression profiling, serial analysis of gene expression, microarray analysis, or whole genome sequencing. In some embodiments, the mRNA expression level is determined by RNA-Seq. In other embodiments, the expression level is a protein expression level.

In other embodiments of any one of the first, second, third, fourth, and fifth aspects, the cancer is selected from the group consisting of a lung cancer, breast cancer, skin cancer, colorectal cancer, stomach cancer, lymphoid cancer, ovarian cancer, cervical cancer, peritoneal cancer, pancreatic cancer, glioblastoma, liver cancer, bladder cancer, colon cancer, rectal cancer, endometrial cancer, uterine cancer, salivary gland cancer, renal cancer, prostate cancer, vulval cancer, thyroid cancer, anal cancer, penile cancer, and head and neck cancer. In some embodiments, the cancer is a lung cancer, breast cancer, skin cancer, colorectal cancer, or stomach cancer. In some embodiments, the cancer is a lung cancer. In some embodiments, the lung cancer is non-small cell lung cancer (NSCLC). In some embodiments, the cancer is a skin cancer. In some embodiments, the skin cancer is a melanoma. In some embodiments, the melanoma is a metastatic melanoma. In some embodiments, the melanoma is a locally advanced melanoma. In some embodiments, the metastatic melanoma or locally advanced melanoma is an unresectable melanoma.

In some embodiments of any one of the first, second, third, fourth, and fifth aspects, the one or more MAPK signaling inhibitors are selected from the group consisting of a MEK inhibitor, an ERK inhibitor, a BRAF inhibitor, a CRAF inhibitor, a RAF inhibitor, or combinations thereof. In some embodiments, a MEK inhibitor is selected from the group consisting of cobimetinib, trametinib, binimetinib, selumetinib, pimasertinib, refametinib, GDC-0623, PD-0325901, and BI-847325, or a pharmaceutically acceptable salt thereof. In some embodiments, the MEK inhibitor is cobimetinib or cobimetinib hemifumarate. In some embodiments, the ERK inhibitor is ravoxertinib (GDC-0994) or ulixertinib (BVD-523), or a pharmaceutically acceptable salt thereof. In some embodiments, the ERK inhibitor is ravoxertinib or ravoxertinib besylate. In some embodiments, the BRAF inhibitor is selected from the group consisting of vemurafenib, dabrafenib, encorafenib (LGX818), GDC-0879, XL281, ARQ736, PLX3603, RAF265, and sorafenib, or a pharmaceutically acceptable salt thereof. In some embodiments, the BRAF inhibitor is vemurafenib. In some embodiments, the MAPK signaling inhibitor is a CRAF inhibitor. In some embodiments, the RAF inhibitor is a pan-RAF inhibitor. In some embodiments, the pan-RAF inhibitor is selected from the group consisting of LY-3009120, HM95573, LXH-254, MLN2480, BeiGene-283, RXDX-105, BAL3833, regorafenib, and sorafenib, or a pharmaceutically acceptable salt thereof.

In other embodiments of any one of the first, second, third, fourth, and fifth aspects, the method further comprises administering to the patient an additional therapeutic agent. In some embodiments, the additional therapeutic agent is an additional MAPK signaling inhibitor. In some embodiments, the MAPK signaling inhibitors are co-administered. In some embodiments, the MAPK signaling inhibitors are sequentially administered. In some embodiments, the method comprises administering cobimetinib and vemurafenib, or pharmaceutically acceptable salts thereof. In other embodiments, the additional therapeutic agent is an anti-cancer agent. In some embodiments, the anti-cancer agent and the one or more MAPK signaling inhibitors are co-administered. In some embodiments, the anti-cancer agent and the one or more MAPK signaling inhibitors are sequentially administered. In some embodiments, the anti-cancer agent is selected from the group consisting of a chemotherapeutic agent, a growth inhibitory agent, a cytotoxic agent, an agent used in radiation therapy, an anti-angiogenesis agent, an apoptotic agent, an anti-tubulin agent, and an immunotherapy agent. In some embodiments, the anti-cancer agent is a chemotherapeutic agent.

In a six aspect, the invention features a kit for identifying a patient who may benefit from treatment comprising one or more MAPK signaling inhibitors, the kit comprising polypeptides or polynucleotides capable of determining the expression level of the at least one gene (e.g., one, two, three, four, five, six, seven, eight, nine, or ten genes) selected from the group consisting of DUSP6, ETV4, SPRY2, SPRY4, PHLDA1, ETV5, DUSP4, CCND1, EPHA2, and EPHA4 and instructions for using the polypeptides or polynucleotides to identify a patient that may benefit from treatment comprising one or more MAPK signaling inhibitors.

In a seventh aspect, the invention features a composition comprising polypeptides or polynucleotides capable of determining the expression level of at least four genes (e.g., four, five, six, seven, eight, nine, or ten genes) selected from the group consisting of DUSP6, ETV4, SPRY2, SPRY4, PHLDA1, ETV5, DUSP4, CCND1, EPHA2, and EPHA4.

### BRIEF DESCRIPTION OF THE DRAWINGS

FIG. 1A is a graph showing how the short list and long list of genes associated with MEK inhibitor sensitivity were derived by the elastic-net model.

FIG. 1B is a graph showing cross-validation of the elastic-net model as assessed by correlating the predicted mean viabilities of cell lines used to create the model with experimentally derived mean viabilities to both trametinib and cobimetinib.

FIG. 1C is a graph showing a high correlation between the predicted mean viabilities to both trametinib and cobimetinib.

FIG. 1D is a series of graphs showing the correlation of the predicted mean viabilities to trametinib (right) and cobimetinib (left) with experimentally derived mean viabilities from 40 previously unscreened NSCLC cell lines, which were not used to derive the elastic-net model. R values for all data represent the Spearman correlation coefficient.

FIG. 2A is a diagram showing the genes present on the short-list associated with sensitivity to either trametinib, cobimetinib, or both drugs. The model groups each gene with other similarly correlated genes to form a gene feature set. The seven underlined gene feature sets (left column) contain genes whose expression is correlated with high MEK inhibitor sensitivity. The 14 italicized gene feature sets (right column) contain gene whose expression is inversely correlated with MEK inhibitor sensitivity.

FIG. 2B is a heatmap showing the clustering of genes according to expression in sensitive versus resistant cell lines. Short-list coefficients are shown along the y-axis, with higher coefficients indicating stronger predictive value. Expression data are normalized variance transformed RNA-Seq data with mean=0 and standard deviation=1.

FIG. 2C is a chart showing additional MAPK-specific genes present in the PHLDA1 gene feature set that are highly correlated with PHLDA1 gene expression derived from RNA-Seq data.

FIG. 3A is a series of graphs showing MAPK gene expression (top) and tumor volume changes (bottom) in C57B15 mice from an NSCLC GEM model (LSL-KrasG12D/+, P53FRT/FRT-Adeno-CRE) treated with vehicle (medium chained triglycerides (MCT)), cobimetinib (5 mg/kg), GDC-0994 (60 mg/kg), or a combination of both cobimetinib and GDC-0994, administered orally once a day for 14 days. Tumor volume changes at day 14 and RNA were collected six hours post-last dose following four days treatment. The RNA was analyzed by Nanostring to measure MAPK gene expression. Data show tumor volume as a percent change from baseline. MAPK gene expression data are shown as relative transcript abundance as a percent of the vehicle control.

FIG. 3B are graphs showing gene expression data (RNA-Seq) from ten MAPK-specific genes that were aggregated to create a MAPK activity score. The MAPK activity score correlated with sensitivity (mean viability) of >1000 cell lines to 95 drugs, including MAPK pathway inhibitors (RAF, MEK and ERK inhibitors), across multiple indications, including lung, breast (BRCA), CRC (colorectal), and melanoma (left). The inverse correlation of MAPK activity score to mean viability to cobimetinib is also shown in the right panel.

FIG. 3C is a series of graphs showing accuracy (top), receiver operating characteristics (ROC) curves (bottom left), and area under the curves (AUC) (bottom right) data for classifying cobimetinib sensitivity. The accuracy and false positive (FP)/false negative (FN) rate comparison of the elastic-net model, MAPK activity score, and KRAS mutation status are shown. The threshold for calling “sensitive” versus “resistant” was varied from 0-100% biomarker-positive cells over 5% intervals. ROC curves were generated by similarly varying the threshold for calling sensitive versus resistant cell lines and calculating FP and FN rates at each point for each predictor. As a negative control, an activity score computed from four non-MAPK genes was also included for comparison. The ROC curve data are summarized as AUC by subtracting the zero predictive value line from the data.

FIG. 4A is a heatmap showing correlation of gene expression data (RNA-Seq) from each individual MAPK-specific gene that makes up the score to sensitivity (mean viability) of >1000 cell lines to cobimetinib across multiple indications.

FIG. 4B is a heatmap showing the correlation of gene expression data (RNA-Seq) from each individual MAPK-specific gene that makes up the MAPK activity score with sensitivity (mean viability) of >1000 cell lines to MAPK pathway signaling inhibitors.

FIG. 5A is a graph showing MAPK activity scores computed for all tumor samples across different indications represented in The Cancer Genome Atlas (TCGA), classified by mutation status: BRAF mutant, RAS mutant, PI3K mutant compared to wild-type and normal tissue.

FIG. 5B is a graph showing MAPK activity scores computed for all tumor samples with different mutations represented in TCGA, classified by tissue type.

FIG. 5C is a series of graphs comparing clinical gene expression to cell line drug sensitivity to cobimetinib. The average MAPK activity score for each tissue type as measured in TCGA was correlated to the average mean viability for cell lines of the same tissue type for all samples (top left), BRAF-mutant samples (top right), RAS-mutant samples (bottom left), and wild-type samples (bottom right).

FIG. 6A is a graph showing Kaplan-Meier curves for progression-free survival (PFS) of MAPK-high and MAPK-low patients, classified as being above or below the median value of the MAPK activity score, respectively. Cox-proportional hazard regression models were then used to fit each treatment arm separately, using MAPK-high and MAPK-low as independent predictors of PFS to calculate the hazard ratio (HR) and associated p-values.

FIG. 6B is a graph showing Kaplan-Meier curves for progression-free survival (PFS) of MAPK-high and MAPK-low patients, further classified according to previously characterized baseline gene expression signatures: Cell Cycle (highly proliferative tumors with a low immune infiltrate) and Immune (higher immune infiltrate tumors with slower proliferation).

### DETAILED DESCRIPTION OF THE INVENTION

### I. Introduction

The present invention provides diagnostic methods, therapeutic methods, and compositions for the treatment of proliferative cell disorders (e.g., cancer (e.g., lung cancer, breast cancer, skin cancer, colorectal cancer, stomach cancer, lymphoid cancer, ovarian cancer, and cervical cancer)). The invention is based, at least in part, on the discovery that mitogen-activated protein kinase (MAPK) expression levels of particular MAPK-responsive genes (e.g., DUSP6, ETV4, SPRY2, SPRY4, PHLDA1, ETV5, DUSP4, CCND1, EPHA2, and EPHA4) can be used as biomarkers (e.g., predictive biomarkers) in methods of predicting sensitivity to treatment including MAPK signaling inhibitor(s); optimizing therapeutic efficacy for treatment including MAPK signaling inhibitor(s); selecting a therapy including MAPK signaling inhibitor(s) for a patient having a cancer; and treating a patient having a cancer with a therapy including MAPK signaling inhibitor(s). In some instances, a MAPK activity score based on the expression levels of one or more MAPK-responsive genes may be used to predict responsiveness to treatment including MAPK signaling inhibitor(s). The invention also provides methods of using the expression levels of the MAPK-responsive genes as prognostic biomarkers because patients with high MAPK activity scores can be expected to have a better outcome than patients with low MAPK activity scores.

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The term “MAPK signaling pathway” refers to the mitogen-activated protein kinase signaling pathway (e.g., the RAS/RAF/MEK/ERK signaling pathway) and encompasses a family of conserved serine/threonine protein kinases (e.g., the mitogen-activated protein kinases (MAPKs)). Abnormal regulation of the MAPK pathway contributes to uncontrolled proliferation, invasion, metastases, angiogenesis, and diminished apoptosis. The RAS family of GTPases includes KRAS, HRAS, and NRAS. The RAF family of serine/threonine protein kinases includes ARAF, BRAF, and CRAF (RAF1). Exemplary MAPKs include the extracellular signal-regulated kinase 1 and 2 (i.e., ERK1 and ERK2), the c-Jun N-terminal kinases 1-3 (i.e., JNK1, JNK2, and JNK3), the p38 isoforms (i.e., p38α, p38β, p38γ, and p38δ), and Erk5. Additional MAPKs include Nemo-like kinase (NLK), Erk3/4 (i.e., ERK3 and ERK4), and Erk7/8 (i.e., ERK7 and ERK8).

The term “MAPK signaling inhibitor,” “MAPK signaling antagonist,” “MAPK pathway inhibitor,” or “MAPK pathway signaling inhibitor” refers to a molecule that decreases, blocks, inhibits, abrogates, or interferes with signal transduction through the MAPK pathway (e.g., the RAS/RAF/MEK/ERK pathway). In some embodiments, a MAPK signaling inhibitor may inhibit the activity of one or more proteins involved in the activation of MAPK signaling. In some embodiments, a MAPK signaling inhibitor may activate the activity of one or more proteins involved in the inhibition of MAPK signaling. MAPK signaling inhibitors include, but are not limited to, MEK inhibitors (e.g., MEK1 inhibitors, MEK2 inhibitors, and inhibitors of both MEK1 and MEK2), RAF inhibitors (e.g., ARAF inhibitors, BRAF inhibitors, CRAF inhibitors, and pan-RAF inhibitors (i.e., RAF inhibitors that are inhibiting more than one member of the RAF family (i.e., two or all three of ARAF, BRAF, and CRAF)), and ERK inhibitors (e.g., ERK1 inhibitors and ERK2 inhibitors).

The term “BRAF inhibitor” or “BRAF antagonist” refers to molecule that decreases, blocks, inhibits, abrogates, or interferes with BRAF activation or function. In a particular embodiment, a BRAF inhibitor has a binding affinity (dissociation constant) to BRAF of about 1,000 nM or less. In another embodiment, a BRAF inhibitor has a binding affinity to BRAF of about 100 nM or less. In another embodiment, a BRAF inhibitor has a binding affinity to BRAF of about 50 nM or less. In another embodiment, a BRAF inhibitor has a binding affinity to BRAF of about 10 nM or less. In another embodiment, a BRAF inhibitor has a binding affinity to BRAF of about 1 nM or less. In a particular embodiment, a BRAF inhibitor inhibits BRAF signaling with an IC50 of 1,000 nM or less. In another embodiment, a BRAF inhibitor inhibits BRAF signaling with an IC50 of 500 nM or less. In another embodiment, a BRAF inhibitor inhibits BRAF signaling with an IC50 of 50 nM or less. In another embodiment, a BRAF inhibitor inhibits BRAF signaling with an IC50 of 10 nM or less. In another embodiment, a BRAF inhibitor inhibits BRAF signaling with an IC50 of 1 nM or less. Examples of BRAF inhibitors that may be used in accordance with the invention include, without limitation, vemurafenib (ZELBORAF®), dabrafenib, encorafenib (LGX818), GDC-0879, XL281, ARQ736, PLX3603, RAF265, and sorafenib, or a pharmaceutically acceptable salt thereof. BRAF inhibitors may inhibit only BRAF or may inhibit BRAF and one or more additional targets. Preferred BRAF inhibitors as described in PCT Application Publication Nos. WO 2005/062795, WO 2007/002325, WO 2007/002433, WO 2008/079903, and WO 2008/079906, which are each incorporated herein by reference in its entirety.

The term “ERK inhibitor” or “ERK antagonist” refers to molecule that decreases, blocks, inhibits, abrogates, or interferes with ERK (e.g., ERK1 and/or ERK2) activation or function. In a particular embodiment, an ERK inhibitor has a binding affinity (dissociation constant) to ERK of about 1,000 nM or less. In another embodiment, an ERK inhibitor has a binding affinity to ERK of about 100 nM or less. In another embodiment, an ERK inhibitor has a binding affinity to ERK of about 50 nM or less. In another embodiment, an ERK inhibitor has a binding affinity to ERK of about 10 nM or less. In another embodiment, an ERK inhibitor has a binding affinity to ERK of about 1 nM or less. In a particular embodiment, an ERK inhibitor inhibits ERK signaling with an IC50 of 1,000 nM or less. In another embodiment, an ERK inhibitor inhibits ERK signaling with an IC50 of 500 nM or less. In another embodiment, an ERK inhibitor inhibits ERK signaling with an IC50 of 50 nM or less. In another embodiment, an ERK inhibitor inhibits ERK signaling with an IC50 of 10 nM or less. In another embodiment, an ERK inhibitor inhibits ERK signaling with an IC50 of 1 nM or less. Examples of ERK inhibitors that may be used in accordance with the invention include, without limitation, ravoxertinib (GDC-0994) and ulixertinib (BVD-523), or a pharmaceutically acceptable salt (e.g., a besylate salt (e.g., a besylate salt of ravoxertinib)) thereof. ERK inhibitors may inhibit only ERK or may inhibit ERK and one or more additional targets. Preferred ERK inhibitors as described in PCT Application Publication Nos. WO 2013/130976, WO 2012/118850, WO 2013/020062, WO 2015/154674, WO 2015/085007, WO 2015/032840, WO 2014/036015, WO 2014/060395, WO 2015/103137, and WO 2015/103133, which are each incorporated herein by reference in its entirety.

The term “MEK inhibitor” or “MEK antagonist” refers to molecule that decreases, blocks, inhibits, abrogates, or interferes with MEK (e.g., MEK1 and/or MEK2) activation or function. In a particular embodiment, a MEK inhibitor has a binding affinity (dissociation constant) to MEK of about 1,000 nM or less. In another embodiment, a MEK inhibitor has a binding affinity to MEK of about 100 nM or less. In another embodiment, a MEK inhibitor has a binding affinity to MEK of about 50 nM or less. In another embodiment, a MEK inhibitor has a binding affinity to MEK of about 10 nM or less. In another embodiment, a MEK inhibitor has a binding affinity to MEK of about 1 nM or less. In a particular embodiment, a MEK inhibitor inhibits MEK signaling with an IC50 of 1,000 nM or less. In another embodiment, a MEK inhibitor inhibits MEK signaling with an IC50 of 500 nM or less. In another embodiment, a MEK inhibitor inhibits MEK signaling with an IC50 of 50 nM or less. In another embodiment, a MEK inhibitor inhibits MEK signaling with an IC500 of 10 nM or less. In another embodiment, a MEK inhibitor inhibits MEK signaling with an IC50 of 1 nM or less. Examples of MEK inhibitors that may be used in accordance with the invention include, without limitation, cobimetinib (e.g., cobimetinib hemifumarate; COTELLIC®), trametinib, binimetinib, selumetinib, pimasertinib, refametinib, GDC-0623, PD-0325901, and BI-847325, or a pharmaceutically acceptable salt thereof. MEK inhibitors may inhibit only MEK or may inhibit MEK and one or more additional targets. Preferred MEK inhibitors as described in PCT Application Publication Nos. WO 2007/044515, WO 2008/024725, WO 2008/024724, WO 2008/067481, WO 2008/157179, WO 2009/085983, WO 2009/085980, WO 2009/082687, WO 2010/003025, and WO 2010/003022, which are each incorporated herein by reference in its entirety.

The term “CRAF inhibitor” or “CRAF antagonist” refers to molecule that decreases, blocks, inhibits, abrogates, or interferes with CRAF activation or function. In a particular embodiment, a CRAF inhibitor has a binding affinity (dissociation constant) to CRAF of about 1,000 nM or less. In another embodiment, a CRAF inhibitor has a binding affinity to CRAF of about 100 nM or less. In another embodiment, a CRAF inhibitor has a binding affinity to CRAF of about 50 nM or less. In another embodiment, a CRAF inhibitor has a binding affinity to CRAF of about 10 nM or less. In another embodiment, a CRAF inhibitor has a binding affinity to CRAF of about 1 nM or less. In a particular embodiment, a CRAF inhibitor inhibits CRAF signaling with an IC50 of 1,000 nM or less. In another embodiment, a CRAF inhibitor inhibits CRAF signaling with an IC50 of 500 nM or less. In another embodiment, a CRAF inhibitor inhibits CRAF signaling with an IC50 of 50 nM or less. In another embodiment, a CRAF inhibitor inhibits CRAF signaling with an IC50 of 10 nM or less. In another embodiment, a CRAF inhibitor inhibits CRAF signaling with an IC50 of 1 nM or less. Examples of CRAF inhibitors that may be used in accordance with the invention include, without limitation, sorafenib, or a pharmaceutically acceptable salt thereof. CRAF inhibitors may inhibit only CRAF or may inhibit CRAF and one or more additional targets.

The term “pan-RAF inhibitor” or “pan-RAF antagonist” refers to a molecule that decreases, blocks, inhibits, abrogates, or interferes with the activation or function of two or more RAF family members (e.g., two or more of ARAF, BRAF, and CRAF). In one embodiment, the pan-RAF inhibitor inhibits all three RAF family members (i.e., ARAF, BRAF, and CRAF) to some extent. In a particular embodiment, a pan-RAF inhibitor has a binding affinity (dissociation constant) to one, two, or three of ARAF, BRAF, and/or CRAF of about 1,000 nM or less. In another embodiment, a pan-RAF inhibitor has a binding affinity to one, two, or three of ARAF, BRAF, and/or CRAF of about 100 nM or less. In another embodiment, a pan-RAF inhibitor has a binding affinity to one, two, or three of ARAF, BRAF, and/or CRAF of about 50 nM or less. In another embodiment, a pan-RAF inhibitor has a binding affinity to one, two, or three of ARAF, BRAF, and/or CRAF of about 10 nM or less. In another embodiment, a pan-RAF inhibitor has a binding affinity to one, two, or three of ARAF, BRAF, and/or CRAF of about 1 nM or less. In a particular embodiment, a pan-RAF inhibitor inhibits ARAF, BRAF, and/or CRAF signaling with an IC50 of 1,000 nM or less. In another embodiment, a pan-RAF inhibitor inhibits ARAF, BRAF, and/or CRAF signaling with an IC50 of 500 nM or less. In another embodiment, a pan-RAF inhibitor inhibits ARAF, BRAF, and/or CRAF signaling with an IC50 of 50 nM or less. In another embodiment, a pan-RAF inhibitor inhibits ARAF, BRAF, and/or CRAF signaling with an IC50 of 10 nM or less. In another embodiment, a pan-RAF inhibitor inhibits ARAF, BRAF, and/or CRAF signaling with an IC50 of 1 nM or less. Examples of pan-RAF inhibitors that may be used in accordance with the invention include, without limitation, LY-3009120, HM95573, LXH-254, MLN2480, BeiGene-283, RXDX-105, BAL3833, regorafenib, and sorafenib, or a pharmaceutically acceptable salt thereof. Pan-RAF inhibitors may inhibit ARAF, BRAF, and/or CRAF and one or more additional targets. Preferred pan-RAF inhibitors are described in PCT Application Publication Nos. WO2013/100632, WO2014/151616, and WO2015/075483, which are each incorporated herein by reference in its entirety.

The term “gene feature set” refers to a set of genes (e.g., DUSP6, ETV4, SPRY2, SPRY4, PHLDA1, ETV5, DUSP4, CCND1, EPHA2, and EPHA4) whose expression levels correlate directly with each other. As used herein, a gene feature set may be associated with predicted sensitivity to MAPK signaling inhibition.

The term “MAPK activity score” refers to a measurement of MAPK activity (e.g., an aggregate measurement of the expression levels of MAPK genes (e.g., an aggregate measurement of the expression level of DUSP6, ETV4, SPRY2, SPRY4, PHLDA1, ETV5, DUSP4, CCND1, EPHA2, and EPHA4)). As used herein, a MAPK activity score may be determined according to the algorithm:

$$\frac{Σ {z}_{i}}{\sqrt{n}} ,$$

where zᵢ is the z-score of each gene, normalized across all samples, or to a set of housekeeping genes, and n is the number of genes comprising the set, and can be used to identify a patient having an increased benefit to treatment including a MAPK signaling inhibitor. A “z-score” is a statistical measurement of the expression level of an individual biomarker (e.g., an individual gene) to the mean value of the expression level of a biomarker across a data set (e.g., a population or group of multiple samples). In some instances, a z-score of zero means that a biomarker expression level is the same as the mean. In some instances, a z-score can also be positive or negative, indicating that a biomarker expression level is above or below the population mean, respectively. In some instances, the expression level of a biomarker is a set of genes that is expressed at a stable level. In some instances, the z-score of each gene is measured in reads per kilobase per million (RPKM) (e.g., by RNA-Seq). In some instances, the set of housekeeping genes that may be used are MLH1, SMARCA4, U2AF1, and CLTC. In some instances, the MAPK activity score is greater than the median MAPK activity score determined for a set of samples (e.g., tissue samples from one or more patients having a cancer (e.g., tumor tissue samples)). In some instances, a MAPK activity score greater than the median MAPK activity score identifies a patient with increased likelihood of benefiting from a treatment including a MAPK signaling inhibitor. In some instances, a MAPK activity score less than the median MAPK activity core identifies a patient with a reduced likelihood of benefiting from a treatment including a MAPK signaling inhibitor.

The term “PHLDA1” refers to Pleckstrin homology-like domain family A member 1 and encompasses homologues, mutations, and isoforms thereof. PHLDA1 is also referred to in the art as PHRIP or TDAG51. The term encompasses full-length, unprocessed PHLDA1, as well as any form of PHLDA1 that results from processing in the cell. The term encompasses naturally occurring variants of PHLDA1 (e.g., splice variants or allelic variants). The term encompasses, for example, the PHLDA1 gene, the mRNA sequence of human PHLDA1 (e.g., SEQ ID NO: 1; GenBank Accession No. NM_007350.3), and the amino acid sequence of human PHLDA1 (e.g., SEQ ID NO: 2; UniProtKB Accession No. Q8WV24) as well as PHLDA1 DNA, mRNA, and amino acid sequences from any other vertebrate source, including mammals such as primates and rodents (e.g., mice and rats).

The term “SPRY2” refers to Protein sprouty homolog 2 and encompasses homologues, mutations, and isoforms thereof. The term encompasses full-length, unprocessed SPRY2, as well as any form of SPRY2 that results from processing in the cell. The term encompasses naturally occurring variants of SPRY2 (e.g., splice variants or allelic variants). The term encompasses, for example, the SPRY2 gene, the mRNA sequence of human SPRY2 (e.g., SEQ ID NO: 3; GenBank Accession No. NM_001318536.1), and the amino acid sequence of human SPRY2 (e.g., SEQ ID NO: 4; UniProtKB Accession No. O43597) as well as SPRY2 DNA, mRNA, and amino acid sequences from any other vertebrate source, including mammals such as primates and rodents (e.g., mice and rats).

The term “SPRY4” refers to Protein sprouty homolog 4 and encompasses homologues, mutations, and isoforms thereof. The term encompasses full-length, unprocessed SPRY4, as well as any form of SPRY4 that results from processing in the cell. The term encompasses naturally occurring variants of SPRY4 (e.g., splice variants or allelic variants). The term encompasses, for example, the SPRY4 gene, the mRNA sequence of human SPRY4 (e.g., SEQ ID NO: 5; GenBank Accession No. NM_001127496.1), and the amino acid sequence of human SPRY4 (e.g., SEQ ID NO: 6; UniProtKB Accession No. Q9C004) as well as SPRY4 DNA, mRNA, and amino acid sequences from any other vertebrate source, including mammals such as primates and rodents (e.g., mice and rats).

The term “DUSP4” refers to Dual specificity protein phosphatase 4 (e.g., Mitogen-activated protein kinase phosphatase 2 (e.g., MAP kinase phosphatase 2)) and encompasses homologues, mutations, and isoforms thereof. DUSP4 is also referred to in the art as MKP2 or VH2. The term encompasses full-length, unprocessed DUSP4, as well as any form of DUSP4 that results from processing in the cell. The term encompasses naturally occurring variants of DUSP4 (e.g., splice variants or allelic variants). The term encompasses, for example, the DUSP4 gene, the mRNA sequence of human DUSP4 (e.g., SEQ ID NO: 7; GenBank Accession No. NM_001394.6), and the amino acid sequence of human DUSP4 (e.g., SEQ ID NO: 8; UniProtKB Accession No. Q13115) as well as DUSP4 DNA, mRNA, and amino acid sequences from any other vertebrate source, including mammals such as primates and rodents (e.g., mice and rats).

The term “DUSP6” refers to Dual specificity protein phosphatase 6 (e.g., Mitogen-activated protein kinase phosphatase 3 (e.g., MAP kinase phosphatase 3) and encompasses homologues, mutations, and isoforms thereof. DUSP6 is also referred to in the art as MKP3 or PYST1. The term encompasses full-length, unprocessed DUSP6, as well as any form of DUSP6 that results from processing in the cell. The term encompasses naturally occurring variants of DUSP6 (e.g., splice variants or allelic variants). The term encompasses, for example, the DUSP6 gene, the mRNA sequence of human DUSP6 (e.g., SEQ ID NO: 9; GenBank Accession No. NM_022652.3), and the amino acid sequence of human DUSP6 (e.g., SEQ ID NO: 10; UniProtKB Accession No. Q16828) as well as DUSP6 DNA, mRNA, and amino acid sequences from any other vertebrate source, including mammals such as primates and rodents (e.g., mice and rats).

The term “CCND1” refers to GVS-specific cyclin-D1 and encompasses homologues, mutations, and isoforms thereof. CCND1 is also referred to in the art as BCL1 or PRAD1. The term encompasses full-length, unprocessed CCND1, as well as any form of CCND1 that results from processing in the cell. The term encompasses naturally occurring variants of CCND1 (e.g., splice variants or allelic variants). The term encompasses, for example, the CCND1 gene, the mRNA sequence of human CCND1 (e.g., SEQ ID NO: 11; GenBank Accession No. NM_053056.2), and the amino acid sequence of human CCND1 (e.g., SEQ ID NO: 12; UniProtKB Accession No. P24385) as well as CCND1 DNA, mRNA, and amino acid sequences from any other vertebrate source, including mammals such as primates and rodents (e.g., mice and rats).

The term “EPHA2” refers to Ephrin type-A receptor 2 and encompasses homologues, mutations, and isoforms thereof. EPHA2 is also referred to in the art as ECK. The term encompasses full-length, unprocessed EPHA2, as well as any form of EPHA2 that results from processing in the cell. The term encompasses naturally occurring variants of EPHA2 (e.g., splice variants or allelic variants). The term encompasses, for example, the EPHA2 gene, the mRNA sequence of human EPHA2 (e.g., SEQ ID NO: 13; GenBank Accession No. NM_004431.3), and the amino acid sequence of human EPHA2 (e.g., SEQ ID NO: 14; UniProtKB Accession No. P29317) as well as EPHA2 DNA, mRNA, and amino acid sequences from any other vertebrate source, including mammals such as primates and rodents (e.g., mice and rats).

The term “EPHA4” refers to Ephrin type-A receptor 4 and encompasses homologues, mutations, and isoforms thereof. EPHA4 is also referred to in the art as HEK8, SEK, or TYRO1. The term encompasses full-length, unprocessed EPHA4, as well as any form of EPHA4 that results from processing in the cell. The term encompasses naturally occurring variants of EPHA4 (e.g., splice variants or allelic variants). The term encompasses, for example, the EPHA4 gene, the mRNA sequence of human EPHA4 (e.g., SEQ ID NO: 15; GenBank Accession No. NM_001304536.1), and the amino acid sequence of human EPHA4 (e.g., SEQ ID NO: 16; UniProtKB Accession No. P54764) as well as EPHA4 DNA, mRNA, and amino acid sequences from any other vertebrate source, including mammals such as primates and rodents (e.g., mice and rats).

The term “ETV4” refers to ETS translocation variant 4 and encompasses homologues, mutations, and isoforms thereof. ETV4 is also referred to in the art as E1AF or PEA3. The term encompasses full-length, unprocessed ETV4, as well as any form of ETV4 that results from processing in the cell. The term encompasses naturally occurring variants of ETV4 (e.g., splice variants or allelic variants). The term encompasses, for example, the ETV4 gene, the mRNA sequence of human ETV4 (e.g., SEQ ID NO: 17; GenBank Accession No. NM_001261437.1), and the amino acid sequence of human ETV4 (e.g., SEQ ID NO: 18; UniProtKB Accession No. P43268) as well as ETV4 DNA, mRNA, and amino acid sequences from any other vertebrate source, including mammals such as primates and rodents (e.g., mice and rats).

The term “ETV5” refers to ETS translocation variant 5 and encompasses homologues, mutations, and isoforms thereof. ETV5 is also referred to in the art as ERM. The term encompasses full-length, unprocessed ETV5, as well as any form of ETV5 that results from processing in the cell. The term encompasses naturally occurring variants of ETV5 (e.g., splice variants or allelic variants). The term encompasses, for example, the ETV5 gene, the mRNA sequence of human ETV5 (e.g., SEQ ID NO: 19; GenBank Accession No. NM_004454.2), and the amino acid sequence of human ETV5 (e.g., SEQ ID NO: 20; UniProtKB Accession No. P41161) as well as ETV5 DNA, mRNA, and amino acid sequences from any other vertebrate source, including mammals such as primates and rodents (e.g., mice and rats).

A “patient” or “subject” herein refers to an animal (including, e.g., a mammal, such as a dog, a cat, a horse, a rabbit, a zoo animal, a cow, a pig, a sheep, a non-human primate, and a human), eligible for treatment who is experiencing, has experienced, has risk of developing, or has a family history of one or more signs, symptoms, or other indicators of a cell proliferative disease or disorder, such as a cancer. Intended to be included as a patient is any patient involved in clinical research trials not showing any clinical sign of disease, involved in epidemiological studies, or once used as controls. The patient may have been previously treated with a MAPK signaling inhibitor, another drug, or not previously treated. The patient may be naive to an additional drug(s) being used when the treatment is started, i.e., the patient may not have been previously treated with, for example, a therapy other than one including a MAPK signaling inhibitor (e.g., a MEK inhibitor, a BRAF inhibitor, an ERK inhibitor, a CRAF inhibitor, or a RAF inhibitor) at “baseline” (i.e., at a set point in time before the administration of a first dose of a MAPK pathway inhibitor in the treatment method herein, such as the day of screening the subject before treatment is commenced). Such a “naive” patient or subject is generally considered a candidate for treatment with such additional drug(s).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Polynucleotide” or “nucleic acid,” as used interchangeably herein, refers to polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single-stranded polynucleotide that is capable of hybridizing to a nucleic acid and allowing polymerization of a complementary nucleic acid, generally by providing a free 3′-OH group.

The term “small molecule” refers to any molecule with a molecular weight of about 2000 daltons or less, preferably of about 500 daltons or less.

The term “detection” includes any means of detecting, including direct and indirect detection.

The term “biomarker” as used herein refers to an indicator molecule or set of molecules (e.g., predictive, diagnostic, and/or prognostic indicator), which can be detected in a sample and includes, for example, DUSP6, ETV4, SPRY2, SPRY4, PHLDA1, ETV5, DUSP4, CCND1, EPHA2, and EPHA4. The biomarker may be a predictive biomarker and serve as an indicator of the likelihood of sensitivity or benefit of a patient having a particular disease or disorder (e.g., a proliferative cell disorder (e.g., cancer)) to treatment with a MAPK signaling inhibitor. Biomarkers include, but are not limited to, polynucleotides (e.g., DNA and/or RNA (e.g., mRNA)), polynucleotide copy number alterations (e.g., DNA copy numbers), polypeptides, polypeptide and polynucleotide modifications (e.g., post-translational modifications), carbohydrates, and/or glycolipid-based molecular markers. In some embodiments, a biomarker is a gene.

The “amount” or “level” of a biomarker, as used herein, is a detectable level in a biological sample. These can be measured by methods known to one skilled in the art and also disclosed herein.

The term “level of expression” or “expression level” generally refers to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients.

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients. In some embodiments, reduced expression is little or no expression.

The term “housekeeping gene” refers herein to a gene or group of genes that encode proteins whose activities are essential for the maintenance of cell function and which are typically similarly present in all cell types. In some embodiments, the housekeeping gene can be MLH1, SMARCA4, U2AF1, and/or CLTC.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “sample,” as used herein, refers to a composition that is obtained or derived from a subject (e.g., individual of interest) that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples (e.g., tumor tissue samples), primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference level, reference sample, reference cell, reference tissue, control sample, control cell, or control tissue is obtained from a healthy and/or non-diseased part of the body (e.g., tissue or cells) of the same subject or individual. For example, the reference level, reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference level, reference sample, reference cell, reference tissue, control sample, control cell, or control tissue is obtained from a healthy and/or non-diseased part of the body (e.g., tissues or cells) of an individual who is not the subject or individual. In even another embodiment, a reference level, reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or having a, a disease or disorder, such as cancer. In one embodiment, such benefit includes any one or more of: extending survival (including overall survival and/or progression-free survival); resulting in an objective response (including a complete response or a partial response); or improving signs or symptoms of cancer. In one embodiment, at least one biomarker (e.g., the expression of DUSP6, ETV4, SPRY2, SPRY4, PHLDA1, ETV5, DUSP4, CCND1, EPHA2, and/or EPHA4) is used to identify a patient who is predicted to have an increased likelihood of being responsive to treatment with a medicament (e.g., treatment comprising a MAPK signaling inhibitor), relative to a patient who does not express the at least one biomarker. In one embodiment, the at least one biomarker (e.g., the expression level of DUSP6, ETV4, SPRY2, SPRY4, PHLDA1, ETV5, DUSP4, CCND1, EPHA2, and/or EPHA4) is used to identify the patient who is predicted to have an increase likelihood of being responsive to treatment with a medicament (e.g., MAPK signaling inhibitor), relative to a patient who does not express the at least one biomarker at the same level.

An “objective response” refers to a measurable response, including complete response (CR) or partial response (PR). In some embodiments, the “objective response rate (ORR)” refers to the sum of complete response (CR) rate and partial response (PR) rate.

By “complete response” or “CR” is intended the disappearance of all signs of a proliferative cell disorder such as cancer (e.g., disappearance of all target lesions) in response to treatment. This does not always mean the disease (e.g., cancer) has been cured.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration at least the same as the treatment duration, at least 1.5×, 2.0×, 2.5×, or 3.0× length of the treatment duration, or longer.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

The term “survival” refers to the patient remaining alive, and includes overall survival as well as progression-free survival

As used herein, “progression-free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” or “OS” refers to the percentage of individuals in a group who are likely to be alive after a particular duration of time.

By “extending survival” is meant increasing overall or progression-free survival in a treated patient relative to an untreated patient (i.e. relative to a patient not treated with the medicament), or relative to a patient who does not express a biomarker at the designated level, and/or relative to a patient treated with an anti-tumor agent.

A “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

A “disorder” is any condition that would benefit from treatment including, but not limited to, chronic and acute disorders or diseases including those pathological conditions which predispose the mammal to the disorder in question.

The terms “cancer” and “cancerous” refer to or describe the physiological condition in mammals that is typically characterized by unregulated cell growth. Included in this definition are benign and malignant cancers. Examples of cancer include, but are not limited to, carcinoma; lymphoma; blastoma (including medulloblastoma and retinoblastoma); sarcoma (including liposarcoma and synovial cell sarcoma); neuroendocrine tumors (including carcinoid tumors, gastrinoma, and islet cell cancer); mesothelioma; schwannoma (including acoustic neuroma); meningioma; adenocarcinoma; melanoma; and leukemia or lymphoid malignancies. More particular examples of such cancers include bladder cancer (e.g., urothelial bladder cancer (e.g., transitional cell or urothelial carcinoma, non-muscle invasive bladder cancer, muscle-invasive bladder cancer, and metastatic bladder cancer) and non-urothelial bladder cancer); squamous cell cancer (e.g., epithelial squamous cell cancer); lung cancer, including small-cell lung cancer (SCLC), non-small cell lung cancer (NSCLC), adenocarcinoma of the lung, and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; hepatoma; breast cancer (including metastatic breast cancer); colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; Merkel cell cancer; mycoses fungoids; testicular cancer; esophageal cancer; tumors of the biliary tract; head and neck cancer; and hematological malignancies. In some embodiments, the cancer is triple-negative metastatic breast cancer, including any histologically confirmed triple-negative (ER-, PR-, HER2-) adenocarcinoma of the breast with locally recurrent or metastatic disease (where the locally recurrent disease is not amenable to resection with curative intent). In some embodiments, the cancer is skin cancer, including melanoma with locally recurrent or metastatic disease (where the locally recurrent disease is not amenable to resection with curative intent). Any cancer can be at early stage or at late stage. By “early stage cancer” or “early stage tumor” is meant a cancer that is not invasive or metastatic or is classified as a Stage 0, 1, or 2 cancer.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

The term “pharmaceutically acceptable salt” denotes salts which are not biologically or otherwise undesirable. Pharmaceutically acceptable salts include both acid and base addition salts. The phrase “pharmaceutically acceptable” indicates that the substance or composition must be compatible chemically and/or toxicologically, with the other ingredients comprising a formulation, and/or the mammal being treated therewith.

The term “pharmaceutically acceptable acid addition salt” denotes those pharmaceutically acceptable salts formed with inorganic acids, such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines, including naturally occurring substituted amines, cyclic amines, and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, MAPK signaling inhibitors (e.g., MEK inhibitors, BRAF inhibitors, ERK inhibitors, CRAF inhibitors, and/or RAF inhibitors) are used to delay development of a disease or to slow the progression of a disease.

The term “anti-cancer therapy” refers to a therapy useful in treating cancer. Examples of anti-cancer therapeutic agents include, but are limited to, cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-β, BlyS, APRIL, BCMA receptor(s), TRAIL/Apo2, other bioactive and organic chemical agents, and the like. Combinations thereof are also included in the invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ⋅1I (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycin, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; gemcitabine (GEMZAR®); 6-thioguanine; mercaptopurine; methotrexate; platinum or platinum-based chemotherapy agents and platinum analogs, such as cisplatin, carboplatin, oxaliplatin (ELOXATIN™), satraplatin, picoplatin, nedaplatin, triplatin, and lipoplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts or acids of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

“Chemotherapeutic agents” also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts or acids of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant exclusively human-sequence, full-length IgG1 A antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see W098/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3, and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP 659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO 98/14451, WO 98/50038, WO 99/09016, and WO 99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitors such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell co-stimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); miscellaneous investigational agents such as thioplatin, PS-341, phenylbutyrate, ET-18-OCH3, and farnesyl transferase inhibitors (L-739749, L-744832); polyphenols such as quercetin, resveratrol, piceatannol, epigallocatechine gallate, theaflavins, flavanols, procyanidins, betulinic acid; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts or acids of any of the above; as well as combinations of two or more of the above.

The term “prodrug” as used herein refers to a precursor form of a pharmaceutically active substance that is less cytotoxic to tumor cells compared to the parent drug and is capable of being enzymatically activated or converted into the more active parent form. See, for example, Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth and/or proliferation of a cell (e.g., a cell whose growth is dependent on MAPK pathway signaling) either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-aphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in “The Molecular Basis of Cancer,” Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, “administering” is meant a method of giving a dosage of a compound (e.g., an inhibitor or antagonist) or a pharmaceutical composition (e.g., a pharmaceutical composition including an inhibitor or antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “co-administered” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the level of activity and/or function of a protein in the MAPK pathway (e.g., the level of signal transduction through the MAPK pathway). Additionally, Reduce or inhibit can refer, for example, to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., DUSP6, ETV4, SPRY2, SPRY4, PHLDA1, ETV5, DUSP4, CCND1, EPHA2, and EPHA4) described herein. In certain embodiments, the manufacture or kit is promoted, distributed, or sold as a unit for performing the methods described herein.

The phrase “based on” when used herein means that the information about one or more biomarkers is used to inform a diagnostic decision, a treatment decision, information provided on a package insert, or marketing/promotional guidance, etc.

### III. Methods

A. Diagnostic Methods Based on the Expression Level of MAPK Signaling Biomarkers

The present invention provides methods for identifying and/or monitoring patients having cancer (e.g., lung cancer, breast cancer, skin cancer, colorectal cancer, stomach cancer, lymphoid cancer, ovarian cancer, and cervical cancer) who may benefit from treatment including one or more mitogen-activated protein kinase (MAPK) signaling inhibitors. The methods include detecting expression of one or more biomarkers in a sample (e.g., a tissue sample (e.g., a tumor tissue sample)) from a patient, wherein the expression of one or more such biomarkers is indicative of whether the patient is sensitive or responsive to MAPK signaling inhibitors, such as MEK inhibitors, BRAF inhibitors, ERK inhibitors, and CRAF inhibitors. Also provided are methods for optimizing therapeutic efficacy for treatment of a patient having a cancer, wherein the treatment includes one or more MAPK signaling inhibitors. Further provided herein are methods for predicting responsiveness of a patient having a cancer to treatment including one or more MAPK signaling inhibitors. Also, provided herein are methods for selecting a therapy for a patient having a cancer. Any of the methods may further be based on the determination of a MAPK activity score, and/or a baseline gene expression signature. Any of the methods may further include administering to the patient a therapeutically effective amount of a MAPK signaling inhibitor to the patient. In addition, any of the methods may further include administering an effective amount of an additional therapeutic agent (e.g., a second) to the patient.

The invention provides methods for identifying a patient having a cancer who may benefit from treatment including one or more MAPK signaling inhibitors, predicting responsiveness of a patient having a cancer to treatment including one or more MAPK signaling inhibitors, and selecting a therapy for a patient having a cancer, based on determining an expression level of at least one (e.g., one, two, three, four, five, six, seven, eight, nine, or ten) gene selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment including one or more MAPK signaling inhibitors. More particularly, any of the preceding methods may be based on determining the expression level of at least one of the biomarkers provided herein, for example, determining the expression level of at least one (e.g., at least two, at least three, at least four, at least five, at least six, at least seven, at least eight, at least nine, or ten) of DUSP6, ETV4, SPRY2, SPRY4, PHLDA1, ETV5, DUSP4, CCND1, EPHA2, and EPHA4 in a sample from a patient useful for monitoring whether the patient is responsive or sensitive to MAPK signaling inhibition. For any of the methods described herein, one could, for example, determine the expression levels of any combination of 2, 3, 4, 5, 6, 7, 8, 9, or 10 genes selected from the biomarkers (e.g., genes) selected from DUSP6, ETV4, SPRY2, SPRY4, PHLDA1, ETV5, DUSP4, CCND1, EPHA2, and EPHA4. Alternatively, for any of the methods described herein, the expression level of all ten biomarkers (e.g., DUSP6, ETV4, SPRY2, SPRY4, PHLDA1, ETV5, DUSP4, CCND1, EPHA2, and EPHA4) can be determined.

The disclosed methods and assays provide for convenient, efficient, and potentially cost-effective means to obtain data and information useful in assessing appropriate or effective therapies for treating patients. For example, a patient can provide a tissue sample (e.g., a tumor biopsy or a blood sample) before and/or after treatment with a MAPK signaling inhibitor and the sample can be examined by way of various in vitro assays to determine whether the patient's cells are sensitive to a MAPK signaling inhibitors, such as MEK inhibitors, BRAF inhibitors, ERK inhibitors, and CRAF inhibitors.

The invention also provides methods for monitoring the sensitivity or responsiveness of a patient to a MAPK signaling inhibitor. The methods may be conducted in a variety of assay formats, including assays detecting genetic or protein expression levels and biochemical assays detecting appropriate activity. Determination of expression or the presence of such biomarkers in patient samples is predictive of whether a patient is sensitive to the biological effects of a MAPK signaling inhibitor. A difference or change (i.e., an increase) in the expression of at least one (e.g., one, two, three, four, five, six, seven, eight, nine, or ten) of the biomarkers of the invention (e.g., DUSP6, ETV4, SPRY2, SPRY4, PHLDA1, ETV5, DUSP4, CCND1, EPHA2, and EPHA4) in a sample from a patient having a cancer relative to a reference level (e.g., the median expression level of the biomarker in a sample from a group/population of patients being tested for responsiveness to a MAPK signaling inhibitor or the median expression level of the biomarker in a sample from a group/population of patients having a particular cancer) correlates with treatment efficacy of such a patient with a MAPK signaling inhibitor.

In one aspect, this invention provides a method of determining whether a patient having a cancer will respond to treatment with a MAPK signaling inhibitor including determining the expression level of at least one (e.g., one, two, three, four, five, six, seven, eight, nine, or ten) of the biomarkers selected from DUSP6, ETV4, SPRY2, SPRY4, PHLDA1, ETV5, DUSP4, CCND1, EPHA2, and EPHA4 in a sample from the patient obtained (i) before any MAPK signaling inhibitor has been administered to the patient, (ii) after any MAPK signaling inhibitor has been administered to the patient, or (iii) before and after such treatment. A change (e.g., increase) in the expression of the at least one or more biomarkers relative to a reference level indicates that the patient will likely respond to treatment with a MAPK signaling inhibitor. The patient may be informed that they have an increased likelihood of responding to treatment with a MAPK signaling inhibitor and/or provided a recommendation that anti-cancer therapy include a MAPK signaling inhibitor.

In another aspect, the invention provides a method of optimizing therapeutic efficacy of an anti-cancer therapy for a patient, including detecting, as a biomarker, expression of at least one (e.g., one, two, three, four, five, six, seven, eight, nine, or ten) of the genes selected from DUSP6, ETV4, SPRY2, SPRY4, PHLDA1, ETV5, DUSP4, CCND1, EPHA2, and EPHA4 in a sample from the patient obtained (i) before any MAPK signaling inhibitor has been administered to the patient, (ii) after any MAPK signaling inhibitor has been administered to the patient, or (iii) before and after such treatment. A change (e.g., increase) in the expression of the at least one of the biomarkers relative to a reference level indicates that the patient will likely respond to treatment with a MAPK signaling inhibitor. The patient may be informed that they have an increased likelihood of responding to treatment with a MAPK signaling inhibitor and/or provided a recommendation that anti-cancer therapy include a MAPK signaling inhibitor.

In another aspect, the invention provides a method for selecting a therapy for a patient having a cancer, including detecting, as a biomarker, the expression of at least one (e.g., one, two, three, four, five, six, seven, eight, nine, or ten) of the genes selected from DUSP6, ETV4, SPRY2, SPRY4, PHLDA1, ETV5, DUSP4, CCND1, EPHA2, and EPHA4 in a sample from the patient obtained (i) before any MAPK signaling inhibitor has been administered to the patient, (ii) after any MAPK signaling inhibitor has been administered to the patient, or (iii) before and after such treatment. A change (e.g., increase) in the expression of the at least one of the biomarkers relative to a reference level indicates that the patient will likely respond to treatment with a MAPK signaling inhibitor. The patient may be informed that they have an increased likelihood of responding to treatment with a MAPK signaling inhibitor and/or provided a recommendation that anti-cancer therapy include a MAPK signaling inhibitor.

In another embodiment, the present invention provides a method of monitoring the sensitivity or responsiveness of a patient to a MAPK signaling inhibitor. This method including assessing expression of at least one of the biomarkers selected from DUSP6, ETV4, SPRY2, SPRY4, PHLDA1, ETV5, DUSP4, CCND1, EPHA2, and EPHA4 in a patient sample and predicting the sensitivity or responsiveness of the patient to the MAPK signaling inhibitor, wherein a change (e.g., increase) in the expression of at least one biomarkers selected from DUSP6, ETV4, SPRY2, SPRY4, PHLDA1, ETV5, DUSP4, CCND1, EPHA2, and EPHA4 correlates with sensitivity or responsiveness of the patient to effective treatment with the MAPK signaling inhibitor. According to one embodiment of this method, a biological sample is obtained from the patient before administration of any MAPK signaling inhibitor and subjected to an assay to evaluate the level of expression products of at least one biomarker in the sample. If expression of at least one of the genes selected from DUSP6, ETV4, SPRY2, SPRY4, PHLDA1, ETV5, DUSP4, CCND1, EPHA2, and EPHA4 changed (i.e., increased) relative to a reference level, the patient is determined to be sensitive or responsive to treatment with a MAPK signaling inhibitor. The patient may be informed that they have an increased likelihood of being sensitive or responsive to treatment with a MAPK signaling inhibitor and/or provided a recommendation that anti-cancer therapy include a MAPK signaling inhibitor. In another embodiment of this method, a biological sample is obtained from the patient before and after administration of a MAPK signaling inhibitor, as described herein.

In any of the preceding methods, the expression level of at least one of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least one gene. In some instances, the expression level of at least two of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least two genes. In some instances, the expression level of at least three of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least three genes. In some instances, the expression level of at least four of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least four genes. In some instances, the expression level of at least five of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least five genes. In some instances, the expression level of at least six of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least six genes. In some instances, the expression level of at least seven of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least seven genes. In some instances, the expression level of at least eight of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least eight genes. In some instances, the expression level of at least nine of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least nine genes. In some instances, the expression level of all ten of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the ten genes.

In any of the preceding methods, an increased level of expression of PHLDA1, EPHA2, CCND1, SPRY2, SPRY4, ETV4, DUSP4, and/or DUSP6 relative to a reference level identifies a patient having a lung cancer as having an increased likelihood of benefit from treatment with a MAPK signaling inhibitor. In some instances, an increased level of expression of PHLDA1, SPRY2, ETV4, EPHA2, ETV5, and/or SPRY4 relative to a reference level identifies a patient having a breast cancer as having an increased likelihood of benefit from treatment with a MAPK signaling inhibitor. In some instances, an increased level of expression of DUSP4, SPRY4, and/or ETV4 relative to a reference level identifies a patient having a skin cancer as having an increased likelihood of benefit from treatment with a MAPK signaling inhibitor. In some instances, an increased level of expression of PHLDA1, DUSP6, SPRY4, and/or SPRY2 relative to a reference level identifies a patient having a colorectal cancer as having an increased likelihood of benefit from treatment with a MAPK signaling inhibitor. In some instances, an increased level of expression of DUSP4 relative to a reference level identifies a patient having a stomach cancer as having an increased likelihood of benefit from treatment with a MAPK signaling inhibitor. In some instances, an increased level of expression of DUSP6, ETV5, SPRY2, SPRY4, and/or ETV4 relative to a reference level identifies a patient having a lymphoid cancer as having an increased likelihood of benefit from treatment with a MAPK signaling inhibitor. In some instances, an increased level of expression of SPRY2 and/or DUSP6 relative to a reference level identifies a patient having an ovarian cancer as having an increased likelihood of benefit from treatment with a MAPK signaling inhibitor. In some instances, an increased level of expression of DUSP6 relative to a reference level identifies a patient having a cervical cancer as having an increased likelihood of benefit from treatment with a MAPK signaling inhibitor.

The presence and/or expression level (amount) of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, enzyme-linked immunosorbent assay (ELISA), enzyme-linked immunofiltration assay (ELIFA), fluorescence activated cell sorting (“FACS”), MassARRAY, proteomics, quantitative blood based assays (e.g., serum ELISA), biochemical enzymatic activity assays, in situ hybridization, fluorescence in situ hybridization (FISH), Southern analysis, Northern analysis, whole genome sequencing, polymerase chain reaction (PCR) (including quantitative real time PCR (qRT-PCR) and other amplification type detection methods, such as, for example, branched DNA, SISBA, TMA and the like), RNA-Seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In any of the preceding methods, the presence and/or expression level (amount) of a biomarker (e.g., DUSP6, ETV4, SPRY2, SPRY4, PHLDA1, ETV5, DUSP4, CCND1, EPHA2, and EPHA4) may be a nucleic acid expression level. In some instances, the nucleic acid expression level is determined using qPCR, rtPCR, RNA-Seq, multiplex qPCR or RT-qPCR, microarray analysis, SAGE, MassARRAY technique, or in situ hybridization (e.g., FISH). In some instances, the expression level of a biomarker (e.g., DUSP6, ETV4, SPRY2, SPRY4, PHLDA1, ETV5, DUSP4, CCND1, EPHA2, and EPHA4) is determined in tumor cells, tumor infiltrating immune cells, stromal cells, or combinations thereof.

In a particular instance, the expression level of a biomarker (e.g., DUSP6, ETV4, SPRY2, SPRY4, PHLDA1, ETV5, DUSP4, CCND1, EPHA2, and EPHA4) is an mRNA expression level. Methods for the evaluation of mRNAs in cells are well known and include, for example, RNA-Seq (e.g., whole transcriptome shotgun sequencing) using next generation sequencing techniques, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols that examine or detect mRNAs, such as target mRNAs, in a tissue or cell sample by microarray technologies. Using nucleic acid microarrays test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment including a MAPK signaling inhibitor may be arrayed on a solid support. Hybridization of a labeled probe with a particular array member indicates that the sample from which the probe was derived expresses that gene.

In any of the preceding methods, the presence and/or expression level (amount) of a biomarker (e.g., DUSP6, ETV4, SPRY2, SPRY4, PHLDA1, ETV5, DUSP4, CCND1, EPHA2, and EPHA4) is measured by determining protein expression levels of the biomarker. In certain instances, the method comprises contacting the biological sample with antibodies that specifically bind to a biomarker described herein under conditions permissive for binding of the biomarker, and detecting whether a complex is formed between the antibodies and biomarker. Such method may be an in vitro or in vivo method. Any method of measuring protein expression levels known in the art may be used. For example, in some instances, a protein expression level of a biomarker (e.g., DUSP6, ETV4, SPRY2, SPRY4, PHLDA1, ETV5, DUSP4, CCND1, EPHA2, and EPHA4) is determined using a method selected from the group consisting of flow cytometry (e.g., fluorescence-activated cell sorting (FACS™)), Western blot, ELISA, ELIFA, immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some instances, the protein expression level of the biomarker (e.g., DUSP6, ETV4, SPRY2, SPRY4, PHLDA1, ETV5, DUSP4, CCND1, EPHA2, and/or EPHA4) is determined in tumor cells.

In certain instances, a reference level, reference sample, reference cell, reference tissue, control sample, control cell, or control tissue is a single sample or a combination of multiple samples from the same subject or individual that are obtained at one or more different time points than when the test sample is obtained. For example, a reference level, reference sample, reference cell, reference tissue, control sample, control cell, or control tissue is obtained at an earlier time point from the same subject or individual than when the test sample is obtained. Such reference level, reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference level, reference sample, reference cell, reference tissue, control sample, control cell, or control tissue is a combination of multiple samples from one or more healthy individuals who are not the patient. In certain embodiments, a reference level, reference sample, reference cell, reference tissue, control sample, control cell, or control tissue is a combination of multiple samples from one or more individuals with a disease or disorder (e.g., cancer) who are not the patient or individual. In certain embodiments, a reference level, reference sample, reference cell, reference tissue, control sample, control cell, or control tissue is pooled RNA samples from normal tissues or pooled plasma or serum samples from one or more individuals who are not the patient. In certain embodiments, a reference level, reference sample, reference cell, reference tissue, control sample, control cell, or control tissue is pooled RNA samples from tumor tissues or pooled plasma or serum samples from one or more individuals with a disease or disorder (e.g., cancer) who are not the patient. In certain embodiments, the reference level is the median level of expression of a biomarker across a set of samples (e.g., a set of tissue samples (e.g., a set of tumor tissue samples)). In certain embodiments, the reference level is the median level of expression of a biomarker across a population of patients having a particular disease or disorder (e.g., a proliferative cell disorder (e.g., a cancer)).

In some embodiments of any of the methods, elevated or increased expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art-known methods such as those described herein, as compared to a reference level, reference sample, reference cell, reference tissue, control sample, control cell, or control tissue. In certain embodiments, the elevated or increased expression refers to the increase in expression level (amount) of a biomarker in the sample, wherein the increase is at least about any of 1.5×, 1.75×, 2×, 3×, 4×, 5×, 6×, 7×, 8×, 9×, 10×, 25×, 50×, 75×, or 100× the expression level (amount) of the respective biomarker in a reference level, reference sample, reference cell, reference tissue, control sample, control cell, or control tissue. In some embodiments, elevated expression refers to an overall increase of greater than about 1.5-fold, about 1.75-fold, about 2-fold, about 2.25-fold, about 2.5-fold, about 2.75-fold, about 3.0-fold, or about 3.25-fold as compared to a reference sample, reference cell, reference tissue, control sample, control cell, control tissue, or internal control (e.g., housekeeping gene).

In some embodiments of any of the methods, reduced or decreased expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference level, reference sample, reference cell, reference tissue, control sample, control cell, or control tissue. In certain embodiments, reduced expression refers to the decrease in expression level (amount) of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level (amount) of the respective biomarker in a reference level, reference sample, reference cell, reference tissue, control sample, control cell, or control tissue.

The invention also provides methods of using a MAPK activity score to inform diagnosis and/or treatment in connection with the methods described herein. In one embodiment, a MAPK activity score is determined according to the algorithm:

$$\frac{Σ {z}_{i}}{\sqrt{n}} ,$$

where zᵢ is the z-score or eacn gene, normalized across all samples or to a set of housekeeping genes, and n is the number of genes included in the set. In some instances, the genes included in the set used to determine a MAPK activity score are one or more of DUSP6, ETV4, SPRY2, SPRY4, PHLDA1, ETV5, DUSP4, CCND1, EPHA2, and EPHA4. For example, the genes comprising the set used to determine a MAPK activity score may be DUSP6, ETV4, SPRY2, SPRY4, PHLDA1, ETV5, DUSP4, CCND1, EPHA2, and EPHA4. The MAPK activity score may be determined across a population of samples (e.g., tissue samples (e.g., tumor tissue samples)). In some instances, the median MAPK activity score across a population of samples represents the MAPK activity score across a population of patients suffering from a particular cancer (e.g., lung cancer, skin cancer, breast cancer, stomach cancer). In some instances, the median MAPK activity score is a previously defined MAPK activity score for the cancer. The previously defined median MAPK activity score can be determined, for example, from a plurality (e.g., at least 100) of samples (e.g., archived samples) from patients having the cancer. A MAPK activity score greater than the median MAPK activity score is a high MAPK activity score (MAPK-high) and may identify a patient who is likely to benefit from treatment including one or more MAPK signaling inhibitors. In some instances, the high MAPK activity score is greater than 1% or more (e.g., 2%, 3%, 4%, 5%, 6%, 7%, 8%, 9% 10%, 20%, 30%, 40%, 50%, 60%, 70%, 80%, 90% or more) of the median MAPK activity score. In some instances, a MAPK activity score less than the median MAPK activity score is a low MAPK activity score (MAPK-low) and may identify a patient with a reduced likelihood of benefit from treatment including one of more MAPK signaling inhibitors. In some instances, the low MAPK activity score is less than 1% or more (e.g., 2%, 3%, 4%, 5%, 6%, 7%, 8%, 9% 10%, 20%, 30%, 40%, 50%, 60%, 70%, 80%, 90% or more) of the median MAPK activity score.

In some instances, the MAPK activity score can be combined with the determination of a baseline gene signature (e.g., a cell cycle or immune gene signature). In some instances, the baseline gene signature can be determined by clustering whole-transcriptome RNA-Seq expression data from multiple samples (e.g., a group of patient samples (e.g., a group of cancer patient samples)) of genes known in the art to be involved in proliferation into a cell cycle baseline gene signature subgroup. In some instances, whole-transcriptome RNA-Seq expression data from multiple samples (e.g., a group of patient samples (e.g., a group of cancer patient samples)) of genes known in the art to be expressed on immune cells can be clustered into an immune baseline gene signature subgroup. In some instances, a “Cell Cycle/MAPK-high” determination identifies a patient with an increased likelihood of benefit from treatment including one or more MAPK signaling inhibitors relative to a “Cell Cycle/MAPK-low” determination. In some instances, an “Immune/MAPK high” determination identifies a patient with an increased likelihood of benefit from treatment including one or more MAPK signaling inhibitors relative to an “Immune/MAPK-low” determination. In some instances, the determination of a cell cycle signature identifies a patient with an increased likelihood of responding to treatment comprising a MAPK signaling inhibitor, including a combination of a MEK inhibitor and a BRAF inhibitor, such as a combination of cobimetinib and vemurafenib.

B. Treatment with MAPK Signaling Inhibitors

The present invention provides methods for treating a patient having a cancer (e.g., lung cancer, breast cancer, skin cancer, colorectal cancer, stomach cancer, lymphoid cancer, ovarian cancer, and cervical cancer). In some instances, the methods of the invention include administering to the patient a MAPK signaling inhibitor. Any of the MAPK signaling inhibitors described herein or known in the art may be used in connection with the methods. In some instances, the methods involve determining the expression level of DUSP6, ETV4, SPRY2, SPRY4, PHLDA1, ETV5, DUSP4, CCND1, EPHA2, and/or EPHA4 in a sample obtained from a patient and administering a therapy including one or more MAPK signaling inhibitors to the patient based an increased expression level of DUSP6, ETV4, SPRY2, SPRY4, PHLDA1, ETV5, DUSP4, CCND1, EPHA2, and/or EPHA4 in the sample as compared to a reference level. In some instances, administering a MAPK signaling inhibitor is after the expression level of at least one of DUSP6, ETV4, SPRY2, SPRY4, PHLDA1, ETV5, DUSP4, CCND1, EPHA2, and/or EPHA4 has been determined to be increased relative to a reference level. In some instances, a patient currently being treated with a MAPK signaling inhibitor may continue to receive treatment including a MAPK signaling inhibitor following a determination that the expression level of at least one of DUSP6, ETV4, SPRY2, SPRY4, PHLDA1, ETV5, DUSP4, CCND1, EPHA2, and/or EPHA4 is increased relative to a reference level.

In any of the preceding methods, one or more MAPK signaling inhibitors may be administered when the expression level of at least one of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least one gene. In some instances, the expression levels of at least two of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least two genes. In some instances, the expression level of at least three of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient has been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least three genes. In some instances, the expression level of at least four of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least four genes. In some instances, the expression level of at least five of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least five genes. In some instances, the expression level of at least six of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least six genes. In some instances, the expression level of at least seven of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least seven genes. In some instances, the expression level of at least eight of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least eight genes. In some instances, the expression level of at least nine of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the at least nine genes. In some instances, the expression level of all ten of DUSP6, ETV4, SPRY2, SPRY4, PHLDA1, ETV5, DUSP4, CCND1, EPHA2, and EPHA4 in the sample (e.g., a tissue sample (e.g., a tumor tissue sample)) obtained from the patient have been determined to have changed (e.g., increased) by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, or about 50% or more) relative to a reference level of the ten genes.

In certain embodiments, the method includes administering to a lung cancer patient a MAPK signaling inhibitor when an increased level of expression of PHLDA1, EPHA2, CCND1, SPRY2, SPRY4, ETV4, DUSP4, and/or DUSP6 relative to a reference level identifies the patient as having an increased likelihood of benefit from treatment with a MAPK signaling inhibitor. In some instances, the method includes administering to a breast cancer patient a MAPK signaling inhibitor when an increased level of expression of PHLDA1, SPRY2, ETV4, EPHA2, ETV5, and/or SPRY4 relative to a reference level identifies the patient as having an increased likelihood of benefit from treatment with a MAPK signaling inhibitor. In some instances, the method includes administering to a skin cancer patient a MAPK signaling inhibitor when an increased level of expression of DUSP4, SPRY4, and/or ETV4 relative to a reference level identifies the patient as having an increased likelihood of benefit from treatment with a MAPK signaling inhibitor. In some instances, the method includes administering to a colorectal cancer patient a MAPK signaling inhibitor when an increased level of expression of PHLDA1, DUSP6, SPRY4, and/or SPRY2 relative to a reference level identifies the patient as having an increased likelihood of benefit from treatment with a MAPK signaling inhibitor. In some instances, the method includes administering to a stomach cancer patient a MAPK signaling inhibitor when an increased level of expression of DUSP4 relative to a reference level identifies the patient as having an increased likelihood of benefit from treatment with a MAPK signaling inhibitor. In some instances, the method includes administering to lymphoid cancer patient a MAPK signaling inhibitor when an increased level of expression of DUSP6, ETV5, SPRY2, SPRY4, and/or ETV4 relative to a reference level identifies the patient having an increased likelihood of benefit from treatment with a MAPK signaling inhibitor. In some instances, the method includes administering to an ovarian cancer patient a MAPK signaling inhibitor when an increased level of expression of SPRY2 and/or DUSP6 relative to a reference level identifies the patient having an increased likelihood of benefit from treatment with a MAPK signaling inhibitor. In some instances, the method includes administering to a cervical cancer patient a MAPK signaling inhibitor when an increased level of expression of DUSP6 relative to a reference level identifies the patient having an increased likelihood of benefit from treatment with a MAPK signaling inhibitor.

The invention further provides a method of treating a patient having a cancer, including administering to the patient a therapeutically effective amount of one or more MAPK signaling inhibitors, based on the determination of a high MAPK activity score from a tumor sample obtained from the patient. The invention further provides a method of treating a patient having a cancer including administering to the patient a therapeutically effective amount one or more MAPK signaling inhibitors based on the determination of a Cell Cycle/MAPK-high activity score from a tumor sample obtained from the patient. A MAPK activity score is determined according to the algorithm:

$$\frac{Σ {z}_{i}}{\sqrt{n}} ,$$

where zᵢ is the z-score of each gene, normalized across all samples or to a set of housekeeping genes, and n is the number of genes included in the set. In some instances, the genes including the set used to determine a MAPK activity score are one or more of DUSP6, ETV4, SPRY2, SPRY4, PHLDA1, ETV5, DUSP4, CCND1, EPHA2, and EPHA4. For example, the genes comprising the set used to determine a MAPK activity score may be DUSP6, ETV4, SPRY2, SPRY4, PHLDA1, ETV5, DUSP4, CCND1, EPHA2, and EPHA4. The MAPK activity score may be determined across a population of samples (e.g., tissue samples (e.g., tumor tissue samples)). In some instances, the median MAPK activity score across a population of samples represents the MAPK activity score across a population of patients suffering from a particular cancer (e.g., lung cancer, skin cancer, breast cancer, stomach cancer). A MAPK activity score greater than the median MAPK activity score is a high MAPK activity score (MAPK-high) and may identify a patient who is likely to benefit from treatment including one or more MAPK signaling inhibitors. In some instances, the high MAPK activity score is greater than 1% or more (e.g., 2%, 3%, 4%, 5%, 6%, 7%, 8%, 9% 10%, 20%, 30%, 40%, 50%, 60%, 70%, 80%, 90% or more) of the median MAPK activity score. In some instances, a MAPK activity score less than the median MAPK activity score is a low MAPK activity score (MAPK-low) and may identify a patient with a reduced likelihood of benefit from treatment including one of more MAPK signaling inhibitors. In some instances, the low MAPK activity score is less than 1% or more (e.g., 2%, 3%, 4%, 5%, 6%, 7%, 8%, 9% 10%, 20%, 30%, 40%, 50%, 60%, 70%, 80%, 90% or more) of the median MAPK activity score. In some instances, the MAPK activity score can be combined with the determination of a baseline gene signature (e.g., a cell cycle or immune gene signature). In some instances, a “Cell Cycle/MAPK-high” determination identifies a patient with an increased likelihood of benefit from treatment including one or more MAPK signaling inhibitors relative to a “Cell Cycle/MAPK-low” determination. In some instances, an “Immune/MAPK-high” determination identifies a patient with an increased likelihood of benefit from treatment including one or more MAPK signaling inhibitors relative to an “Immune/MAPK-low” determination.

In some instances, a MAPK activity score is determined before administration of a MAPK signaling inhibitor. In some instances, a patient currently being treated with a MAPK signaling inhibitor may continue to receive treatment including a MAPK signaling inhibitor following the determination of a high MAPK activity score. In some instances, a combination MAPK signaling inhibitors (e.g., a combination of a MEK inhibitor and a BRAF inhibitor, such as a combination of cobimetinib and vemurafenib) is administered to a patient who has been determined to have a cell cycle signature and identified as one who has an increased likelihood of responding to treatment including two or more MAPK signaling inhibitors.

In any of the above methods, administration of one or more MAPK signaling inhibitor can have the therapeutic effect (i.e., benefit) of a cellular or biological response, a complete response, a partial response, a stable disease (without progression or relapse), or a response with a later relapse of the patient from or as a result of the treatment with the MAPK signaling inhibitor. For example, an effective response can be reduced tumor size (volume), increased progression-free survival (PFS), and/or increased overall survival (OS) in a patient diagnosed as expressing a higher level of one or more of the biomarkers (e.g., DUSP6, ETV4, SPRY2, SPRY4, PHLDA1, ETV5, DUSP4, CCND1, EPHA2, and EPHA4) compared to a reference level (including, e.g., the median expression level of the biomarker in a sample from a group/population of patients being tested; the median expression level of the biomarker in a sample from a group/population of patients having a particular cancer; the level in a sample previously obtained from the individual at a prior time; or the level in a sample from a patient who received prior treatment with a MAPK signaling inhibitor). In some instances, administration of a MAPK signaling inhibitor has a therapeutic effect of a reduction in tumor size (volume) by 1% or more (e.g., 2%, 3%, 4%, 5%, 6%, 7%, 8%, 9% 10%, 20%, 30%, 40%, 50%, 60%, 70%, 80%, or 90% or more). The increased expression at least one of the biomarker (e.g., DUSP6, ETV4, SPRY2, SPRY4, PHLDA1, ETV5, DUSP4, CCND1, EPHA2, and EPHA4) predicts such therapeutic efficacy. In some instances, administration of a MAPK signaling inhibitor has the therapeutic effect of increasing progression-free survival (PFS) by 1 day or more (e.g., by 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, or 1 year or more).

MAPK Signaling Inhibitors for Use in the Methods of the Invention

Provided herein are methods for treating or delaying the progression of a proliferative cell disorder (e.g., cancer (e.g., lung cancer, breast cancer, skin cancer, colorectal cancer, stomach cancer, lymphoid cancer, ovarian cancer, and cervical cancer)) in a patient comprising administering to the patient a therapeutically effective amount of one or more MAPK signaling inhibitors.

A MAPK signaling inhibitor is a molecule that decreases, blocks, inhibits, abrogates, or interferes with signal transduction through the MAPK pathway (e.g., the RAS/RAF/MEK/ERK pathway). In some embodiments, a MAPK signaling inhibitor may inhibit the activity of one or more proteins involved in the activation of MAPK signaling. In some embodiments, a MAPK signaling inhibitor may activate the activity of one or more proteins involved in the inhibition of MAPK signaling. MAPK signaling inhibitors include, but are not limited to, MEK inhibitors, BRAF inhibitors, ERK inhibitors, CRAF inhibitors, and RAF inhibitors. In some embodiments, a MAPK signaling inhibitor is a small molecule. In some embodiments, the MAPK signaling inhibitor may be a protein (e.g., a peptide). In some embodiments, the MAPK signaling inhibitor may be an antibody, an antigen binding fragment thereof, an immunoadhesin, a fusion protein, or oligopeptide.

Examples of BRAF inhibitors that may be used in accordance with the invention include, without limitation, vemurafenib (ZELBORAF®), dabrafenib, encorafenib (LGX818), GDC-0879, XL281, ARQ736, PLX3603, RAF265, and sorafenib, and pharmaceutically acceptable salts thereof. BRAF inhibitors may inhibit only BRAF or may inhibit BRAF and one or more additional targets. Preferred BRAF inhibitors as described in PCT Application Publication Nos. WO 2005/062795, WO 2007/002325, WO 2007/002433, WO 2008/079903, and WO 2008/079906, which are each incorporated herein by reference in its entirety. Examples of ERK inhibitors that may be used in accordance with the invention include, without limitation, ravoxertinib (GDC-0994) and ulixertinib (BVD-523), and pharmaceutically acceptable salts (e.g., a besylate salt (e.g., a besylate salt of ravoxertinib)) thereof. ERK inhibitors may inhibit only ERK or may inhibit ERK and one or more additional targets. Preferred ERK inhibitors as described in PCT Application Publication Nos. WO 2013/130976, WO 2012/118850, WO 2013/020062, WO 2015/154674, WO 2015/085007, WO 2015/032840, WO 2014/036015, WO 2014/060395, WO 2015/103137, and WO 2015/103133, which are each incorporated herein by reference in its entirety.

Examples of MEK inhibitors that may be used in accordance with the invention include, without limitation, cobimetinib (e.g., cobimetinib hemifumarate; COTELLIC®), trametinib, binimetinib, selumetinib, pimasertinib, refametinib, GDC-0623, PD-0325901, and BI-847325, and pharmaceutically acceptable salts thereof. MEK inhibitors may inhibit only MEK or may inhibit MEK and one or more additional targets. Preferred MEK inhibitors as described in PCT Application Publication Nos. WO 2007/044515, WO 2008/024725, WO 2008/024724, WO 2008/067481, WO 2008/157179, WO 2009/085983, WO 2009/085980, WO 2009/082687, WO 2010/003025, and WO 2010/003022, which are each incorporated herein by reference in its entirety.

Examples of CRAF inhibitors that may be used in accordance with the invention include, without limitation, sorafenib, and pharmaceutically acceptable salts thereof. CRAF inhibitors may inhibit only CRAF or may inhibit CRAF and one or more additional targets.

Dosage and Administration

Once a patient responsive or sensitive to treatment with a MAPK signaling inhibitor has been identified, treatment with the MAPK signaling inhibitor, alone or in combination with other therapeutic agents, can be carried out. Such treatment may result in, for example, a reduction in tumor size or an increase in progression-free survival (PFS) and/or overall survival (OS). Moreover, treatment with the combination of a MAPK signaling inhibitor and at least one additional therapeutic agent preferably results in an additive, more preferably synergistic (or greater than additive), therapeutic benefit to the patient. Preferably, in this combination method the timing between at least one administration of the MAPK signaling inhibitor and at least one additional therapeutic agent is about one month or less, and more preferably, about two weeks or less.

It will be appreciated by those of skill in the art that the exact manner of administering a therapeutically effective amount of a MAPK signaling inhibitor to a patient following diagnosis of their likely responsiveness to the MAPK signaling inhibitor will be at the discretion of the attending physician. The mode of administration, including dosage, combination with other agents, timing and frequency of administration, and the like, may be affected by the diagnosis of a patient's likely responsiveness to such MAPK signaling inhibitor, as well as the patient's condition and history. Thus, even patients having cancers who are predicted to be relatively insensitive to a MAPK signaling inhibitor may still benefit from treatment therewith, particularly in combination with other agents, including agents that may alter a patient's responsiveness to the antagonist.

A composition comprising a MAPK signaling inhibitor will be formulated, dosed, and administered in a fashion consistent with good medical practice. Factors for consideration in this context include the particular type of cancer being treated (e.g., lung cancer, breast cancer, skin cancer, colorectal cancer, stomach cancer, lymphoid cancer, ovarian cancer, and cervical cancer), the particular mammal being treated (e.g., human), the clinical condition of the individual patient, the cause of the cancer, the site of delivery of the agent, possible side-effects, the type of inhibitor, the method of administration, the scheduling of administration, and other factors known to medical practitioners. The effective amount of the MAPK signaling inhibitor to be administered will be governed by such considerations.

A physician having ordinary skill in the art can readily determine and prescribe the effective amount of the pharmaceutical composition required, depending on such factors as the particular antagonist type. For example, the physician could start with doses of such a MAPK signaling inhibitor, employed in the pharmaceutical composition at levels lower than that required in order to achieve the desired therapeutic effect and gradually increase the dosage until the desired effect is achieved. The effectiveness of a given dose or treatment regimen of the antagonist can be determined, for example, by assessing signs and symptoms in the patient using standard measures of efficacy.

In certain examples, the MAPK signaling inhibitor may be the only agent administered to the subject (i.e., as a monotherapy).

In certain examples, the patient is treated with the same MAPK signaling inhibitor at least twice. Thus, the initial and second MAPK signaling inhibitor exposures are preferably with the same inhibitor, and more preferably all MAPK signaling inhibitor exposures are with the same MAPK signaling inhibitor, i.e., treatment for the first two exposures, and preferably all exposures, is with one type of MAPK signaling inhibitor.

Treatment with MAPK signaling inhibitors, or pharmaceutically acceptable salts thereof, can be carried out according to standard methods. Exemplary methods for administration of cobimetinib (e.g., cobimetinib fumarate (COTELLIC®)) are described in Prescribing Information for cobimetinib fumarate (COTELLIC®) in the United States, Genentech, Inc. (Nov. 10, 2015), which is incorporated herein by reference in its entirety. Exemplary methods for the administration of vemurafenib (ZELBORAF®) are described in Prescribing Information for vemurafenib (ZELBORAF®) in the United States, Hoffmann La Roche, Inc. (Aug. 11, 2015), which is incorporated herein by reference in its entirety.

If multiple exposures of a MAPK signaling inhibitor are provided, each exposure may be provided using the same or a different administration means. In one embodiment, each exposure is given by oral administration. In one embodiment, each exposure is by intravenous administration. In another embodiment, each exposure is given by subcutaneous administration. In yet another embodiment, the exposures are given by both intravenous and subcutaneous administration.

The duration of therapy can be continued for as long as medically indicated or until a desired therapeutic effect (e.g., those described herein) is achieved. In certain embodiments, the therapy is continued for 1 month, 2 months, 4 months, 6 months, 8 months, 10 months, 1 year, 2 years, 3 years, 4 years, 5 years, or for a period of years up to the lifetime of the subject.

As noted above, however, these suggested amounts of MAPK signaling inhibitors are subject to a great deal of therapeutic discretion. The key factor in selecting an appropriate dose and scheduling is the result obtained, as indicated above. In some embodiments, the MAPK signaling inhibitor is administered as close to the first sign, diagnosis, appearance, or occurrence of the proliferative cell disorder (e.g., cancer) as possible.

1. Routes of Administration

MAPK signaling inhibitors and any additional therapeutic agents may be formulated, dosed, and administered in a fashion consistent with good medical practice. Factors for consideration in this context include the particular disorder being treated (e.g., cancer), the particular mammal being treated, the clinical condition of the individual patient, the cause of the disorder, the site of delivery of the agent, the method of administration, the scheduling of administration, and other factors known to medical practitioners. The MAPK signaling inhibitor need not be, but is optionally formulated with and/or administered concurrently with, one or more agents currently used to prevent or treat the disorder in question (e.g., cancer).

For the prevention or treatment of a cancer, the appropriate dosage of a MAPK signaling inhibitor described herein (when used alone or in combination with one or more other additional therapeutic agents) will depend on the type of disease to be treated, the severity and course of the disease, whether the MAPK signaling inhibitor is administered for preventive or therapeutic purposes, previous therapy, the patient's clinical history and response to the MAPK signaling inhibitor, and the discretion of the attending physician. The MAPK signaling inhibitor is suitably administered to the patient at one time or over a series of treatments. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the MAPK signaling inhibitor). An initial higher loading dose, followed by one or more lower doses may be administered. However, other dosage regimens may be useful. The progress of this therapy is easily monitored by conventional techniques and assays.

The MAPK signaling inhibitor can be administered by any suitable means, including orally, parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated. In addition, the MAPK signaling inhibitor may suitably be administered by pulse infusion, e.g., with declining doses of the MAPK signaling inhibitor. Most preferably, the dosing is given by oral administration.

If multiple exposures of a MAPK signaling inhibitor are provided, each exposure may be provided using the same or a different administration means. In one embodiment, each exposure is by oral administration. For example, one or more MAPK signaling inhibitors, such as cobimetinib, vemurafenib, and/or ravoxertinib, can provided in tablet form. For example, one or more MAPK signaling inhibitors, such as cobimetinib, vemurafenib, and/or ravoxertinib, can be administered twice a day. In another embodiment, each exposure is given intravenously (i.v.). In another embodiment, each exposure is given by subcutaneous (s.c.) administration. In yet another embodiment, the exposures are given by both i.v. and s.c. administration.

2. Combination Therapy

The methods may further involve administering to the patient an effective amount of a MAPK signaling inhibitor in combination with an additional therapeutic agent. In some instances, the additional therapeutic agent is an additional MAPK signaling inhibitor. In some instances, the additional therapeutic agent is an anti-cancer agent, such as a chemotherapeutic agent, a growth-inhibitory agent, a biotherapy, an immunotherapy, or a radiation therapy agent. In addition, cytotoxic agents, anti-angiogenic, and anti-proliferative agents can be used in combination with the MAPK signaling inhibitor. In some instances, the MAPK signaling inhibitor is used in combination with an anti-cancer therapy, such as surgery.

The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially. In general, during alternation therapy, an effective dosage of each active ingredient is administered sequentially (i.e., serially), whereas in combination therapy, effective dosages of two or more active ingredients are administered together.

As described above, the therapeutic methods may include administering a combination of two or more (e.g., three or more) MAPK signaling inhibitors. In some instances, a MEK inhibitor is administered in combination with at least one BRAF inhibitor. In some instances, a MEK inhibitor is administered in combination with at least one ERK inhibitor. In some instances, a MEK inhibitor is administered in combination with at least one CRAF inhibitor. In some instances, a MEK inhibitor is administered in combination with at least one RAF inhibitor. In some instances, a MEK inhibitor is administered with at least one RAS inhibitor. In some instances, a MEK inhibitor is administered with at least one KRAS inhibitor. In some instances, a BRAF inhibitor is administered in combination with at least one MEK inhibitor. In some instances, a BRAF inhibitor is administered in combination with at least one ERK inhibitor. In some instances, a BRAF inhibitor is administered in combination with at least one CRAF inhibitor. In some instances, a BRAF inhibitor is administered in combination with at least one RAF inhibitor. In some instances, a BRAF inhibitor is administered with at least one RAS inhibitor. In some instances, a BRAF inhibitor is administered with at least one KRAS inhibitor. In some instances, an ERK inhibitor is administered in combination with at least one MEK inhibitor. In some instances, an ERK inhibitor is administered in combination with at least one BRAF inhibitor. In some instances, an ERK inhibitor is administered in combination with at least one CRAF inhibitor. In some instances, an ERK inhibitor is administered in combination with at least one RAF inhibitor. In some instances, an ERK inhibitor is administered with at least one RAS inhibitor. In some instances, an ERK inhibitor is administered with at least one KRAS inhibitor. In some instances, a CRAF inhibitor is administered in combination with at least one MEK inhibitor. In some instances, a CRAF inhibitor is administered in combination with at least one ERK inhibitor. In some instances, a CRAF inhibitor is administered in combination with at least one RAF inhibitor. In some instances, a CRAF inhibitor is administered in combination with at least one BRAF inhibitor. In some instances, a CRAF inhibitor is administered with at least one RAS inhibitor. In some instances, a CRAF inhibitor is administered with at least one KRAS inhibitor. In some instances, a RAF inhibitor is administered in combination with at least one MEK inhibitor. In some instances, a RAF inhibitor is administered in combination with at least one ERK inhibitor. In some instances, a RAF inhibitor is administered in combination with at least one CRAF inhibitor. In some instances, a RAF inhibitor is administered in combination with at least one BRAF inhibitor. In some instances, a RAF inhibitor is administered with at least one RAS inhibitor. In some instances, a RAF inhibitor is administered with at least one KRAS inhibitor.

The methods may also involve administering to the patient an effective amount of a MAPK signaling inhibitor in combination with a chemotherapeutic agent, such as cyclophosphamide, hydroxydaunorubicin, adriamycin, doxorubincin, vincristine (ONCOVIN™), prednisolone, CHOP, CVP, or COP. In another embodiment, the combination includes docetaxel, doxorubicin, and cyclophosphamide.

In other instances, the method includes administering a MAPK signaling inhibitor in combination with an immunotherapeutic, such as a therapeutic antibody. In one embodiment, the therapeutic antibody is an antibody that binds a cancer cell surface marker or tumor associated-antigen (TAA). In one embodiment, the therapeutic antibody is an anti-HER2 antibody, trastuzumab (e.g., HERCEPTIN®). In one embodiment, the therapeutic antibody is an anti-HER2 antibody, pertuzumab (OMNITARG™). In another embodiment, the therapeutic antibody either a naked antibody or an antibody-drug conjugate (ADC).

Without wishing to be bound to theory, it is thought that enhancing T-cell stimulation, by promoting an activating co-stimulatory molecule or by inhibiting a negative co-stimulatory molecule, may promote tumor cell death thereby treating or delaying progression of cancer. Therefore, in some instances, a MAPK signaling inhibitor may be administered in conjunction with an agonist directed against an activating co-stimulatory molecule. In some instances, an activating co-stimulatory molecule may include CD40, CD226, CD28, OX40, GITR, CD137, CD27, HVEM, or CD127. In some instances, the agonist directed against an activating co-stimulatory molecule is an agonist antibody that binds to CD40, CD226, CD28, OX40, GITR, CD137, CD27, HVEM, or CD127. In some instances, a MAPK signaling inhibitor may be administered in conjunction with an antagonist directed against an inhibitory co-stimulatory molecule. In some instances, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some instances, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some instances, a MAPK signaling inhibitor may be administered in conjunction with an antagonist directed against CTLA-4 (also known as CD152), e.g., a blocking antibody. In some instances, a MAPK signaling inhibitor may be administered in conjunction with ipilimumab (also known as MDX-010, MDX-101, or YERVOY®). In some instances, a MAPK signaling inhibitor may be administered in conjunction with tremelimumab (also known as ticilimumab or CP-675,206). In some instances, a MAPK signaling inhibitor may be administered in conjunction with an antagonist directed against B7-H3 (also known as CD276), e.g., a blocking antibody. In some instances, a MAPK signaling inhibitor may be administered in conjunction with MGA271. In some instances, a MAPK signaling inhibitor may be administered in conjunction with an antagonist directed against a TGF-beta, e.g., metelimumab (also known as CAT-192), fresolimumab (also known as GC1008), or LY2157299.

In some instances, a MAPK signaling inhibitor may be administered in conjunction with a treatment including adoptive transfer of a T-cell (e.g., a cytotoxic T-cell or CTL) expressing a chimeric antigen receptor (CAR). In some instances, a MAPK signaling inhibitor may be administered in conjunction with a treatment including adoptive transfer of a T-cell including a dominant-negative TGF beta receptor, e.g., a dominant-negative TGF beta type II receptor. In some instances, a MAPK signaling inhibitor may be administered in conjunction with a treatment including a HERCREEM protocol (see, e.g., ClinicalTrials.gov Identifier NCT00889954).

In some instances, a MAPK signaling inhibitor may be administered in conjunction with an agonist directed against CD137 (also known as TNFRSF9, 4-1BB, or ILA), e.g., an activating antibody. In some instances, a MAPK signaling inhibitor may be administered in conjunction with urelumab (also known as BMS-663513). In some instances, a MAPK signaling inhibitor may be administered in conjunction with an agonist directed against CD40, e.g., an activating antibody. In some instances, a MAPK signaling inhibitor may be administered in conjunction with CP-870893. In some instances, a MAPK signaling inhibitor may be administered in conjunction with an agonist directed against OX40 (also known as CD134), e.g., an activating antibody. In some instances, a MAPK signaling inhibitor may be administered in conjunction with an anti-OX40 antibody (e.g., AgonOX). In some instances, a MAPK signaling inhibitor may be administered in conjunction with an agonist directed against CD27, e.g., an activating antibody. In some instances, a MAPK signaling inhibitor may be administered in conjunction with CDX-1127. In some instances, a MAPK signaling inhibitor may be administered in conjunction with an antagonist directed against indoleamine-2,3-dioxygenase (IDO). In some instances, with the IDO antagonist is 1-methyl-D-tryptophan (also known as 1-D-MT). In some instances, a MAPK signaling inhibitor may be administered in conjugation with a PD-1 axis binding antagonist. In some instances, the PD-1 axis binding antagonist is a PD-L1 antibody.

In some instances, a MAPK signaling inhibitor may be administered in conjunction with an antibody-drug conjugate. In some instances, the antibody-drug conjugate comprises mertansine or monomethyl auristatin E (MMAE). In some instances, a MAPK signaling inhibitor may be administered in conjunction with an anti-NaPi2b antibody-MMAE conjugate (also known as DNIB0600A or RG7599). In some instances, a MAPK signaling inhibitor may be administered in conjunction with trastuzumab emtansine (also known as T-DM1, ado-trastuzumab emtansine, or KADCYLA®, Genentech). In some instances, a MAPK signaling inhibitor may be administered in conjunction with DMUC5754A. In some instances, a MAPK signaling inhibitor may be administered in conjunction with an antibody-drug conjugate targeting the endothelin B receptor (EDNBR), e.g., an antibody directed against EDNBR conjugated with MMAE.

In some instances, a MAPK signaling inhibitor may be administered in conjunction with an anti-angiogenesis agent. In some instances, a MAPK signaling inhibitor may be administered in conjunction with an antibody directed against a VEGF, e.g., VEGF-A. In some instances, a MAPK signaling inhibitor may be administered in conjunction with bevacizumab (also known as AVASTIN®, Genentech). In some instances, a MAPK signaling inhibitor may be administered in conjunction with an antibody directed against angiopoietin 2 (also known as Ang2). In some instances, a MAPK signaling inhibitor may be administered in conjunction with MEDI3617. In some instances, a MAPK signaling inhibitor may be administered in conjunction with an antineoplastic agent. In some instances, a MAPK signaling inhibitor may be administered in conjunction with an agent targeting CSF-1R (also known as M-CSFR or CD115). In some instances, a MAPK signaling inhibitor may be administered in conjunction with anti-CSF-1R (also known as IMC-CS4). In some instances, a MAPK signaling inhibitor may be administered in conjunction with an interferon, for example interferon alpha or interferon gamma. In some instances, a MAPK signaling inhibitor may be administered in conjunction with Roferon-A (also known as recombinant Interferon alpha-2a). In some instances, a MAPK signaling inhibitor may be administered in conjunction with GM-CSF (also known as recombinant human granulocyte macrophage colony stimulating factor, rhu GM-CSF, sargramostim, or LEUKINE®). In some instances, a MAPK signaling inhibitor may be administered in conjunction with IL-2 (also known as aldesleukin or PROLEUKIN®). In some instances, a MAPK signaling inhibitor may be administered in conjunction with IL-12. In some instances, a MAPK signaling inhibitor may be administered in conjunction with an antibody targeting CD20. In some instances, the antibody targeting CD20 is obinutuzumab (also known as GA101 or GAZYVA®) or rituximab. In some instances, a MAPK signaling inhibitor may be administered in conjunction with an antibody targeting GITR. In some instances, the antibody targeting GITR is TRX518.

In some instances, a MAPK signaling inhibitor may be administered in conjunction with a cancer vaccine. In some instances, the cancer vaccine is a peptide cancer vaccine, which in some instances is a personalized peptide vaccine. In some instances the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a MAPK signaling inhibitor may be administered in conjunction with an adjuvant. In some instances, a MAPK signaling inhibitor may be administered in conjunction with a treatment including a TLR agonist, e.g., Poly-ICLC (also known as HILTONOL®), LPS, MPL, or CpG ODN. In some instances, a MAPK signaling inhibitor may be administered in conjunction with tumor necrosis factor (TNF) alpha. In some instances, a MAPK signaling inhibitor may be administered in conjunction with IL-1. In some instances, a MAPK signaling inhibitor may be administered in conjunction with HMGB1. In some instances, a MAPK signaling inhibitor may be administered in conjunction with an IL-10 antagonist. In some instances, a MAPK signaling inhibitor may be administered in conjunction with an IL-4 antagonist. In some instances, a MAPK signaling inhibitor may be administered in conjunction with an IL-13 antagonist. In some instances, a MAPK signaling inhibitor may be administered in conjunction with an HVEM antagonist. In some instances, a MAPK signaling inhibitor may be administered in conjunction with an ICOS agonist, e.g., by administration of ICOS-L, or an agonistic antibody directed against ICOS. In some instances, a MAPK signaling inhibitor may be administered in conjunction with a treatment targeting CX3CL1. In some instances, a MAPK signaling inhibitor may be administered in conjunction with a treatment targeting CXCL9. In some instances, a MAPK signaling inhibitor may be administered in conjunction with a treatment targeting CXCL10. In some instances, a MAPK signaling inhibitor may be administered in conjunction with a treatment targeting CCLS. In some instances, a MAPK signaling inhibitor may be administered in conjunction with an LFA-1 or ICAM1 agonist. In some instances, a MAPK signaling inhibitor may be administered in conjunction with a Selectin agonist.

In general, for the prevention or treatment of disease, the appropriate dosage of the additional therapeutic agent will depend on the type of disease to be treated, the type of antibody, the severity and course of the disease, whether the MAPK signaling inhibitor and additional agent (e.g., TMZ) are administered for preventive or therapeutic purposes, previous therapy, the patient's clinical history and response to the MAPK signaling inhibitor and additional agent, and the discretion of the attending physician. The MAPK signaling inhibitor and additional agent are suitably administered to the patient at one time or over a series of treatments. The MAPK signaling inhibitor is typically administered as set forth above. Depending on the type and severity of the disease, about 20 mg/m² to 600 mg/m² of the additional agent is an initial candidate dosage for administration to the patient, whether, for example, by one or more separate administrations, or by continuous infusion. One typical daily dosage might range from about or about 20 mg/m², 85 mg/m², 90 mg/m², 125 mg/m², 200 mg/m², 400 mg/m², 500 mg/m² or more, depending on the factors mentioned above. For repeated administrations over several days or longer, depending on the condition, the treatment is sustained until a desired suppression of disease symptoms occurs. Thus, one or more doses of about 20 mg/m², 85 mg/m², 90 mg/m², 125 mg/m², 200 mg/m², 400 mg/m², 500 mg/m², 600 mg/m² (or any combination thereof) may be administered to the patient. Such doses may be administered intermittently, e.g., every week or every two, three weeks, four, five, or six (e.g., such that the patient receives from about two to about twenty, e.g., about six doses of the additional agent). An initial higher loading dose, followed by one or more lower doses may be administered. However, other dosage regimens may be useful. The progress of this therapy is easily monitored by conventional techniques and assays.

In one embodiment, the subject has never been previously administered any drug(s) to treat cancer. In another embodiment, the subject or patient have been previously administered one or more medicaments(s) to treat cancer. In a further embodiment, the subject or patient was not responsive to one or more of the medicaments that had been previously administered. Such drugs to which the subject may be non-responsive include, for example, anti-neoplastic agents, chemotherapeutic agents, cytotoxic agents, and/or growth inhibitory agents.

### VI. Diagnostic Kits and Compositions

Provided herein are diagnostic kits including one or more reagents (e.g., polypeptides or polynucleotides) for determining the presence of a biomarker (e.g., PHLDA1, SPRY2, SPRY4, DUSP4, DUSP6, CCND1, EPHA2, EPHA4, ETV4, and ETV5) in a sample from an individual or patient with a disease or disorder (e.g., a proliferative cell disorder (e.g., cancer (e.g., lung cancer, breast cancer, skin cancer, colorectal cancer, stomach cancer, lymphoid cancer, ovarian cancer, and cervical cancer))). In some instances, the presence of the biomarker in the sample identifies a patient with a higher likelihood of benefiting from treatment with a MAPK signaling inhibitor. In some instances, the presence of the biomarker in the sample indicates a higher likelihood of efficacy when the individual is treated with a MAPK signaling inhibitor. In some instances, the absence of the biomarker in the sample indicates a lower likelihood of efficacy when the individual with the disease is treated with the MAPK signaling inhibitor. Optionally, the kit may further include instructions to use the kit to identify a patient with a higher likelihood of benefiting from treatment with a MAPK signaling inhibitor. In another instance, the kit may further include instructions to use the kit to select a medicament (e.g., a medicament including a MAPK signaling inhibitor, such as a MEK inhibitor, an ERK inhibitor, a BRAF inhibitor, a CRAF inhibitor, a RAF inhibitor, or combinations thereof) for treating the disease or disorder (e.g., cancer) if the individual expresses the biomarker (e.g., expresses the biomarker at an increased level) in the sample.

Provided herein are also compositions including polypeptides or polynucleotides capable of determining the expression level of at least one or more genes selected from the group consisting of DUSP6, ETV4, SPRY2, SPRY4, PHLDA1, ETV5, DUSP4, CCND1, EPHA2, and EPHA4 to be used according to any method of the invention. In some instances, the composition includes polypeptides capable of determining the expression level of at least four genes selected from the group consisting of DUSP6, ETV4, SPRY2, SPRY4, PHLDA1, ETV5, DUSP4, CCND1, EPHA2, and EPHA4. In other instances, the composition includes polynucleotides capable of determining the expression level of at least four genes selected from the group consisting of DUSP6, ETV4, SPRY2, SPRY4, PHLDA1, ETV5, DUSP4, CCND1, EPHA2, and EPHA4. In other instances, the composition includes polypeptides and polynucleotides capable of determining the expression level of at least four genes selected from the group consisting of DUSP6, ETV4, SPRY2, SPRY4, PHLDA1, ETV5, DUSP4, CCND1, EPHA2, and EPHA4. In some instances, the composition is capable of determining the expression levels of DUSP6, ETV4, SPRY2, and SPRY4. In yet other instances, the composition includes polypeptides and/or polynucleotides capable of determining the expression level of at least a fifth, a sixth, a seventh, an eight, a ninth, or a tenth gene selected from the group consisting of DUSP6, ETV4, SPRY2, SPRY4, PHLDA1, ETV5, DUSP4, CCND1, EPHA2, and EPHA4. In other instances, the composition is capable of determining the expression levels of DUSP6, ETV4, SPRY2, SPRY4, and PHLDA1. In other instances, the composition is capable of determining the expression levels of DUSP6, ETV4, SPRY2, SPRY4, PHLDA1, and ETV5. In other instances, the composition is capable of determining the expression levels of DUSP6, ETV4, SPRY2, SPRY4, PHLDA1, ETV5, and DUSP4. In other instances, the composition is capable of determining the expression levels of DUSP6, ETV4, SPRY2, SPRY4, PHLDA1, ETV5, DUSP4, and CCND1. In other instances, the composition is capable of determining the expression levels of DUSP6, ETV4, SPRY2, SPRY4, PHLDA1, ETV5, DUSP4, CCND1, and EPHA2. In other instances, the composition is capable of determining the expression levels of DUSP6, ETV4, SPRY2, SPRY4, PHLDA1, ETV5, DUSP4, CCND1, EPHA2, and EPHA4.

### EXAMPLES

The following examples are provided to illustrate, but not to limit the presently claimed invention.

### Example 1: An Elastic-Net Regression Model to Predict MAPK Signaling Ihibitor Sensitivity

An elastic-net regression model (e.g., an elastic-net model) was used to accurately predict a patient's MAPK signaling inhibitor (e.g., MEK inhibitor) sensitivity (Barretina et al. Nature. 483:603-607, 2012). Cell viability data from cobimetinib (COTELLIC®) or trametinib treated cells and concomitant gene expression data (e.g., RNA-Seq expression data) were collected for 26,255 genes from 46 colon, 106 lung, and 37 pancreatic cell lines. The expression data (e.g., gene expression feature data) and viability data were used to derive an elastic-net model with an alpha=0.5 and an optimal lambda chosen by 5-fold cross-validation (Barretina et al. Nature. 483:603-607, 2012).

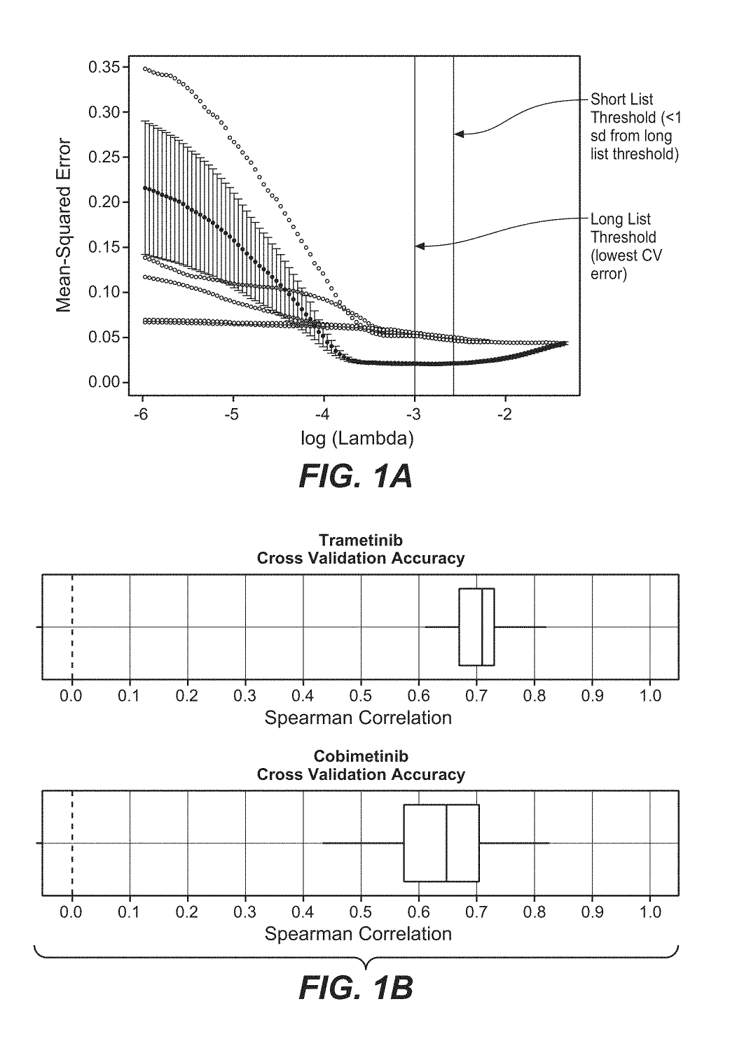

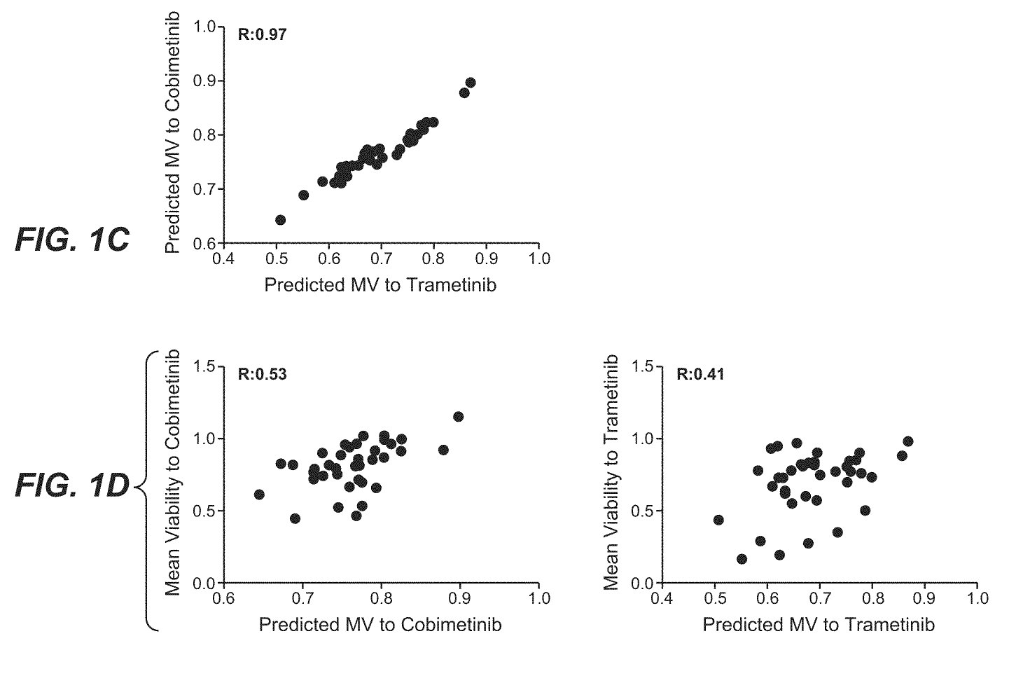

From the elastic-net model, two distinct predictive gene lists were established: (1) a list of genes corresponding to the lowest cross-validation error (long list) and (2) the shortest list of genes for which the cross validation error was still within one standard deviation of the lowest error (short list) (FIG. 1A). Analysis of the elastic-net model demonstrated that the model had a high cross-validation accuracy with predicted mean viabilities of the cell lines used to create the elastic-net model with experimentally derived mean viabilities to treatment with both trametinib and cobimetinib (FIGS. 1B and 1C). The predicted mean viabilities of the cell lines used to train the elastic-net model to treatment with trametinib (FIG. 1D, right) and with cobimetinib (FIG. 1D, left) correlated with the experimentally derived mean viabilities from 40 previously unscreened NSCLC cell lines (e.g., cell lines not used to derive the elastic-net model).

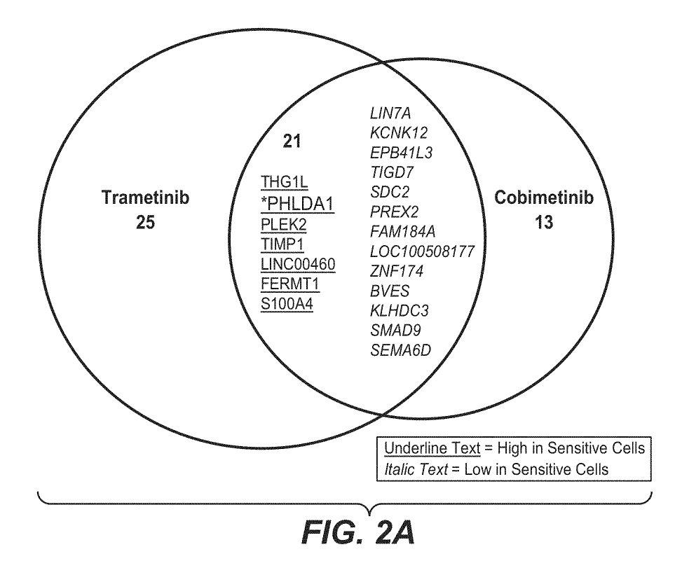

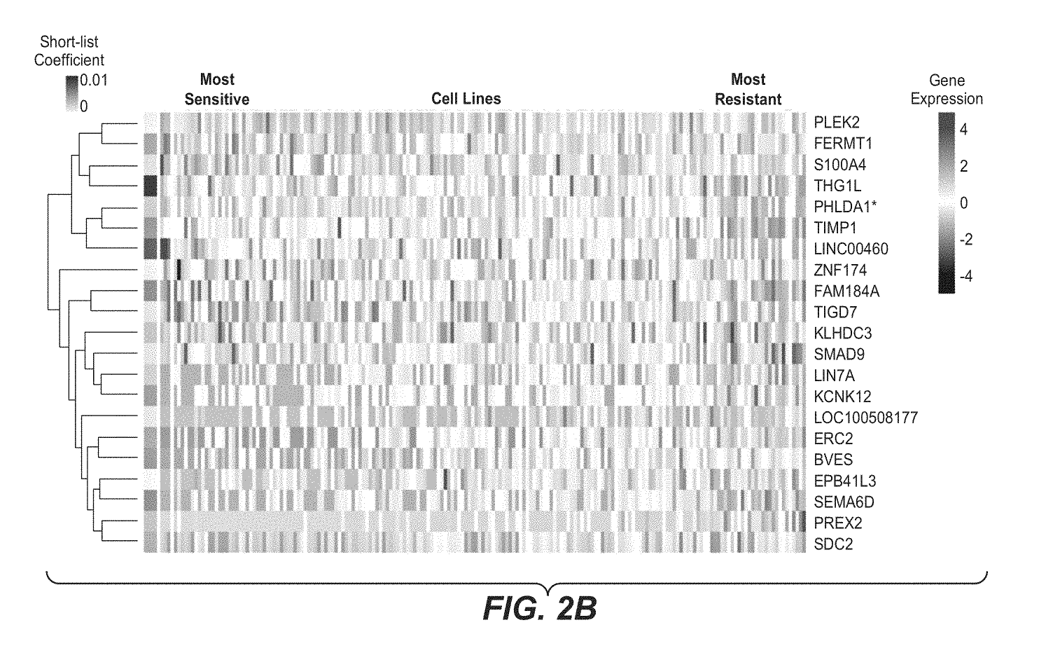

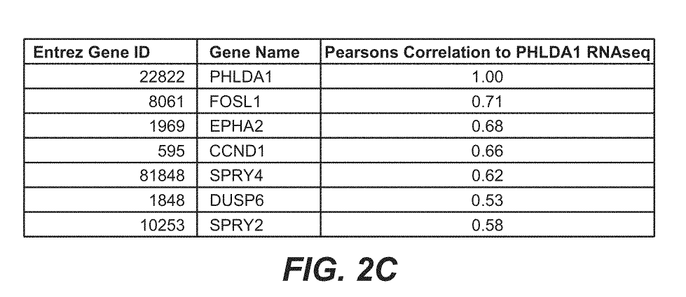

Multiple gene feature sets were found to be associated with MAPK signaling inhibitor sensitivity. Twenty-one gene feature sets, including a PHLDA1 gene set (FIG. 2C), that were present on the short list were associated with sensitivity to either trametinib, cobimetinib, or both drugs (FIG. 2A). The elastic-net model groups each gene with other similarly correlated genes based on expression level and mean cell viability (e.g., MAPK signaling inhibitor sensitivity versus resistance) to form a gene feature set (e.g., the PHLDA1 gene feature set as shown in FIG. 2B). The seven gene feature sets in FIG. 2A (left column) contain genes whose expression is correlated with high MEK inhibitor sensitivity. The fourteen gene feature sets in FIG. 2A (right column) contain gene feature sets whose expression is inversely correlated with MEK inhibitor sensitivity.

### Example 2: A MAPK Activity Score for Predicting MAPK Signaling Inhibitor Sensitivity

The PHLDA1 gene feature set identified by the elastic-net model contained a number of MAPK-specific genes associated with MAPK signaling (FIG. 2C). In order to use the expression of these MAPK-specific genes as a predictive biomarker, the gene feature set was first expanded to include additional MAPK-specific genes (e.g., DUSP4, EPHA4, ETV4, and ETV5). From the expression data of the MAPK-specific gene feature set (i.e., PHLDA1, SPRY2, SPRY4, DUSP4, DUSP6, CCND1, EPHA2, EPHA4, ETV4, and ETV5) an aggregated MAPK activity score, reflective of the level of MAPK signaling within a sample, was calculated according to the algorithm:

$$\frac{Σ {z}_{i}}{\sqrt{n}} ,$$

where zᵢ is the z-score of each gene reads per kilobase per million (RPKM), normalized across all samples, or to a set of housekeeping genes, and n is the number of genes comprising the set.

NSCLC GEM Model

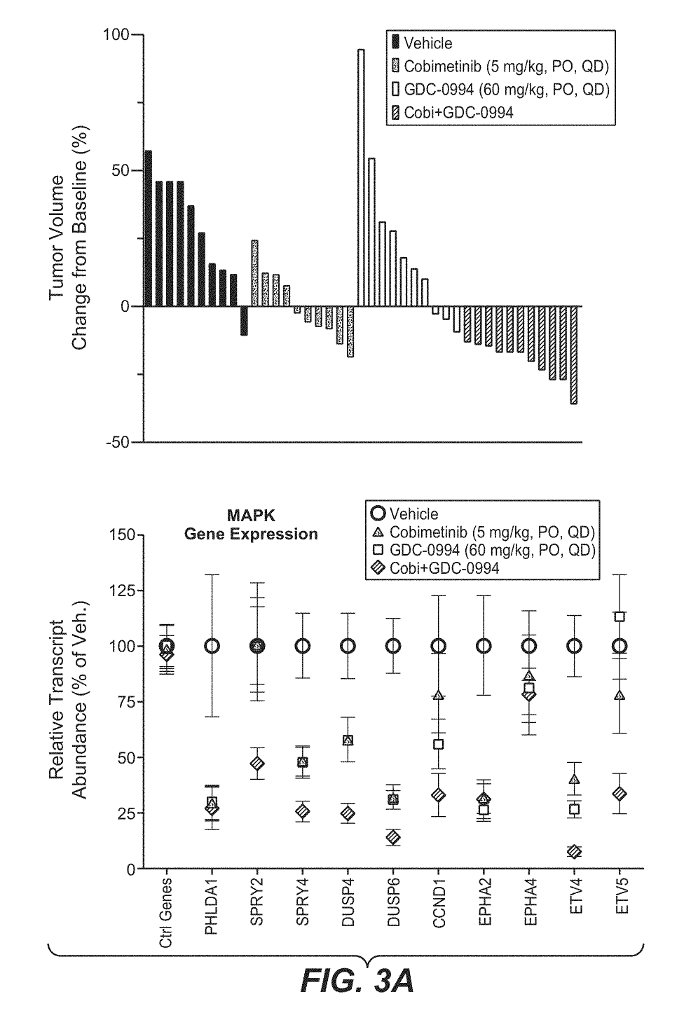

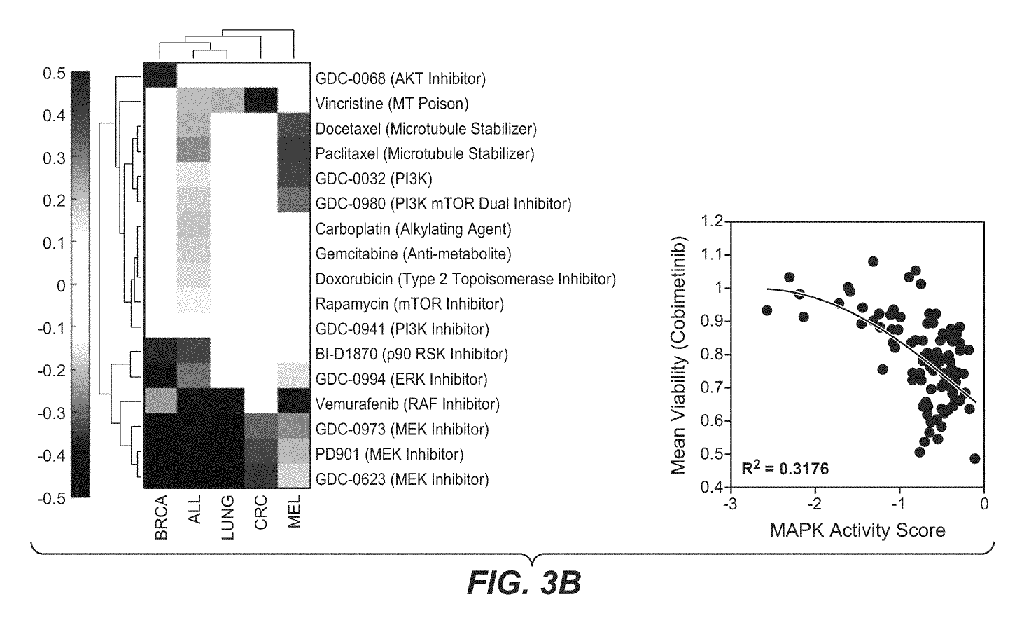

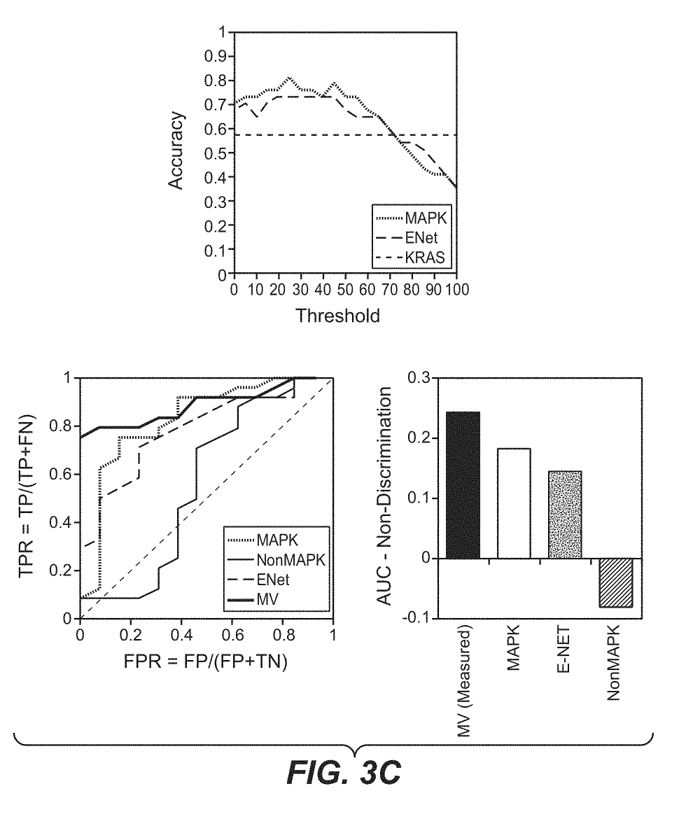

The set of ten robust MAPK-responsive genes (e.g., PHLDA1, SPRY2, SPRY4, DUSP4, DUSP6, CCND1, EPHA2, EPHA4, ETV4, and ETV5) was used to predict MEK inhibitor sensitivity (FIG. 3A) in an NSCLC GEM mouse model (LSL-KrasG12D/+, P53FRT/FRT-Adeno-CRE in C57B15 mice). NSCLC GEM mice were treated with 5 mg/kg cobimetinib, 60 mg/kg GDC-0994, or a combination of both administered orally once per day for 14 days. Tumor volume changes, measured by micro-computed tomography, and RNA from tumor samples were collected six-hour post-last dose following three days of treatment. The RNA was analyzed by Nanostring to measure MAPK gene expression. The degree of modulation (e.g., reduction) of MAPK gene expression after treatment with a MAPK signaling inhibitor correlated with tumor growth response (e.g., a change in tumor volume (e.g., a reduction in the size of a tumor)) (FIG. 3A).

Correlation of MAPK Activity Score and the Mean Viability of Cell Lines in Response to Drug Treatment

Gene expression data (e.g., RNA-Seq expression data) from the set of ten MAPK-specific genes (e.g., PHLDA1, SPRY2, SPRY4, DUSP4, DUSP6, CCND1, EPHA2, EPHA4, ETV4, and ETV5) were used to calculate a MAPK activity score, as described above, for >1000 cell lines, classified by tissue type and mutational status (e.g., BRAF-mut, RAS-mut (HRAS, KRAS, NRAS), and RAF/RAS wild-type (WT)) across multiple indications, including lung, breast (BRCA), CRC (colorectal), and melanoma. The MAPK activity score was found to correlate to sensitivity (e.g., mean viability) (FIG. 3B). Spearman rank correlation coefficients were also calculated from the MAPK activity score and mean viability data (e.g., sensitivity to treatment 95 tested drugs) for each indication (e.g., lung, breast (BRCA), CRC (colorectal), and melanoma) and across all cell lines (Pan-Cancer) (FIG. 3B, right panel). Correlations were similarly calculated for each of the 10 genes comprising the MAPK gene set.

Accuracy Comparison of Each of the Predictors

Accuracy and false positive (FP)/false negative (FN) rates were evaluated in a comparison of the elastic-net model, the MAPK activity score, and the KRAS mutation status ability to determine MAPK signaling inhibitor sensitivity (FIG. 3C). To assess how well each predictor (e.g., elastic net model, MAPK activity score, and KRAS status) classified the 40 NSCLC cell lines as cobimetinib-sensitive (IC₅₀<1 μM) versus resistant (IC₅₀>1 μM), the threshold for calling sensitive versus resistant was varied from 0-100% biomarker positive cells, over 5% intervals. The MAPK activity score was found to be more accurate than the elastic-net model in predicting MAPK cobimetinib sensitivity (FIG. 3C, top). Receiver operating characteristic (ROC) curves were generated by similarly varying the threshold for calling sensitive versus resistant cell lines and calculating FP and FN rates at each point for each predictor (FIG. 3C, bottom left). As a negative control, an activity score computed from four non-MAPK genes was also included in the comparison. The ROC curve data are summarized as area under the curve (AUC) by subtracting the zero predictive value line from the data (FIG. 3C, bottom right).

### Example 3: MAPK Activity Score and MAPK Inhibitor Sensitivity across Multiple Cancer Types

NSCLC Cell Line Validation Set

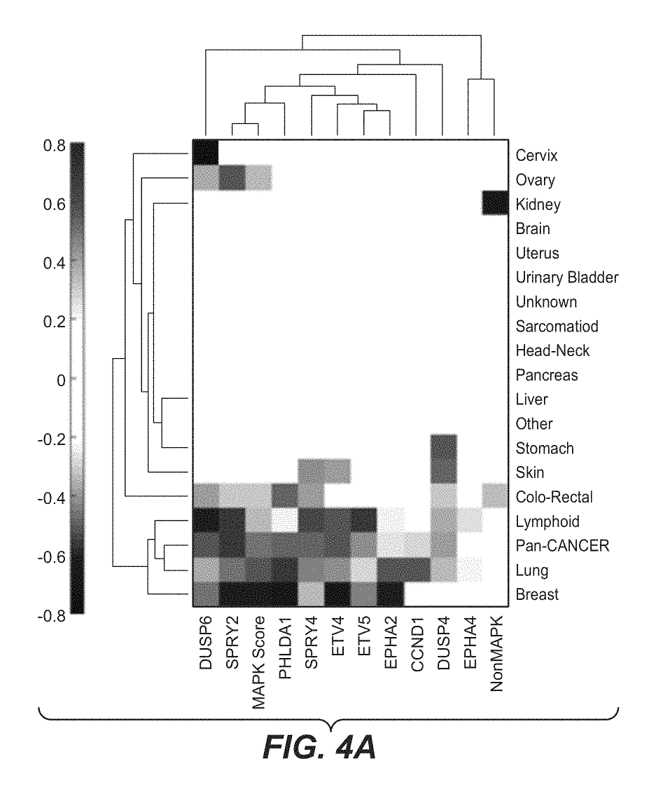

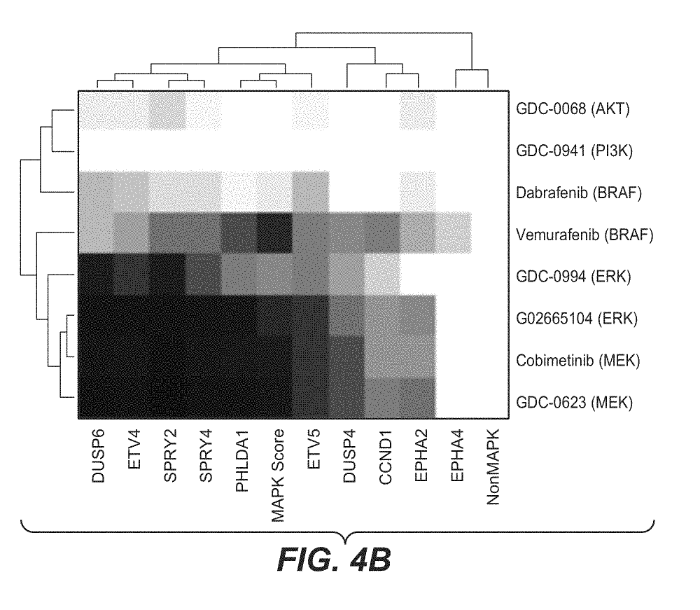

Forty NSCLC cell lines that had not been used in training the elastic-net model were tested for sensitivity to cobimetinib and trametinib. Cells were seeded at 5000 cells/well and treated with 0-10 mM of each drug (e.g., cobimetinib and trametinib) for 72 hours. Cell viability was measured using CellTiter-Glo. Mean viabilities were calculated across the dose range for each cell line. Correlation of gene expression data (RNA-Seq) from each individual MAPK-specific gene (i.e., PHLDA1, SPRY2, SPRY4, DUSP4, DUSP6, CCND1, EPHA2, EPHA4, ETV4, and ETV5) that makes up the MAPK activity score to sensitivity (e.g., mean viability) of >1000 cell lines to cobimetinib across multiple indications demonstrated that individual MAPK gene expression may predict MEK sensitivity and inversely correlate with sensitivity to MAPK inhibition (FIG. 4A). Expression of the individual MAPK genes that make up the MAPK activity score correlate with sensitivity to multiple MAPK pathway inhibitors, but not PI3K/AKT inhibitors (FIG. 4B).

Correlation of MAPK Activity Scores Dervived from Tumors and Mean Viability of Cell Lines to MAPK Inhibition

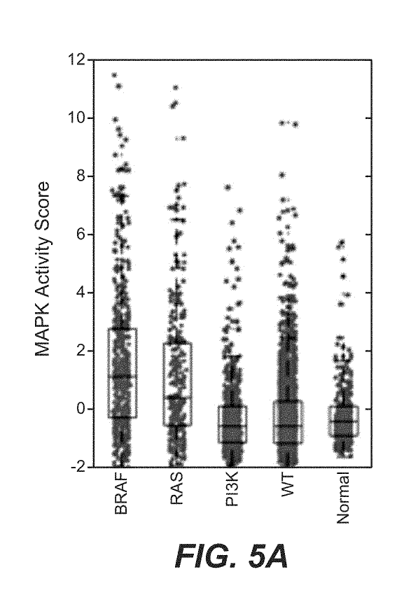

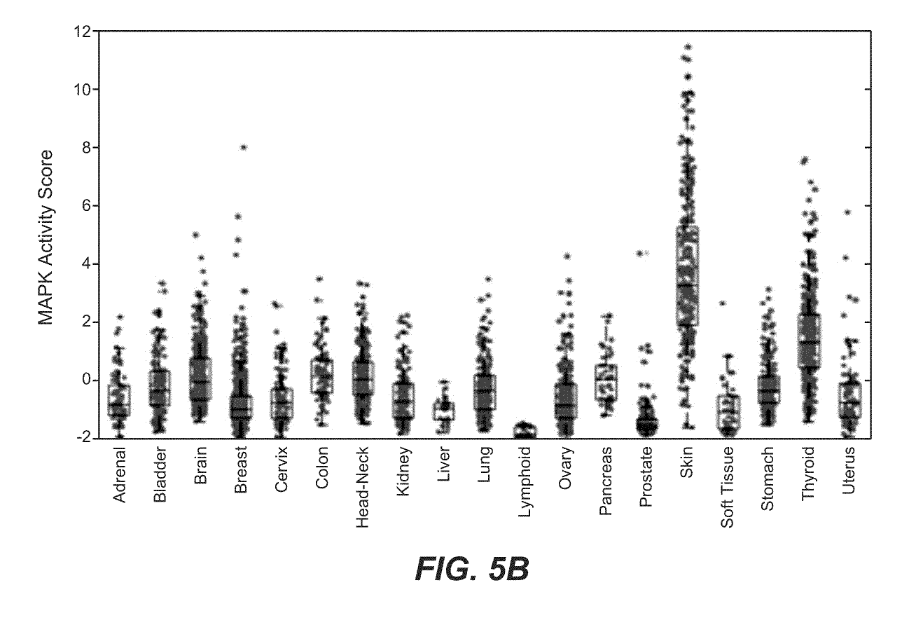

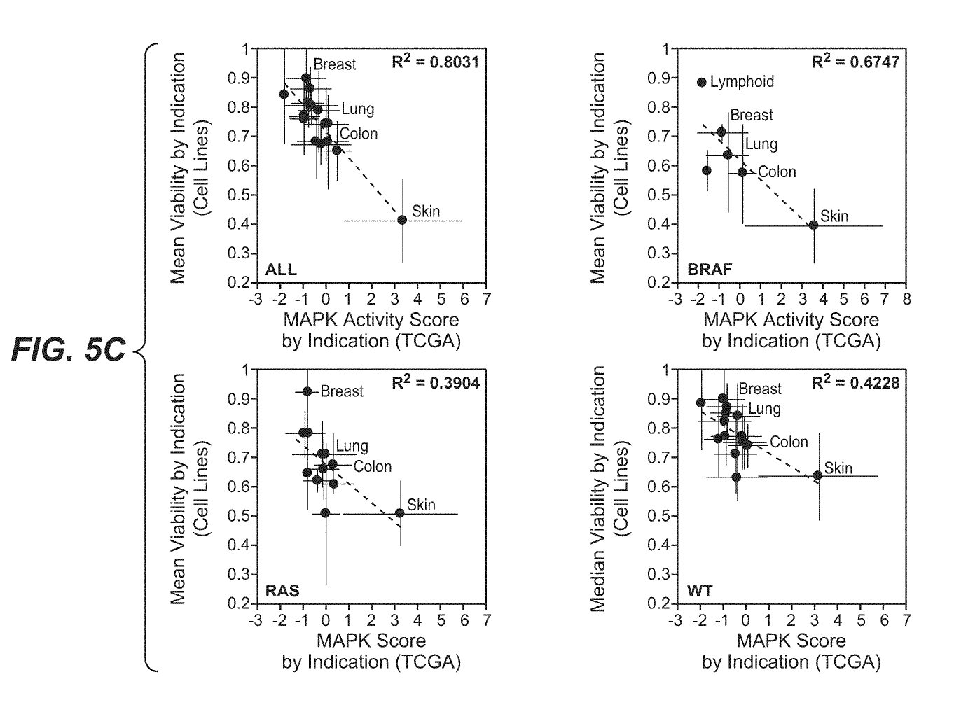

MAPK activity scores were computed for all tumor samples across different indications represented in The Cancer Genome Atlas (TCGA), classified by mutational status (e.g., BRAF-mutant, RAS-mutant and Wild-type) (FIGS. 5A and 5B). The MAPK activity score was highest in tumors, which are known to have the highest dependence on MAPK signaling due to mutation status (e.g., BRAF mutants) (FIG. 5A) and due to tissue source (e.g., skin) (FIG. 5B). Comparing clinical gene expression to cell line drug sensitivity to cobimetinib, the average MAPK activity score for each tissue type as measured in TCGA was correlated to the average mean viability for cell lines of the same tissue type for all samples (FIG. 5C, top left), BRAF-mutant samples (FIG. 5C, top right), RAS-mutant samples (FIG. 5C, bottom left), and wild-type samples (FIG. 5C, bottom right).

### Example 4: Clinical Validation of the MAPK Activity Score for Predicting MAPK Signaling Inhibitor Sensitivity

Study Design and Treatment

The coBRIM Trial is a multicenter, randomized, double-blind, placebo-controlled phase III study to evaluate the safety and efficacy of vemurafenib alone (e.g., a BRAF inhibitor alone) and vemurafenib in combination with cobimetinib (GDC-0973) (e.g., a BRAF inhibitor in combination with a MEK inhibitor), a mitogen-activated protein kinase (MEK) inhibitor (e.g., a MAPK signaling inhibitor), in previously untreated BRAF V600 mutation-positive patients with unresectable locally advanced or metastatic melanoma. Patients were randomized to one of two treatment arms, Arm A: vemurafenib 960 mg twice a day (days 1-28 of each cycle) and placebo (days 1-21 of each cycle); Arm B: vemurafenib 960 mg twice a day (days 1-28 of each cycle) and cobimetinib (GDC-0973) 60 mg once daily (days 1-21 of each cycle). Patients received treatment, supplied as tablets, until disease progression, unacceptable toxicity, or withdrawal of consent.

The primary outcomes for this study was progression-free survival, defined as the time from randomization to the first occurrence of disease progression, as determined by the investigator using Response Evaluation Criteria in Solid Tumors v1.1, or death from any cause, whichever came first. Disease progression was defined as: (1) at least a 20% increase in the sum (the increase in the sum must be at least 5 mm) of diameters of target lesions, taking as reference the smallest sum during the study; (2) unequivocal progression of existing non-target lesions; or (3) the appearance of 1 or more new lesions. Secondary outcomes for this study were overall survival, percentage of participants with an objective response, and duration of response assessed by Response Evaluation Criteria in Solid Tumors v1.1 and safety.

Prognostic biomarker MAPK activity scores were computed for each patient enrolled in the vemurafenib arm of the coBRIM phase III clinical trial in melanoma. Kaplan-Meier curves for progression-free survival (e.g., PFS) were plotted using the median value of the MAPK activity score to classify patients as MAPK-high or MAPK-low. Cox-proportional hazard regression models were then used to fit each treatment arm separately, using MAPK-high and MAPK-low, with or without further classification according to Cell Cycle or Immune baseline gene expression signatures, as independent predictors of PFS to calculate the hazard ratio and associated p-values.

Patients

Patients were eligible for enrollment in the study if they were 18 years and older with histologically confirmed melanoma, either unresectable stage IIIc or stage IV metastatic melanoma, as defined by the American Joint Committee on Cancer 7th edition. The unresectability of stage IIIc disease was confirmed by a surgical oncologist. Eligible patients were naïve to treatment for locally advanced unresectable or metastatic disease (e.g., no prior systemic anti-cancer therapy for advanced disease; stage IIIc and IV), however prior adjuvant immunotherapy (including ipilimumab) was allowed. Eligible patients could provide documentation of BRAF V600 mutation-positive status in melanoma tumor tissue (archival or newly obtained tumor samples) using the cobas 4800 BRAF V600 mutation test. Eligible patient had measurable disease per Response Evaluation Criteria in Solid Tumors (RECIST) v1.1 and an Eastern Clinical Oncology Group performance status of 0 or 1. Eligible patients provided consent to provide archival for biomarker analyses and to undergo tumor biopsies for biomarker analyses. Eligible patient had a life expectancy 12 weeks and adequate hematologic and end organ function.

Patient exclusionary criteria included a history of prior rapidly accelerated fibrosarcoma (e.g., RAF) or mitogen-activated protein kinase pathway inhibitor treatment, palliative radiotherapy within 14 days prior to the first dose of study treatment, major surgery or traumatic injury within 14 days prior to first dose of study treatment, or an active malignancy other than melanoma that could potentially interfere with the interpretation of efficacy measures. Patients who had a previous malignancy within the past 3 years were excluded except for patients with resected basal cell carcinoma (BCC) or squamous cell carcinoma (SCC) of the skin, melanoma in-situ, carcinoma in-situ of the cervix, and carcinoma in-situ of the breast. Patient with a history of or evidence of retinal pathology on ophthalmological examination that is considered a risk factor for neurosensory retinal detachment, retinal vein occlusion (RVO), or neovascular macular degeneration were excluded. Patients with uncontrolled glaucoma with intraocular pressure, serum cholesterol Grade 2, hypertriglyceridemia Grade 2, hyperglycemia (fasting) Grade 2, a history of clinically significant cardiac dysfunction. Patients with active central nervous system (CNS) lesions (including carcinomatous meningitis) were excluded. However, patients were eligible if all known CNS lesions have been treated with stereotactic therapy or surgery, and there has been no evidence of clinical and radiographic disease progression in the CNS for 3 weeks after radiotherapy or surgery.

Results

A total of 495 patients (Table 1) were enrolled in the study beginning in January 2013, with a total of 247 patients in Arm A (e.g., placebo+vemurafenib) and 246 patients in Arm B (e.g., cobimetinib+vemurafenib) being treated. At the time of the data cutoff (i.e., May 9, 2014; study still ongoing) a total of 181 patients in Arm A (e.g., placebo+vemurafenib) and 199 patients in Arm B (e.g., cobimetinib+vemurafenib) had completed the study. Table 2 summarizes the baseline characteristics of the patients. For all evaluable patients the median progression free survival in Arm A (e.g., placebo+vemurafenib; 248 participants analyzed) was 6.21 (5.55 to7.39) months and 9.89 in Arm B (e.g., cobimetinib+vemurafenib; 247 participants analyzed). The percentage of patients with an objective response rate (ORR) in Arm A (e.g., placebo+vemurafenib; 248 participants analyzed) was 44.8 (38.46 to 51.18) percent and 67.39 (61.39 to 73.41) percent in Arm B (e.g., cobimetinib+vemurafenib; 247 participants analyzed). As the study is ongoing, overall survival and the duration of response has not yet been determined.

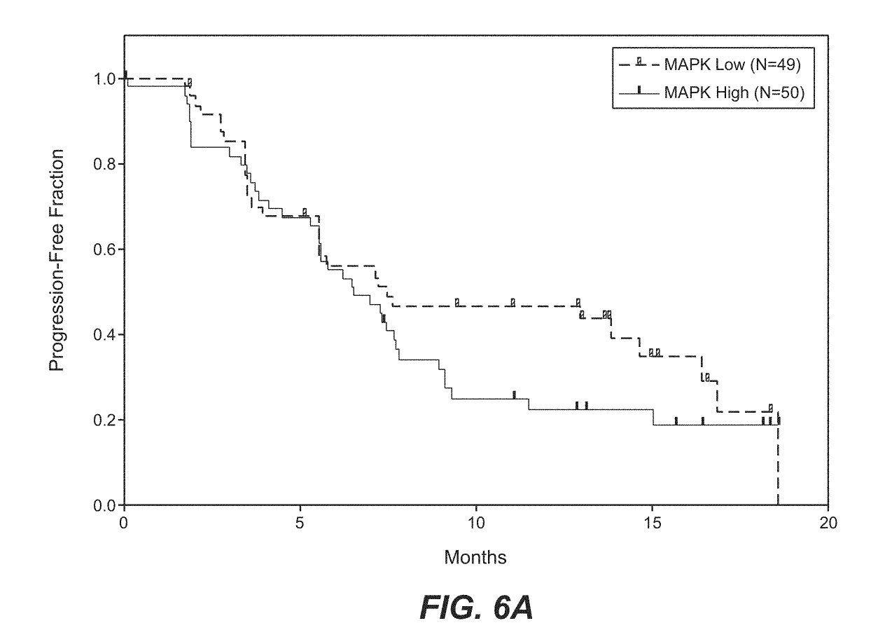

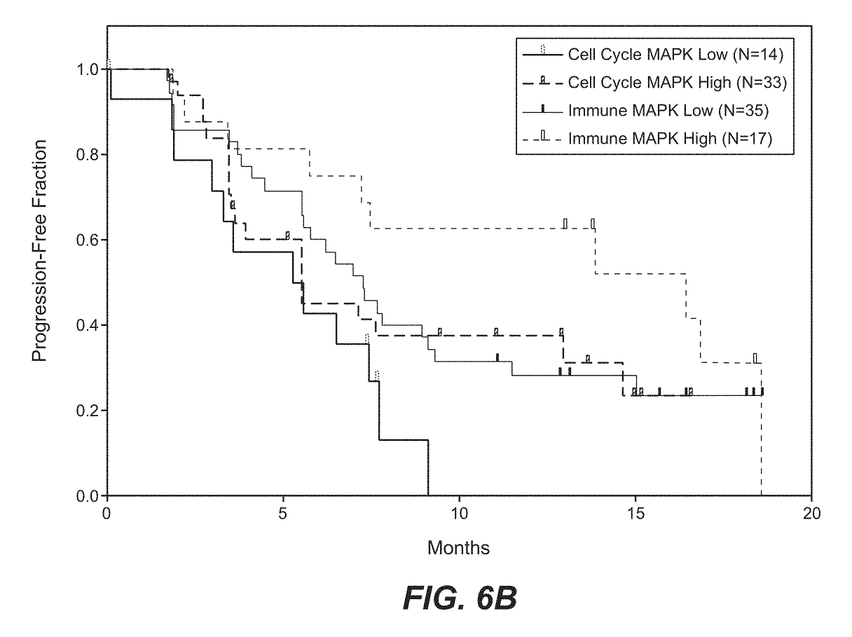

Patients with a high MAPK activity score trend towards a longer PFS to vemurafenib than those with a low MAPK activity score (FIG. 6A). Further classification of these samples according to previously characterized baseline gene expression signatures, Cell Cycle (highly proliferative tumors with a low immune infiltrate) and Immune (higher immune infiltrate and lower proliferation), shows that patients in the Cell Cycle/high MAPK activity score group do better than those in the Cell Cycle/low MAPK activity score group (FIG. 6B).

TABLE 1
Overall Study

| | Arm A: Placebo + | Arm B: Cobimetinib + |
|---|---|---|
| | Vemurafenib | Vemurafenib |
| STARTED | 248 | 247 |
| Received Treatment | 247 | 246 |
| COMPLETED | 181 | 199 |
| NOT COMPLETED | 67 | 48 |
| Death | 51 | 34 |
| Lost to Follow-up | 3 | 1 |
| Withdrawal by Subject | 13 | 10 |
| Physician Decision | 0 | 3 |

TABLE 2
Baseline Characteristics of Participants

| | Placebo + | Cobimetinib + | |
|---|---|---|---|
| | Vemurafenib | Vemurafenib | Total |
| Number of Participants | 248 | 247 | 495 |
| Age (years) | 55.3 (13.8) | 54.9 (14.0) | 55.1 (13.9) |
| Mean (Standard Deviation) | | | |
| Female | 108 | 101 | 209 |
| Male | 140 | 146 | 286 |

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of identifying a patient having a cancer who may benefit from treatment comprising one or more MAPK (mitogen-activated protein kinase) signaling inhibitors, the method comprising determining an expression level of at least one gene selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level identifies the patient as one who may benefit from treatment comprising one or more MAPK signaling inhibitors.

2. A method of optimizing therapeutic efficacy for treatment of a patient having a cancer, the method comprising determining an expression level of at least one gene selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment comprising one or more MAPK signaling inhibitors.

3. A method of predicting responsiveness of a patient having a cancer to treatment comprising one or more MAPK signaling inhibitors, the method comprising determining an expression level of at least one gene selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment comprising one or more MAPK signaling inhibitors.

4. A method of selecting a treatment for a patient having a cancer, the method comprising determining an expression level of at least one gene selected from the group consisting of DUSP6, ETV4, SPRY2, SPRY4, PHLDA1, ETV5, DUSP4, CCND1, EPHA2, and EPHA4 in a sample obtained from the patient, wherein an increased expression level of the at least one gene in the sample as compared to a reference level indicates that the patient has an increased likelihood of benefiting from treatment comprising one or more MAPK signaling inhibitors.

5. The method of any one of claims 1-4, wherein the method comprises determining the expression levels of at least four genes selected from DUSP6, ETV4, SPRY2, SPRY4, PHLDA1, ETV5, DUSP4, CCND1, EPHA2, and EPHA4.

6. The method of claim 5, wherein the at least four genes comprise DUSP6, ETV4, SPRY2, and SPRY4.

7. The method of claim 5 or 6, wherein the method comprises determining the expression levels of at least five genes selected from DUSP6, ETV4, SPRY2, SPRY4, PHLDA1, ETV5, DUSP4, CCND1, EPHA2, and EPHA4.

8. The method of claim 7, wherein the at least five genes comprise DUSP6, ETV4, SPRY2, SPRY4, and PHLDA1.

9. The method of any one of claims 5-8, wherein the method comprises determining the expression levels of at least six genes selected from DUSP6, ETV4, SPRY2, SPRY4, PHLDA1, ETV5, DUSP4, CCND1, EPHA2, and EPHA4.

10. The method of claim 9, wherein the at least six genes comprise DUSP6, ETV4, SPRY2, SPRY4, PHLDA1, and ETV5.

11. The method of any one of claims 5-10, wherein the method comprises determining the expression levels of at least seven genes selected from DUSP6, ETV4, SPRY2, SPRY4, PHLDA1, ETV5, DUSP4, CCND1, EPHA2, and EPHA4.

12. The method of claim 11, wherein the at least seven genes comprise DUSP6, ETV4, SPRY2, SPRY4, PHLDA1, ETV5, and DUSP4.

13. The method of any one of claims 5-12, wherein the method comprises determining the expression levels of at least eight genes selected from DUSP6, ETV4, SPRY2, SPRY4, PHLDA1, ETV5, DUSP4, CCND1, EPHA2, and EPHA4.

14. The method of claim 13, wherein the at least eight genes comprise DUSP6, ETV4, SPRY2, SPRY4, PHLDA1, ETV5, DUSP4, and CCND1.

15. The method of any one of claims 5-14, wherein the method comprises determining the expression levels of at least nine genes selected from DUSP6, ETV4, SPRY2, SPRY4, PHLDA1, ETV5, DUSP4, CCND1, EPHA2, and EPHA4.

16. The method of claim 15, wherein the at least nine genes comprise DUSP6, ETV4, SPRY2, SPRY4, PHLDA1, ETV5, DUSP4, CCND1, and EPHA2.

17. The method of any one of claims 5-16, wherein the method comprises determining the expression levels of DUSP6, ETV4, SPRY2, SPRY4, PHLDA1, ETV5, DUSP4, CCND1, EPHA2, and EPHA4.

18. The method of any one of claims 1-17, further comprising determining a MAPK activity score, wherein the MAPK activity score is determined according to the algorithm:
$$\frac{Σ {z}_{i}}{\sqrt{n}} ,$$
where zᵢ is the z-score of each gene, normalized across all samples or to a set of housekeeping genes, and n is the number of genes comprising the set.

19. The method of claim 18, wherein a MAPK activity score greater than a median MAPK activity score is a high MAPK activity score and identifies a patient who has an increased likelihood of benefiting from treatment comprising one or more MAPK signaling inhibitors.

20. The method of claim 18, wherein a MAPK activity score less than a median MAPK activity score is a low MAPK activity score and identifies a patient who has an decreased likelihood of benefiting from treatment comprising one or more MAPK signaling inhibitors.

21. The method of any one of claims 1-20, wherein the patient has a high MAPK activity score and the method further comprises administering to the patient a therapeutically effective amount of one or more MAPK signaling inhibitors.

22. The method of claim 21, wherein the administering of the one or more MAPK signaling inhibitors is after the determining of the expression level of the at least one gene.

23. The method of claim 21, wherein the administering of the one or more MAPK signaling inhibitors is before the determining of the expression level of the at least one gene.

24. A method of treating a patient having a cancer, comprising administering to the patient a therapeutically effective amount of one or more MAPK signaling inhibitors, wherein the expression level of at least one gene selected from the group consisting of DUSP6, ETV4, SPRY2, SPRY4, PHLDA1, ETV5, DUSP4, CCND1, EPHA2, and EPHA4 in a sample obtained from the patient have been determined to be increased as compared to a reference level.

25. The method of claim 24, wherein the expression levels of at least four genes have been determined to be increased in the patient sample relative to a reference level.

26. The method of claim 25, wherein the expression levels of DUSP6, ETV4, SPRY2, and SPRY4 have been determined to be increased in the patient sample relative to a reference level.

27. The method of claim 25 or 26, wherein the expression levels of at least five genes have been determined to be increased in the patient sample relative to a reference level.

28. The method of claim 27, wherein the expression levels of DUSP6, ETV4, SPRY2, SPRY4, and PHLDA1 have been determined to be increased in the patient sample relative to a reference level.

29. The method of any one of claims 25-28, wherein the expression levels of at least six genes have been determined to be increased in the patient sample relative to a reference level.

30. The method of claim 29, wherein the expression levels of DUSP6, ETV4, SPRY2, SPRY4, PHLDA1, and ETV5 have been determined to be increased in the patient sample relative to a reference level.

31. The method of any one of claims 25-30, wherein the expression levels of at least seven genes have been determined to be increased in the patient sample relative to a reference level.

32. The method of claim 31, wherein the expression levels of DUSP6, ETV4, SPRY2, SPRY4, PHLDA1, ETV5, and DUSP4 have been determined to be increased in the patient sample relative to a reference level.

33. The method of any one of claims 25-32, wherein the expression levels of at least eight genes have been determined to be increased in the patient sample relative to a reference level.

34. The method of claim 33, wherein the expression levels of DUSP6, ETV4, SPRY2, SPRY4, PHLDA1, ETV5, DUSP4, and CCND1 are determined to be increased in the patient sample relative to a reference level.

35. The method of any one of claims 25-34, wherein the expression levels of at least nine genes have been determined to be increased in the patient sample relative to a reference level.

36. The method of claim 35, wherein the expression levels of DUSP6, ETV4, SPRY2, SPRY4, PHLDA1, ETV5, DUSP4, CCND1, and EPHA2 have been determined to be increased in the patient sample relative to a reference level.

37. The method of any one of claims 25-36, wherein the expression levels of DUSP6, ETV4, SPRY2, SPRY4, PHLDA1, ETV5, DUSP4, CCND1, EPHA2, and EPHA4 have been determined to be increased in the patient sample relative to a reference level.

38. The method of any one of claims 24-37, wherein a high MAPK activity score has been determined for the patient according to the algorithm:
$$\frac{Σ {z}_{i}}{\sqrt{n}} ,$$
where zᵢ is the z-score of each gene, normalized across all samples or to a set of housekeeping genes, and n is the number of genes comprising the set, wherein the high MAPK activity score is greater than the median MAPK activity score and identifies a patient who has an increased likelihood of benefiting from treatment comprising one or more MAPK signaling inhibitors.

39. The method of any one of claims 19-23 and 38, wherein the median MAPK activity score is a previously defined median MAPK activity score for the cancer.

40. The method of claim 39, wherein the previously defined median MAPK activity score is determined from a plurality of samples from patients having the cancer.

41. The method of any one of claims 1-40, wherein the sample obtained from the patient is a tissue sample, a whole blood sample, a plasma sample, or a serum sample.

42. The method of claim 41, wherein the tissue sample is a tumor tissue sample.

43. The method of any one of claims 1-42, wherein the expression level is an mRNA expression level.

44. The method of claim 43, wherein the mRNA expression level is determined by RNA-Seq, PCR, RT-PCR, gene expression profiling, serial analysis of gene expression, microarray analysis, or whole genome sequencing.

45. The method of claim 44, wherein the mRNA expression level is determined by RNA-Seq.

46. The method of any one of claims 1-42, wherein the expression level is a protein expression level.

47. The method of any one of claims 1-46, wherein the cancer is selected from the group consisting of a lung cancer, breast cancer, skin cancer, colorectal cancer, stomach cancer, lymphoid cancer, ovarian cancer, cervical cancer, peritoneal cancer, pancreatic cancer, glioblastoma, liver cancer, bladder cancer, colon cancer, rectal cancer, endometrial cancer, uterine cancer, salivary gland cancer, renal cancer, prostate cancer, vulval cancer, thyroid cancer, anal cancer, penile cancer, and head and neck cancer.

48. The method of any one of claims 1-47, wherein the cancer is selected from the group consisting of a lung cancer, breast cancer, skin cancer, colorectal cancer, stomach cancer, lymphoid cancer, ovarian cancer, and cervical cancer.

49. The method of claim 48, wherein the cancer is a lung cancer, breast cancer, skin cancer, colorectal cancer, or stomach cancer.

50. The method of claim 49, wherein the cancer is a lung cancer.

51. The method of claim 50, wherein the lung cancer is non-small cell lung cancer (NSCLC).

52. The method of claim 49, wherein the cancer is a skin cancer.

53. The method of claim 52, wherein the skin cancer is a melanoma.

54. The method of claim 53, wherein the melanoma is a metastatic melanoma.

55. The method of claim 53, wherein the melanoma is a locally advanced melanoma.

56. The method of any one of claims 1-55, wherein the one or more MAPK signaling inhibitors are selected from the group consisting of a MEK inhibitor, an ERK inhibitor, a BRAF inhibitor, a CRAF inhibitor, a RAF inhibitor, or combinations thereof.

57. The method of claim 56, wherein a MEK inhibitor is selected from the group consisting of cobimetinib, trametinib, binimetinib, selumetinib, pimasertinib, refametinib, GDC-0623, PD-0325901, and BI-847325, or a pharmaceutically acceptable salt thereof.

58. The method of claim 57, wherein the MEK inhibitor is cobimetinib or cobimetinib hemifumarate.

59. The method of claim 56, wherein the ERK inhibitor is ravoxertinib (GDC-0994), ulixertinib (BVD-523), or a pharmaceutically acceptable salt thereof.

60. The method of claim 59, wherein the ERK inhibitor is ravoxertinib or ravoxertinib besylate.

61. The method of claim 56, wherein the BRAF inhibitor is selected from the group consisting of vemurafenib, dabrafenib, encorafenib (LGX818), GDC-0879, XL281, ARQ736, PLX3603, RAF265, and sorafenib, or a pharmaceutically acceptable salt thereof.

62. The method of claim 61, wherein the BRAF inhibitor is vemurafenib.

63. The method of claim 56, wherein the MAPK signaling inhibitor is a CRAF inhibitor.

64. The method of claim 56, wherein the RAF inhibitor is a pan-RAF inhibitor.

65. The method of claim 64, wherein the pan-RAF inhibitor is selected from the group consisting of LY-3009120, HM95573, LXH-254, MLN2480, BeiGene-283, RXDX-105, BAL3833, regorafenib, and sorafenib, or a pharmaceutically acceptable salt thereof.

66. The method of any one of claims 1-65, further comprising administering to the patient an additional therapeutic agent.

67. The method of claim 66, wherein the additional therapeutic agent is an additional MAPK signaling inhibitor.

68. The method of claim 67, wherein the MAPK signaling inhibitors are co-administered.

69. The method of claim 67, wherein the MAPK signaling inhibitors are sequentially administered.

70. The method of any one of claims 67-69, wherein the method comprises administering cobimetinib and vemurafenib, or pharmaceutically acceptable salts thereof.

71. The method of claim 66, wherein the additional therapeutic agent is an anti-cancer agent.

72. The method of claim 71, wherein the anti-cancer agent and the one or more MAPK signaling inhibitors are co-administered.

73. The method of claim 71, wherein the anti-cancer agent and the one or more MAPK signaling inhibitors are sequentially administered.

74. The method of any one of claims 71-73, wherein the anti-cancer agent is selected from the group consisting of a chemotherapeutic agent, a growth inhibitory agent, a cytotoxic agent, an agent used in radiation therapy, an anti-angiogenesis agent, an apoptotic agent, an anti-tubulin agent, and an immunotherapy agent.

75. The method of claim 74, wherein the anti-cancer agent is a chemotherapeutic agent.

76. A kit for identifying a patient who may benefit from treatment comprising one or more MAPK signaling inhibitors, the kit comprising:
(a) polypeptides or polynucleotides capable of determining the expression level of the at least one gene selected from the group consisting of DUSP6, ETV4, SPRY2, SPRY4, PHLDA1, ETV5, DUSP4, CCND1, EPHA2, and EPHA4; and
(b) instructions for using the polypeptides or polynucleotides to identify a patient that may benefit from treatment comprising one or more MAPK signaling inhibitors.

77. A composition comprising polypeptides or polynucleotides capable of determining the expression level of at least four genes selected from the group consisting of DUSP6, ETV4, SPRY2, SPRY4, PHLDA1, ETV5, DUSP4, CCND1, EPHA2, and EPHA4.


SEQUENCE LISTING (20 sequences)

SEQ ID NO: 1 - RNA, 5913 nt, Homo sapiens
guggacgcagcgggcuuuggaaaggccccaaguuaaugaggcgugcgccggcugccgagcgccucuuggagcugggcuuucccccgcggugcgggcgccaggagccgccuuuuccgcugggugucacucggggguggggaagauggcccauucaaaagcgccgcgagggggcccggccagugcccuucagugagcgcucgcaagaggacggcagaggcccggcagcucggagcuccgggaccuuguggcgcaucaggacgcggcugucccucugccgggacccagagccgccgccgccgcucugccuccugcguguuagccuccucugcgcgcuccgggcaggcggccgugggagccgcuggggcgaggacggcgcgaggcugcugcugcugcccccggcccgcgcggcuggaaacggagaggccgagccaagcggcggccccucuuaugcugggaggaugcuggagaguagcggcugcaaagcgcugaaggagggcgugcuggagaagcgcagcgacggguuguugcagcucuggaagaaaaaguguugcauccucaccgaggaagggcugcugcuuaucccgcccaagcagcugcaacaccagcagcagcagcagcaacagcagcagcagcagcagcaacaacagcccgggcaggggccggccgagccgucccaacccaguggccccgcugucgccagccucgagccgccggucaagcucaaggaacugcacuucuccaacaugaagaccguggacuguguggagcgcaagggcaaguacauguacuucacuguggugauggcagagggcaaggagaucgacuuucggugcccgcaagaccagggcuggaacgccgagaucacgcugcagauggugcaguacaagaaucgucaggccauccuggcggucaaauccacgcggcagaagcagcagcaccugguccagcagcagccccccucgcagccgcagccgcagccgcagcuccagccccaaccccagccucagccucagccgcaaccccagccccaaucacaaccccagccucagccccaacccaagccucagccccagcagcuccacccguauccgcauccacauccacauccacacucucauccucacucgcacccacacccucacccgcacccgcauccgcaccaaauaccgcacccacacccacagccgcacucgcagccgcacgggcaccggcuucuccgcagcaccuccaacucugccugaaaggggcagcucccgggcaagacaagguuuugaggacuugaggaagugggacgagcacauuucuauugucuucacuuggaucaaaagcaaaacagucucuccgccccgcaccagaucaaguaguuuggacaucacccuacugaaaacuugcgauucuucuuaguuuucugcauacuuuucaucacgaugcaggaaacgauuucgagucaagaagacuuuuauuuaugaaccuuugaaaggaucgucuuguauggugaauuuucuaggagcgaugauguacuguaauuuuauuuuaauguauuuugauuuaugauuauuuauuaguuuuuuuuuaaaugcuuguucuaagacauuucugaauguagaccauuuuccaaaaaggaaacuuuauuuucaaaaaccuaauccguaguaauuccuaaucuuggagaauaaaaaagggcgguggaggggaaaacauuaagaauuuauucauuauuucucgaguacuuucagaaagucugacacuuucauuguugugccagcugguugaaauuaaaacucugauauuacuuuuuuugaggauuuuuauuuuuguuuuugcuuaaacauauaguuugucuagaaguuuaaaaagcuaaaaguuaaaaaugguguaauuaugaaaaucuaacacucaagauaguuucuaaaaggaaaucaguaguuaaggauaccugauuucaaaauauuuaaagcauaaccuaacugaugguaggaugauuguaucuugaauaugugguagggccacaucuauuguaggaaaaccuugcuuuuaucaucuguguguaaagggcuuaauaaggagaagaggccuuuugacugauuugugaguauaaaugcauuugcuguuucauuucaaaaauguuguggaggaaaagaguacauuuaacuuguauaagagaauauuuguacuccuguccaggcugcaggaccuuucuucgagagcuuugcacacuugacuugaaccacauuuucugaucccuuuacuuuguuuuagaagcacacugaaaaaucucguuguuuaaaguacaauuuguaaauauuucaaaggucuaggagucauaacuuuuguuuucauacugaaaaugauguugaucagagaaaccaacuguuuugcuuuucauugcucugugagaaauuugaggauucuguuuugcuguuagguaagcuaaacucagaaauugaaaaggaaaagacuggauaaacacaggauuuucaguaagaaaacaaccccagucuugucuuagaagccacuuguugaggagucuguugggggaaaaaagaggauaugcuuuuaaagguagaacaaaccuucuucuguguuaaaucaaaaggauguucaaaauccaccaggacagaugcuacuuggguuuaaauggagccauagaugauacaaaguccucuuggggcugaaaaucacuuccuauuugcauggcuuuacuaacugguuucuguuuuccauuaucuuuuucacagaaagucuuggucaguauuuuuccagcauuuaaauugaaacggucaguauuagaccacugcuagguuauguagucaagaaauaaaaauagaauuacaugcuacagaugucuuuauucuccuuccaucuagaaaggaguuccaaggucaaauuacuuuuuagugcaauaguuaaaugacauuuugagaucauaacucauauccaaaaaguugcagggaaaauuaaaauagcuuuccccuauuaagcuaauggcaaacaaaacuuaaguggacccccacuuccagugguuguuuagguugcaguugugaaaauaugcugccaacauuuaaaaacuuguuucauauguauauauguauacacauauaugaauauguauguauauauacauauaugagaacauguguguacacauauaugaauauguauauauguguauguauguauauauguauaugaaaugagagccacaucuaaagauuucuuaaaucaaguuugguucagcuuccuuagaacuguggcuguacuuuuugaggaguaccucauaguacuauauuuuuaaugcaugcaaaucauaauagcuccaaaugaaccacaguuuuuucccaauggaggauuuuuuuuuaauucuuguacuaaaaaaaaaaaauccauaccaaauauuuuuacaaauuaagauugauguagguuuuaaaaaaggcauuuguauguuguuagcuuacauauggggcuagguaauuucauugcuuaaaaagaugcgccuaggcucccucuugguggcuggauuucuuuuucuucgcccgugguggccaugguucuuaauagggccaccggaaucaugguuucuuuuuuuuuuuuuuuuuuugagauggagucucgcccugugacccaggcuggagugcaguggcacgaucucggcucacugcaaccucugccuccuggguucacgccauucuccugucucagccuccugaguagcugggacuacaggugaaugccaccacgcccggcugauuuuuguauuuuuaguagagaugggguuucaccauaguggucaggcuguucucgaacuccugaccucaggugauccaccugccuuggccucccaaagugcuaggauuacaggugugagccaccacacccggccccagaauaaugguuucuugacuuucuguagcccuuguuccuuagucugcugugauauuuauguugaccuuuaucauuuucuauucugaaccccucuuagcauuuaaugugaaaucuaagaaauuagaaguagaauggcuuuuauuguuuugacaccuuugaaauuauuauuaauaauuuuuccagagcaaaaaagcaaacacgcucaauaagacuaaacaaaacaaaauauaaauguacaucauuuaaugucccaguggcucuauucuaccuguaagaaaaugauacaaaaccaccuaagauauuuugaagccugacaaaucagcuucauggaaaaagguaaaaaaugcauuuuucaaccgaaagggcagauccaauagaagacccgcuccuuaaauaaacauaaaauguaaaaaguuggaaaauuaagaguaauguuccaucuggaaacugaacuuuuguccuugaacuuguguuggcaccaagccucauacacagugagcucaauaacuguugggacaaaggaaggaaggacaaaauguguaacuucccagcaucugggagaugcugucucuugccucacugaguguuccuuuucuuugcucucaugucauucccugagaacaaugaauucugggacaggcuaaacaucaugaugaaguuucuuaaacagacuuucuuaguggaaauccauuuagaucugggugugcucuauggggagugcugacgucaaagagcaaaugucuauaaggggcccuuuuaaaaugaacauuuuccucauugagcaagcugggauucucuaauguagaaaucaagccaucuuuauaauuucacuucagauguuuauguuuuuguuuuuuuugucuccaaugaugguaaaaauaaaaacuacgcauuacuuaaaggaguuucccucacauguaaacacuguuaggaagucuggauuaaguugaaaguccuguuuuaacuuuuuuucucucauauaccaaacacucuguauuucucuuaaagaagcccuuuaagagaaagcccuaauuuuauaucugacaguaaaguuugcugcaaguguaugaguucaaacacaucccuuguuuucugucccuaggggaaaagucauguaguuuuagcuuggcuccaguguuaauauuauauucaguagcagccuuagaagaguggucuaagacuugaaccuggagcaauuuuauagcacagaauccuacgaagauaggacugugaacauuuguuuucuuuuucgugugugucaaacuaacugguuuuugcuuuaccaauaaaauguccucggcagaguaaauuuuaaacgugaaaauuauagaucuugauauugaauccaucagugauucaagagauacaccuauuugccuaaaacaaccuaagauguauugguuauggaaucauguguuggauagguucuuaagaccuguuuccucaaaucuugacacaguuuucaaggguggcuuauugacuugcacgguugggcagauaauccagauuuaccuaagauuggguaaaaaagucaucugugacuuugcuggcagggcauuugcuaaguggaguacaggaucuaaaaggguuuucuuagaaagggcaauauuguccaaugaaguaagcagaaggacucuggguuagaagcaucugcacaaaaacuggugagaccuacucuccacugcucugcagcuggauggcugauggcaggcugagcaguggggaagcagguuuuaacaacagggaguccuuccaggucacuguauauugagaagaaacauaaaacuauugucuguuacauuccgaggucagccuucuucuuaacguuuuauaauaugcaaaugccagcuucuggaaagcaaguaucaucauguaccaaaugcuuuauacaccaucacauucaugaauuuuuuagcauggucagaacuuguguaaauaugucucuuagaugauuuuggggagaugugauuuauuuuucauauuuucaaaaugcauuucauuucaaauaaaguuaucuauugagacaaccga

SEQ ID NO: 2 - protein, 401 aa, Homo sapiens
MRRAPAAERLLELGFPPRCGRQEPPFPLGVTRGWGRWPIQKRREGARPVPFSERSQEDGRGPAARSSGTLWRIRTRLSLCRDPEPPPPLCLLRVSLLCALRAGGRGSRWGEDGARLLLLPPARAAGNGEAEPSGGPSYAGRMLESSGCKALKEGVLEKRSDGLLQLWKKKCCILTEEGLLLIPPKQLQHQQQQQQQQQQQQQQQPGQGPAEPSQPSGPAVASLEPPVKLKELHFSNMKTVDCVERKGKYMYFTVVMAEGKEIDFRCPQDQGWNAEITLQMVQYKNRQAILAVKSTRQKQQHLVQQQPPSQPQPQPQLQPQPQPQPQPQPQPQSQPQPQPQPKPQPQQLHPYPHPHPHPHSHPHSHPHPHPHPHPHQIPHPHPQPHSQPHGHRLLRSTSNSA

SEQ ID NO: 3 - RNA, 2032 nt, Homo sapiens
agggugagaaaaaggcagggugccggguggagggagugcccgccucugcuccguguggacagucuagcccggagcgggugggagcgucgcccugccuggcgccgggucucugcgguucggaagugcagcucucucucuuccguucuucguggucagccugggucacacuuauuuuauuuuuggaagcucgggcugacccagcgcaggaagaaagucaucaagcggucgcgaucauuuuccauuaccugaaagaguucagauguguucuaagccugcuggagugaccacacuuccaagaccugauggaggccagagcucagaguggcaacgggucgcagcccuugcugcagacgccccgugacgguggcagacagcguggggagcccgaccccagagacgcccucacccagcagguacaugucuugucucuggaucagaucagagccauccgaaacaccaaugaguacacagaggggccuacugucgucccaagaccugggcucaagccugcuccucgccccuccacucagcacaaacacgagagacuccacggucugccugagcaccgccagccuccuaggcuccagcacucgcagguccauucuucugcacgagccccucuguccagauccauaagcacggucagcucagggucgcggagcaguacgaggacaaguaccagcagcagcuccucugaacagagacugcuaggaucauccuucuccuccgggccuguugcugauggcauaauccgggugcaacccaaaucugagcucaagccaggugagcuuaagccacugagcaaggaagauuugggccugcacgccuacaggugugaggacuguggcaagugcaaauguaaggagugcaccuacccaaggccucugccaucagacuggaucugcgacaagcagugccuuugcucggcccagaacgugauugacuaugggacuuguguaugcugugugaaaggucucuucuaucacuguucuaaugaugaugaggacaacugugcugacaacccauguucuugcagccagucucacuguuguacacgauggucagccaugggugucaugucccucuuuuugccuuguuuaugguguuaccuuccagccaaggguugccuuaaauugugccagggguguuaugaccggguuaacaggccugguugccgcuguaaaaacucaaacacaguuugcugcaaaguucccacuguccccccuaggaacuuugaaaaaccaacauagcaucauuaaucaggaauauuacaguaaugaggauuuuuucuuucuuuuuuuaauacacauaugcaaccaacuaaacaguuauaaucuuggcacuguuaauagaaaggugggauagucuuugcuguuugcggugaaaugcuuuuuguccaugugccguuuuaacugauaugcuuguuagaacucagcuaauggagcucaaaguaugagauacagaacuuggugacccauguauugcauaagcuaaagcaacacagacacuccuaggcaaaguuuuuguuugugaauaguacuugcaaaacuuguaaauuagcagaugacuuuuuuccauuguuuucuccagagagaaugugcuauauuuuuguauauacaauaauauuugcaacugugaaaaacaaguugugccauacuacauggcacagacacaaaauauuauacuaauauguuguacauucggaagaaugugaaucaaucaguauguuuuuagauuguauuuugccuuacagaaagccuuuauuguaagacucugauuucccuuuggacuucauguauauuguacaguuacaguaaaauucaaccuuuauuuucuaauuuuuucaacauauuguuuaguguaaagaauauuuauuugaaguuuuauuauuuuauaaaaaagaauauuuauuuuaagaggcaucuuacaaauuuugccccuuuuaugaggaugugauaguugcugcaaaugagggguuacagaugcauauguccaauauaaaauagaaaauauauuaacguuugaaauuaaa

SEQ ID NO: 4 - protein, 315 aa, Homo sapiens
MEARAQSGNGSQPLLQTPRDGGRQRGEPDPRDALTQQVHVLSLDQIRAIRNTNEYTEGPTVVPRPGLKPAPRPSTQHKHERLHGLPEHRQPPRLQHSQVHSSARAPLSRSISTVSSGSRSSTRTSTSSSSSEQRLLGSSFSSGPVADGIIRVQPKSELKPGELKPLSKEDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 5 - RNA, 4941 nt, Homo sapiens
auucauaaaaaagccauuuucccaggcagugguugcaacaucgccgcggagguagcgagcugagcugacagcgcggagcuggcgcuguggagcgcagggagccuugccgguuccuccgaccggcgucugcgaguacagcggcggcuaaccugccccggcuucaggauuuacacagacguggggcgaugcuugugacccugcagcuccucaaaggccccuagaagccuguuucuccguacaguccaggaccuccagccccauggagcccccgaucccacagagcgcccccuugacucccaacucagucaugguccagccccuucuugacagccggaugucccacagccggcuccagcacccacucaccauccuacccauugaccaggugaagaccagccauguggagaaugacuacauagacaacccuagccuggcccugaccaccggcccaaagcggacccggggcggggccccagagcuggccccgacgcccgcccgcugugaccaggaugucacccaccauuggaucuccuucagcgggcgccccagcucugugagcagcagcagcagcacauccucugaccaacggcucuuagaccacauggcaccaccacccguggcugaccaggccucaccaagggcugugcgcauccagcccaaggugguccacugccagccgcuggaccucaagggcccggcggucccacccgagcuggacaagcacuucuugcugugcgaggccugugggaaguguaaaugcaaggagugugcauccccccggacguugccuuccugcugggucugcaaccaggagugccugugcucagcccagacucuggucaacuauggcacgugcauguguuuggugcagggcaucuucuaccacugcacgaaugaggacgaugagggcuccugcgcugaccaccccugcuccugcucccgcuccaacugcugcgcccgcugguccuucaugggugcucucuccguggugcugcccugccugcucugcuaccugccugccaccggcugcgugaagcuggcccagcguggcuacgaccgucugcgccgcccugguugccgcugcaagcacacgaacagcgucaucugcaaagcagccagcggggaugccaagaccagcaggcccgacaagccuuucugacaguuugugucgaagccccagugcucugccuggaaaccugguucucuucugacaucuaagaagacugcagcaaggucagagguuuuagccuccugaggcugaccuugcuagucugcccacucccuacccccagcuucggaaaauacagagaccaccaccacguacccuguauuccccaaggugaugaagaagcacuuuggggcuuuuuuucaggguccugaaacuuugugucaaacagacaaugcaggggcagggugugguuuggggggaaauuuuucuuuuucagaagacagaacacagauguggacacauauccggaaacugcagcugcuugaaugccuucccagccccuccuucucccucccucccuccgcccccccccccuuccucuuuuccauugucuuuggcucucacaggagcuagcugccugggaggaauuguuaacugaguaccaggguaccuuuaaagaagacccuuggagucuucuauaccuucuucuccuuccccaucucacuccaccccacuuugucccugaugucuuggggaagguguagaacacccuagcaguuccuauuguauauacuugggagccacugagaacagaggacggccagugaguccaagccucguuccuccuucugccuccccggagccacaggauggauuuaggagccacugcucagugcacuucucccuuccaacugcaucaacuaacucucggggguguucugcucaccacaccguccuucgguucuuacugagucacagacucgccugcccacuacguguccuggguucucucuacucagaucccuuccagaaacuuuauauggguagaggaagccagggcggcaaaugcgagaccaaauaucauuuugccaaugagucugaggcuguggucucuggauccagucauuauguuuuuauagaauaauuaaaccggaugcuaacgguguuuuaaaaaauaauaauaaaacaacuuguuuccuuuuggccacccccaggaagggcugauuucaaaaucugggggcgagcaaccucaaggaacacaauuucccucccuaucaacaagaggauuuuaacagcaaagaagagaggcagcaccucccauuggcagaaugaccgcugagccaggcuggguuuggguuucuucucuucugauucugcugcucacugucauagccuuuuguguauagugaugugucuguaucuuuaauguaaauagagagaugaugaaaaaagagucuauuuuaguguuaggaagccccagcaggggagucggaagagcuuggaagagcuggggagaggguaggggaaagguuuuuccaggggccacuggguuugagcccugcuucugugcacagccacaccacccucucccgacagcccucaaagacguagcaacucuuucucucaaggugcuaaaggacucagaaggugcagcacguccaguggguagguacuuguugcaugcaaaagcuguaguguaucugguccuuccuccccagcuuuugugugggguucuugcuuugugugguauuuuguuuuccccucuaaugagagggcauggccugagucagaagagcuaccccaggugaaacuggaagugcaugaggcagagcguccguagcauuuccaguuuguucuguauaggaacagaggugccuccgggaaggaggcagcgagguagguagcuaugauaggcaccuaaugcuucucaaggacuuauuuuuuccuucuugaagacuaguaguaacaucuuaugauuuagaguaaguugauuguaaccauagguauuuauugauuggaggaagggagggucauauuauuuucggcuuuauuuauguaacauuugcuagcuuguaaaaggcgaaugugaaauauugcaucugcauuuuccaaggcugauucguguagcuacccuugccacaguugugacggauguauggauguucuugaacauuucagaaggagugguagaaaaaaacacacauucagccaaccacuuauaugaauugaauguaucagaaguguacugaagggacuggagaugguuuuccucagaugagggggccccaaaauugauagugcacaucugcacgcuuucugcgaggccucagaacuucccagggccccucccucaaauugucuccaugggaaacuugacccaguggcaaguugcacuuuggugaucuugguggucuacacacccguucuguggagagucgauuuacauaagcuguguauacacacacacacacacacacacacaccccuaccccacacugacugucuaccgacaaagacccuauuuccuggcaaacggccuccugaacccugacuuuuuguguacauacuuguaaacacggauuuuucuggguuuugguuugcuuuuuccuuuuuucccccugccccuguucuagcuuguucuucuugguuugcuuucaaccugcuugauggaugucugcagagugcucucuaagaguccaccucagugccucgugugcucaguggucaugggaaaggagcgaaggaaccauccuugguucucccagcuugguuguguagcaaucccucagcauuguuuuucucagcuucuuggcaaaaauuaaaacaacaacaacaacaacaacaacaacaacaaacagaaggauaaacuggcuugccuguggacccuccccggcucuggggccagucgagagccacugagggacccagcacucagagacacaacacacauguguagcugcuucuggcugaguguguuuccugucaccaauggccuguuuggcuggacgaugccucggcuugaccuuuuuugaaaagugcugguuaguucccgccccugguaaaccugggguagguggggguucugucuuaacucgaggggcaccugggauccaggacgcuucuagggggcucuggcugcccguguuaaugaaggacagcgcuuccgcgagcacccugggaacugggucuuggguagcaaagcccucccagagaaaagauugggcacaacuaaggcuuuccugagcaggaagggggugaagaccaaucccuuccuuugguccuuugguacgcacccccucagagcugagauggaagacauggcuaguucuuuucagccuuguggagccugucagucgccaucauaccucgagugaggcccagcuagauaaugacuuguccaagauggcacacguggaaaguugaucugcaccagaacccggaugacugucaccuugaagcauccuguucuccuucugugcugucccaggaagugucuggcgggcgugggcagcacagcucuacacuguacgauucacuagggcauccugcgagccucacuagccuucugguucaugccuuugacaagcauuuuugugcccccucugcuuacugugacagucgaugaugaaucuugcguugccauuuucugcuguggguaacugcgugcagugucuugccuugcuuucucuucuuacugucccacagcuugguuucauguuacaaacagaaaagcucgaggcucccaccccgccacaucccaacuucauuucccccucacuguagcccauuuccaccccaccacaaaguugccacagguuuucuuuguauagaauauuuauuuugaagcucuauuuuaauaguauuuauuuuagaaagucuacuauuguaagaguucuucuguuugugaagaaaaaaacaaguuaaaaacugaauguacugauuuagaaaauauauauaaauauauauuguuaaauauacacgggacugccguu

SEQ ID NO: 6 - protein, 299 aa, Homo sapiens
MEPPIPQSAPLTPNSVMVQPLLDSRMSHSRLQHPLTILPIDQVKTSHVENDYIDNPSLALTTGPKRTRGGAPELAPTPARCDQDVTHHWISFSGRPSSVSSSSSTSSDQRLLDHMAPPPVADQASPRAVRIQPKVVHCQPLDLKGPAVPPELDKHFLLCEACGKCKCKECASPRTLPSCWVCNQECLCSAQTLVNYGTCMCLVQGIFYHCTNEDDEGSCADHPCSCSRSNCCARWSFMGALSVVLPCLLCYLPATGCVKLAQRGYDRLRRPGCRCKHTNSVICKAASGDAKTSRPDKPF

SEQ ID NO: 7 - RNA, 5625 nt, Homo sapiens
accuagcgcccccucccccgggagcgcggaggagcauuaauaaaccucuaagccgaggagaaaacucuggcuggggcagugcgcugagcgccggaggagcguaggcagggcagcgcuggcgccaguggcgacaggagccgcgcgaccggcaaaaauacacgggaggccgucgccgaaaagaguccgcgguccucucucguaaacacacucuccuccaccggcgccucccccuccgcucugcgcgccgcccggcugggcgcccgaggccgcuccgacugcuaugugaccgcgaggcugcgggaggaaggggacagggaagaagaggcucucccgcgggagcccuugaggaccaaguuugcggccacuucugcaggcgucccuucuuagcucucgcccgccccuuucugcagccuaggcggcccggguucucuucucuuccucgcgcgcccagccgccucgguucccggcgaccauggugacgauggaggagcugcgggagauggacugcagugugcucaaaaggcugaugaaccgggacgagaauggcggcggcgcgggcggcagcggcagccacggcacccuggggcugccgagcggcggcaagugccugcugcuggacugcagaccguuccuggcgcacagcgcgggcuacauccuagguucggucaacgugcgcuguaacaccaucgugcggcggcgggcuaagggcuccgugagccuggagcagauccugcccgccgaggaggagguacgcgcccgcuugcgcuccggccucuacucggcggucaucgucuacgacgagcgcagcccgcgcgccgagagccuccgcgaggacagcaccgugucgcugguggugcaggcgcugcgccgcaacgccgagcgcaccgacaucugccugcucaaaggcggcuaugagagguuuuccuccgaguacccagaauucuguucuaaaaccaaggcccuggcagccaucccacccccgguuccccccagugccacagagcccuuggaccugggcugcagcuccugugggaccccacuacacgaccagggggguccuguggagauccuucccuuccucuaccucggcagugccuaccaugcugcccggagagacaugcuggacgcccugggcaucacggcucuguugaaugucuccucggacugcccaaaccacuuugaaggacacuaucaguacaagugcaucccaguggaagauaaccacaaggccgacaucagcuccugguucauggaagccauagaguacaucgaugccgugaaggacugccgugggcgcgugcuggugcacugccaggcgggcaucucgcggucggccaccaucugccuggccuaccugaugaugaagaaacgggugaggcuggaggaggccuucgaguucguuaagcagcgccgcagcaucaucucgcccaacuucagcuucauggggcagcugcugcaguucgagucccaggugcuggccacguccugugcugcggaggcugcuagccccucgggaccccugcgggagcggggcaagacccccgccacccccaccucgcaguucgucuucagcuuuccggucuccgugggcgugcacucggcccccagcagccugcccuaccugcacagccccaucaccaccucucccagcuguuagagccgcccugggggccccagaaccagagcuggcucccagcaaggguaggacgggccgcaugcgggcagaaaguugggacugagcagcugggagcaggcgaccgagcuccuuccccaucauuucuccuuggccaacgacgaggccagccagaauggcaauaaggacuccgaauacauaauaaaagcaaacagaacacuccaacuuagagcaauaacggcugccgcagcagccagggaagaccuugguuugguuuaugugucaguuucacuuuuccgauagaaauuucuuaccucauuuuuuuaagcaguaaggcuugaagugaugaaacccacagauccuagcaaaugugcccaaccagcuuuacuaaagggggaggaagggagggcaaagggaugagaagacaaguuucccagaagugccugguucuguguacuugucccuuuguugucguuguuguaguuaaaggaauuucauuuuuuaaaagaaaucuucgaaggugugguuuucauuucucagucaccaacagaugaauaauuaugcuuaauaauaaaguauuuauuaagacuuucuucagaguaugaaaguacaaaaagucuaguuacaguggauuuagaauauauuuauguugaugucaaacagcugagcaccguagcaugcagaugucaaggcaguuaggaaguaaauggugucuuguagauaugugcaagguagcaugaugagcaacuugaguuuguugccacugagaagcaggcggguugggugggaggaggaagaaagggaagaauuagguuugaauugcuuuuuaaaaaaaaaagaaaagaaaaagacagcaucucacuauguugccaaggcucaucuugagaagcaggcggguugggugggaggaggaagaaagggaagaauuagguuugaauugcuuuuuuaaaaaaaaagaaaagaaaaaaaaagacagcaucucacuauguugccaaggcucaucucaagcucuugggcucaagagauccucccaccucggccuccugaguagcugggacugcaggugugugucaucaugaccaaugugaauugcuuuugaagcugguucaugggcauguaggccaccgaagcaauuuuagaccacaguaagucaagcuuuuuucccuccgaugaucacugggugguugcagcauuuuuugcauaaaccugccuaagacuugucuaucgucugugaucaauaugccauauuacacuaaggugcuccuggaaaauugggugcaguucaaauuuuccuacagcaaaucauuuggcaaggccagccauuggggaaaccagacaacuagagauaacccugaaaugaauccuuuuguaaauugaagcaccaucuuuucuuuuuuugcauaaauuggagguuuuaauuuuagggcaguuaccugaagugaaauauaccaacaauuucuuguguucuuuaaauuccuaguuaggugaauauuuuugaagguccucuuuugaauaaagaggggaauggacaccacauuucaggucuucucgaaguguggaagggcaagagagcaucagugagcugaugguggauugcuuacaucggauuccauugguaugaauuucccaaacuggaaaucaaagcgccagggugggguuggggcugacugcuggugagggggcuggccgcuggcucccgugacgugcgucaugggcacgcaggcgccauuuugaaucuaucgucggcacgugggugccauuuugaauccuuaguugggccuuucuaaauggagaauggcuuuggagggagacacguuuucuguggggaggguuugggggggagggaggagggaacaagcuacaugcuauuuuguuuguaguauuguggaacagucuuguuauggagugccagcuuagagguuguugcaaacuugucuagaagugagagcaugguuuuuuuuagcccuuugagagucuacaucuaaugaacauucuugcucacccauaaauaacgucaagccucaaugucaccgucacguugggauacucuuucucaucuggcauccuagacaggacaagguugguuaccuuuccuuccaugaaccaugaaccugugacggcaucauucauccugacuucaccaagcuccgccugugggugaggccagagcucccacuggcaauuuuuagaagagccagaggcucccugcuuccucuagaaauaacaguucagggugaagcauggaggguuucaguucccagacaauggaaccauuuagagacaacacaguuggacauuuccacuuuuuccuugauuccuggaaguccaguggguucugcagcugaaaaagcccugggucccagcagcagagagacaggacagaggggaugcuugggcggggagggacgguaaccugcagaacagauuccauuuuuauagaacgaguacacguuugcuaaaacaguccugcuuucccagacuggauucccaccacagggacagucggaacucaggacuagcuccagcgacaucuuuccuccgaauucaagccuucuaucacaaugucaaaacagcuauuuauaaagccauuuucauuguacuugauaacagcacgagucccaaaacuuuuagaaauaaaauaggacauuggcuugauugaaaagagggacuuuuuaaaaauuguucuuucgucagaagccuuuuggaugacuuacaauagcucugaugaagauaccaccccagcgucaguccaauaggucagugaguuucaacaggcauccaucccucccaugaagggauucuggugaugggaaguuucuguaaugacaggaaagcauugacccucauugauugucaacuuugguauuagccaugaaagacaggaugcucauuggguguucuguagagugaggaaugcugccuauucccucccagaacgucugacccagggguguguguugaggagcccugggggaaauggaccaaguuuucccacagagcaguauuaggcugaagagcaggugacugguaggccccagcucccaucauucccucccaaagccauuuuguucaguugcucauccacgcuggauuccagagaguuuuccaauuugggaagccaugagaaagguuuuuaaaucuugggaagauggagagagggacauaggauaguugacuccaacaugacaggaagaggcuggagauugggaauuggccaucaaccaagccuguaguaguaaagccauggucccgcauuggaauuacuuggggaacuuauacaguucugauacccaggcucuccuagaccaguucaaccaauucuaggugggggacucaggcaucaguguguuucguagcuccccggguguuuucccugugcagccgagcuugggaaacugccaugcuuuuuggaugucaaggcgcuguuggaggcugggugugacagcacagagccagguugucuuguggaaaccacagccacggguuugccacuggcucagcauggccucacugccagucccagccuggcugagggacaagaugguuucucuugggaguuccugaguggagcacccuuccaggcuuuuugaaagccagcugaucuguggagccuuguuaagggacucaauacgguguuuggauauugauguuuuuccuugagacugucuuguccaucaauaaagauggaggaugucuccucuuugaaccccgcuuccccaccaguacucucucucccuuagaguuuaugaguuauucaaggaggagacuucuuaaagacagcaacgcaauucuuguaacuuguguaaauagccccaucuuucagagugauaccauuucuacauuugauaaugccuguauuccuguaggauguauauaguuuaggggauuuuuuuuuuguuugguuuuguuuuuuagaagucaauaugucugguuuuauuuauugcuugaaaaagaucauuugaaaaaaauaaauacauuuucaaccacuaaaa

SEQ ID NO: 8 - protein, 394 aa, Homo sapiens
MVTMEELREMDCSVLKRLMNRDENGGGAGGSGSHGTLGLPSGGKCLLLDCRPFLAHSAGYILGSVNVRCNTIVRRRAKGSVSLEQILPAEEEVRARLRSGLYSAVIVYDERSPRAESLREDSTVSLVVQALRRNAERTDICLLKGGYERFSSEYPEFCSKTKALAAIPPPVPPSATEPLDLGCSSCGTPLHDQGGPVEILPFLYLGSAYHAARRDMLDALGITALLNVSSDCPNHFEGHYQYKCIPVEDNHKADISSWFMEAIEYIDAVKDCRGRVLVHCQAGISRSATICLAYLMMKKRVRLEEAFEFVKQRRSIISPNFSFMGQLLQFESQVLATSCAAEAASPSGPLRERGKTPATPTSQFVFSFPVSVGVHSAPSSLPYLHSPITTSPSC

SEQ ID NO: 9 - RNA, 2957 nt, Homo sapiens
agggcuuuugugcaugcuaauugcgccauuggugcgcaaaaguuccgacgcgaagugugaacucucaacagaaggaaacaugagacaacugaagugcccugguuuaugugcccugcuccuccuccgcugccgccuccccuucuuucuccuuccucccgccgagcccgcuguugcagcuuguuugcacuggggcuuauccggagcggaaauuccuuuccguuuuugugaaugacaaacucauuaacaauucaucaacacaaccuguuccagccggcccguccccacggcaacagccccuuccgcagcacgcucauuggcuggcccggagaauguauccauugagacgcucgcuguuuguauccauugaggagcugccucgcgcagggggugugcgaggcugaguccaagagauagcaaaucgagucuuaaauaauccggggagaaagacgcccggguagauuugaggugcagccuuggagggagggauuagaagccgcuagacuuuuuuuccuccccucucaguagcacggaguccgaauuaauuggauuucauucacuggggaggaacaaaaacuaucugggcagcuucauugagagagauucauugacacuaagagccagcggcugcagcugggugcagagagaaccuccggcuuuacuucugucucgucugccccaaccgcuagccucggcuuggguaaggcgaggcggaauuaaaccccgcuccgagagcggcagcuucgcgcgcggugcgcucggccuaugccugccccgaggggcgucugguaggcaccccgcccucucccgcagcucgacccccaugauagauacgcucagacccgugcccuucgcgucggaaauggcgaucagcaagacgguggcguggcucaacgagcagcuggagcugggcaacgagcggcugcugcugauggacugccggccgcaggagcuauacgagucgucgcacaucgagucggccaucaacguggccaucccgggcaucaugcugcggcgccugcagaaggguaaccugccggugcgcgcgcucuucacgcgcggcgaggaccgggaccgcuucacccggcgcuguggcaccgacacaguggugcucuacgacgagagcagcagcgacuggaacgagaauacgggcggcgagucggugcucgggcugcugcucaagaagcucaaggacgagggcugccgggcguucuaccuggaagaugaagcccggggcaagaacuguggugucuugguacauugcuuggcuggcauuagccgcucagucacugugacuguggcuuaccuuaugcagaagcucaaucugucgaugaacgaugccuaugacauugucaaaaugaaaaaauccaacauauccccuaacuucaacuucaugggucagcugcuggacuucgagaggacgcugggacucagcagcccaugugacaacaggguuccagcacagcagcuguauuuuaccaccccuuccaaccagaauguauaccagguggacucucugcaaucuacgugaaagaccccacaccccuccuugcuggaaugugucuggcccuucagcaguuucucuuggcagcaucagcugggcugcuuucuuuguguguggccccaggugucaaaaugacaccagcugucuguacuagacaagguuaccaagugcggaauugguuaauacuaacagagagauuugcuccauucucuuuggaauaacaggacaugcuguauagauacaggcaguagguuugcucuguacccauguguacagccuacccaugcagggacugggauucgaggacuuccaggcgcauaggguagaaccaaaugauaggguaggagcauguguucuuuagggccuuguaaggcuguuuccuuuugcaucuggaacugacuauauaauugucuucaaugaagacuaauucaauuuugcauauagaggagccaaagagagauuucagcucuguauuugugguaucaguuuggaaaaaaaaaucugauacuccauuugauuauuguaaauauuugaucuugaaucacuugacaguguuuguuugaauuguguuuguuuuuuccuuugaugggcuuaaaagaaauuauccaaagggagaaagagcaguaugccacuucuuaaaacagaacaaaacaaaaaaagaaaauugugcucuuuucuaauccaaaggguauauuugcagcaugcuugacuuuaccaauucugaugacaucuuuacggacacuauuaucacuaagaccuuguuauggcgaagucuuuagucuuuuucauguauuuuccucaugauuuuuucucuuuauguaguuugacuaugccuuaccuuuguaaauauuuuugcuuguguugucgcaaaggggauaaucugggaaagacaccaaaucaugggcucacuuuaaaaaaagaaagaauaaaaaaaccuucagcugugcuaaacaguauauuaccucuguauaaaauucuucagggagugucaccucaaaugcaauacuuuggguugguuucuuuccuuuaaaaaaauuuguauaaaacuggaagugugugugugugagcauggguacccauuugauaagagaaaugcauuugauugugaagaagggagaguuaaauucuccauuauguucgugguguaaaguuuagagcuggaauuuauuauaagaauguaaaaccuuaaauuauuaauaaauaacuauuuuggcuauugaauguguguuuuuaaaaacucaguuauaccuaugugugaagugacacauuuuagaacuuuuucauucagggauugauuuugacuuguaggcauaguagggcaagguucuuuuaagcuuuuccuagaacguuuuucagcagcaguuuaugaaauucaucguuugccacgaauaaagaugacauccuagaauggaccgacuguaccguguauaugaacaauaccucuuaugaguauagu

SEQ ID NO: 10 - protein, 381 aa, Homo sapiens
MIDTLRPVPFASEMAISKTVAWLNEQLELGNERLLLMDCRPQELYESSHIESAINVAIPGIMLRRLQKGNLPVRALFTRGEDRDRFTRRCGTDTVVLYDESSSDWNENTGGESVLGLLLKKLKDEGCRAFYLEGGFSKFQAEFSLHCETNLDGSCSSSSPPLPVLGLGGLRISSDSSSDIESDLDRDPNSATDSDGSPLSNSQPSFPVEILPFLYLGCAKDSTNLDVLEEFGIKYILNVTPNLPNLFENAGEFKYKQIPISDHWSQNLSQFFPEAISFIDEARGKNCGVLVHCLAGISRSVTVTVAYLMQKLNLSMNDAYDIVKMKKSNISPNFNFMGQLLDFERTLGLSSPCDNRVPAQQLYFTTPSNQNVYQVDSLQST

SEQ ID NO: 11 - RNA, 4304 nt, Homo sapiens
cacacggacuacaggggaguuuuguugaaguugcaaaguccuggagccuccagagggcugucggcgcaguagcagcgagcagcagaguccgcacgcuccggcgaggggcagaagagcgcgagggagcgcggggcagcagaagcgagagccgagcgcggacccagccaggacccacagcccuccccagcugcccaggaagagccccagccauggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucugagggcgccaggcaggcgggcgccaccgccacccgcagcgagggcggagccggccccaggugcuccccugacagucccuccucuccggagcauuuugauaccagaagggaaagcuucauucuccuuguuguugguuguuuuuuccuuugcucuuucccccuuccaucucugacuuaagcaaaagaaaaagauuacccaaaaacugucuuuaaaagagagagagagaaaaaaaaaauaguauuugcauaacccugagcggugggggaggaggguugugcuacagaugauagaggauuuuauaccccaauaaucaacucguuuuuauauuaauguacuuguuucucuguuguaagaauaggcauuaacacaaaggaggcgucucgggagaggauuagguuccauccuuuacguguuuaaaaaaaagcauaaaaacauuuuaaaaacauagaaaaauucagcaaaccauuuuuaaaguagaagaggguuuuagguagaaaaacauauucuugugcuuuuccugauaaagcacagcuguagugggguucuaggcaucucuguacuuugcuugcucauaugcauguagucacuuuauaagucauuguauguuauuauauuccguagguagauguguaaccucuucaccuuauucauggcugaagucaccucuugguuacaguagcguagcgugcccgugugcauguccuuugcgccugugaccaccaccccaacaaaccauccagugacaaaccauccaguggagguuugucgggcaccagccagcguagcagggucgggaaaggccaccugucccacuccuacgauacgcuacuauaaagagaagacgaaauagugacauaauauauucuauuuuuauacucuuccuauuuuuguagugaccuguuuaugagaugcugguuuucuacccaacggcccugcagccagcucacguccagguucaacccacagcuacuugguuuguguucuucuucauauucuaaaaccauuccauuuccaagcacuuucaguccaauagguguaggaaauagcgcuguuuuuguugugugugcagggagggcaguuuucuaauggaaugguuugggaauauccauguacuuguuugcaagcaggacuuugaggcaagugugggccacugugguggcaguggaggugggguguuugggaggcugcgugccagucaagaagaaaaagguuugcauucucacauugccaggaugauaaguuccuuuccuuuucuuuaaagaaguugaaguuuaggaauccuuuggugccaacugguguuugaaaguagggaccucagagguuuaccuagagaacaggugguuuuuaaggguuaucuuagauguuucacaccggaagguuuuuaaacacuaaaauauauaauuuauaguuaaggcuaaaaaguauauuuauugcagaggauguucauaaggccaguaugauuuauaaaugcaaucuccccuugauuuaaacacacagauacacacacacacacacacacacacaaaccuucugccuuugauguuacagauuuaauacaguuuauuuuuaaagauagauccuuuuauaggugagaaaaaaacaaucuggaagaaaaaaaccacacaaagacauugauucagccuguuuggcguuucccagagucaucugauuggacaggcaugggugcaaggaaaauuaggguacucaaccuaaguucgguuccgaugaauucuuauccccugccccuuccuuuaaaaaacuuagugacaaaauagacaauuugcacaucuuggcuauguaauucuuguaauuuuuauuuaggaaguguugaagggagguggcaagaguguggaggcugacgugugagggaggacaggcgggaggaggugugaggaggaggcucccgaggggaaggggcggugcccacaccggggacaggccgcagcuccauuuucuuauugcgcugcuaccguugacuuccaggcacgguuuggaaauauucacaucgcuucuguguaucucuuucacauuguuugcugcuauuggaggaucaguuuuuuguuuuacaaugucauauacugccauguacuaguuuuaguuuucucuuagaacauuguauuacagaugccuuuuuuguaguuuuuuuuuuuuuuaugugaucaauuuugacuuaaugugauuacugcucuauuccaaaaagguugcuguuucacaauaccucaugcuucacuuagccaugguggacccagcgggcagguucugccugcuuuggcgggcagacacgcgggcgcgaucccacacaggcuggcgggggccggccccgaggccgcgugcgugagaaccgcgccgguguccccagagaccaggcugugucccucuucucuucccugcgccugugaugcugggcacuucaucugaucgggggcguagcaucauaguaguuuuuacagcuguguuauucuuugcguguagcuauggaaguugcauaauuauuauuauuauuauuauaacaagugugucuuacgugccaccacggcguuguaccuguaggacucucauucgggaugauuggaauagcuucuggaauuuguucaaguuuuggguauguuuaaucuguuauguacuaguguucuguuuguuauuguuuuguuaauuacaccauaaugcuaauuuaaagagacuccaaaucucaaugaagccagcucacagugcugugugccccggucaccuagcaagcugccgaaccaaaagaauuugcaccccgcugcgggcccacgugguuggggcccugcccuggcagggucauccugugcucggaggccaucucgggcacaggcccaccccgccccaccccuccagaacacggcucacgcuuaccucaaccauccuggcugcggcgucugucugaaccacgcgggggccuugagggacgcuuugucugucgugauggggcaagggcacaaguccuggauguuguguguaucgagaggccaaaggcugguggcaagugcacggggcacagcggagucuguccugugacgcgcaagucugagggucugggcggcgggcggcugggucugugcauuucugguugcaccgcggcgcuucccagcaccaacauguaaccggcauguuuccagcagaagacaaaaagacaaacaugaaagucuagaaauaaaacugguaaaaccccaaaaaaaaaaaaaaaa

SEQ ID NO: 12 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 13 - RNA, 3970 nt, Homo sapiens
gguucucacccaacuuccauuaaggacucggggcaggaggggcagaaguugcgcgcaggccggcgggcgggagcggacaccgaggccggcgugcaggcgugcgggugugcgggagccgggcucggggggaucggaccgagagcgagaagcgcggcauggagcuccaggcagcccgcgccugcuucgcccugcuguggggcugugcgcuggccgcggccgcggcggcgcagggcaaggaagugguacugcuggacuuugcugcagcuggaggggagcucggcuggcucacacacccguauggcaaagggugggaccugaugcagaacaucaugaaugacaugccgaucuacauguacuccgugugcaacgugaugucuggcgaccaggacaacuggcuccgcaccaacuggguguaccgaggagaggcugagcguaucuucauugagcucaaguuuacuguacgugacugcaacagcuucccugguggcgccagcuccugcaaggagacuuucaaccucuacuaugccgagucggaccuggacuacggcaccaacuuccagaagcgccuguucaccaagauugacaccauugcgcccgaugagaucaccgucagcagcgacuucgaggcacgccacgugaagcugaacguggaggagcgcuccguggggccgcucacccgcaaaggcuucuaccuggccuuccaggauaucggugccuguguggcgcugcucuccguccgugucuacuacaagaagugccccgagcugcugcagggccuggcccacuucccugagaccaucgccggcucugaugcaccuucccuggccacuguggccggcaccuguguggaccaugccguggugccaccggggggugaagagccccguaugcacugugcaguggauggcgaguggcuggugcccauugggcagugccugugccaggcaggcuacgagaagguggaggaugccugccaggccugcucgccuggauuuuuuaaguuugaggcaucugagagccccugcuuggagugcccugagcacacgcugccauccccugagggugccaccuccugcgagugugaggaaggcuucuuccgggcaccucaggacccagcgucgaugccuugcacacgaccccccuccgccccacacuaccucacagccgugggcaugggugccaagguggagcugcgcuggacgcccccucaggacagcgggggccgcgaggacauugucuacagcgucaccugcgaacagugcuggcccgagucuggggaaugcgggccgugugaggccagugugcgcuacucggagccuccucacggacugacccgcaccagugugacagugagcgaccuggagccccacaugaacuacaccuucaccguggaggcccgcaauggcgucucaggccugguaaccagccgcagcuuccguacugccagugucagcaucaaccagacagagccccccaaggugaggcuggagggccgcagcaccaccucgcuuagcgucuccuggagcauccccccgccgcagcagagccgaguguggaaguacgaggucacuuaccgcaagaagggagacuccaacagcuacaaugugcgccgcaccgaggguuucuccgugacccuggacgaccuggccccagacaccaccuaccugguccaggugcaggcacugacgcaggagggccagggggccggcagcaaggugcacgaauuccagacgcuguccccggagggaucuggcaacuuggcggugauuggcggcguggcugucggugugguccugcuucuggugcuggcaggaguuggcuucuuuauccaccgcaggaggaagaaccagcgugcccgccaguccccggaggacguuuacuucuccaagucagaacaacugaagccccugaagacauacguggacccccacacauaugaggaccccaaccaggcuguguugaaguucacuaccgagauccauccauccugugucacucggcagaaggugaucggagcaggagaguuuggggagguguacaagggcaugcugaagacauccucggggaagaaggaggugccgguggccaucaagacgcugaaagccggcuacacagagaagcagcgaguggacuuccucggcgaggccggcaucaugggccaguucagccaccacaacaucauccgccuagagggcgucaucuccaaauacaagcccaugaugaucaucacugaguacauggagaauggggcccuggacaaguuccuucgggagaaggauggcgaguucagcgugcugcagcuggugggcaugcugcggggcaucgcagcuggcaugaaguaccuggccaacaugaacuaugugcaccgugaccuggcugcccgcaacauccucgucaacagcaaccuggucugcaaggugucugacuuuggccugucccgcgugcuggaggacgaccccgaggccaccuacaccaccaguggcggcaagauccccauccgcuggaccgccccggaggccauuuccuaccggaaguucaccucugccagcgacguguggagcuuuggcauugucaugugggaggugaugaccuauggcgagcggcccuacugggaguuguccaaccacgaggugaugaaagccaucaaugauggcuuccggcuccccacacccauggacugccccuccgccaucuaccagcucaugaugcagugcuggcagcaggagcgugcccgccgccccaaguucgcugacaucgucagcauccuggacaagcucauucgugccccugacucccucaagacccuggcugacuuugacccccgcgugucuauccggcuccccagcacgagcggcucggagggggugcccuuccgcacgguguccgaguggcuggaguccaucaagaugcagcaguauacggagcacuucauggcggccggcuacacugccaucgagaagguggugcagaugaccaacgacgacaucaagaggauuggggugcggcugcccggccaccagaagcgcaucgccuacagccugcugggacucaaggaccaggugaacacuguggggauccccaucugagccucgacagggccuggagccccaucggccaagaauacuugaagaaacagaguggccucccugcugugccaugcugggccacuggggacuuuauuuauuucuaguucuuuccucccccugcaacuuccgcugaggggucucggaugacacccuggccugaacugaggagaugaccagggaugcugggcugggcccucuuucccugcgagacgcacacagcugagcacuuagcaggcaccgccacgucccagcaucccuggagcaggagccccgccacagccuucggacagacauaugggauauucccaagccgaccuucccuccgccuucucccacaugaggccaucucaggagauggagggcuuggcccagcgccaaguaaacaggguaccucaagccccauuuccucacacuaagagggcagacugugaacuugacugggugagacccaaagcggucccugucccucuagugccuucuuuagacccucgggccccauccucaucccugacuggccaaacccuugcuuuccugggccuuugcaagaugcuugguuguguugagguuuuuaaauauauauuuuguacuuuguggagagaauguguguguguggcagggggccccgccagggcuggggacagagggugucaaacauucgugagcuggggacucagggaccggugcugcaggaguguccugcccaugccccagucggccccaucucucauccuuuuggauaaguuucuauucugucaguguuaaagauuuuguuuuguuggacauuuuuuucgaaucuuaauuuauuauuuuuuuuauauuuauuguuagaaaaugacuuauuucugcucuggaauaaaguugcagaugauucaaaccgaaaaaaa

SEQ ID NO: 14 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 15 - RNA, 6597 nt, Homo sapiens
acgcgugcucaucuuguguaaaaguaaaaguugcucaugucauuagcucggcucugaguuugcaugagaaaccagcgcagcucucgagccacgggagaaaacacgaucugcugcggagcccagugaccugaaacuguagcagugacucgagaccuguccucuccucgacgucgcccuauaacccucccgcuuuauuuaggauccgcuuuuccgagagcagccacucaggcaacccccggaagaagcggcaggagcagcguuggcaccggcgaaccauggcugggauuuucuauuucgcccuauuuucgugucucuucgggauuugcgacgcugucacagguuccaggguauaccccgcgaaugaaguuaccuuauuggauuccagaucuguucagggagaacuuggguggauagcaagcccucuggaaggagggugggaggaagugaguaucauggaugaaaaaaauacaccaauccgaaccuaccaagugugcaaugugauggaacccagccagaauaacuggcuacgaacugauuggaucacccgagaaggggcucagaggguguauauugagauuaaauucaccuugagggacugcaauagucuuccgggcgucauggggacuugcaaggagacguuuaaccuguacuacuaugaaucagacaacgacaaagagcguuucaucagagagaaccaguuugucaaaauugacaccauugcugcugaugagagcuucacccaaguggacauuggugacagaaucaugaagcugaacaccgagauccgggauguagggccauuaagcaaaaagggguuuuaccuggcuuuucaggaugugggggccugcaucgcccugguaucaguccguguguucuauaaaaaguguccacucacaguccgcaaucuggcccaguuuccugacaccaucacaggggcugauacgucuucccugguggaaguucgaggcuccugugucaacaacucagaagagaaagaugugccaaaaauguacuguggggcagauggugaauggcugguacccauuggcaacugccuaugcaacgcugggcaugaggagcggagcggagaaugccaagcuugcaaaauuggauauuacaaggcucucuccacggaugccaccugugccaagugcccaccccacagcuacucugucugggaaggagccaccucgugcaccugugaccgaggcuuuuucagagcugacaacgaugcugccucuaugcccugcacccguccaccaucugcuccccugaacuugauuucaaaugucaacgagacaucugugaacuuggaauggaguagcccucagaauacagguggccgccaggacauuuccuauaaugugguaugcaagaaauguggagcuggugaccccagcaagugccgacccuguggaagugggguccacuacaccccacagcagaauggcuugaagaccaccaaagucuccaucacugaccuccuagcucauaccaauuacaccuuugaaaucugggcugugaauggaguguccaaauauaacccuaacccagaccaaucaguuucugucacugugaccaccaaccaagcagcaccaucauccauugcuuugguccaggcuaaagaagucacaagauacaguguggcacuggcuuggcuggaaccagaucggcccaaugggguaauccuggaauaugaagucaaguauuaugagaaggaucagaaugagcgaagcuaucguauaguucggacagcugccaggaacacagauaucaaaggccugaacccucucacuuccuauguuuuccacgugcgagccaggacagcagcuggcuauggagacuucagugagcccuuggagguuacaaccaacacagugccuucccggaucauuggagauggggcuaacuccacaguccuucuggucucugucucgggcaguguggugcuggugguaauucucauugcagcuuuugucaucagccggagacggaguaaauacaguaaagccaaacaagaagcggaugaagagaaacauuugaaucaagguguaagaacauauguggaccccuuuacguacgaagaucccaaccaagcagugcgagaguuugccaaagaaauugacgcauccugcauuaagauugaaaaaguuauaggaguuggugaauuuggugagguaugcagugggcgucucaaagugccuggcaagagagagaucuguguggcuaucaagacucugaaagcugguuauacagacaaacagaggagagacuuccugagugaggccagcaucaugggacaguuugaccauccgaacaucauucacuuggaaggcguggucacuaaauguaaaccaguaaugaucauaacagaguacauggagaauggcuccuuggaugcauuccucaggaaaaaugauggcagauuuacagucauucagcuggugggcaugcuucguggcauugggucugggaugaaguauuuaucugauaugagcuaugugcaucgugaucuggccgcacggaacauccuggugaacagcaacuuggucugcaaagugucugauuuuggcaugucccgagugcuugaggaugauccggaagcagcuuacaccaccagggguggcaagauuccuauccgguggacugcgccagaagcaauugccuaucguaaauucacaucagcaagugauguauggagcuauggaaucguuaugugggaagugaugucguacggggagaggcccuauugggauauguccaaucaagaugugauuaaagccauugaggaaggcuaucgguuacccccuccaauggacugccccauugcgcuccaccagcugaugcuagacugcuggcagaaggagaggagcgacaggccuaaauuugggcagauugucaacauguuggacaaacucauccgcaaccccaacagcuugaagaggacagggacggagagcuccagaccuaacacugccuuguuggauccaagcuccccugaauucucugcugugguaucagugggcgauuggcuccaggccauuaaaauggaccgguauaaggauaacuucacagcugcugguuauaccacacuagaggcuguggugcacgugaaccaggaggaccuggcaagaauugguaucacagccaucacgcaccagaauaagauuuugagcaguguccaggcaaugcgaacccaaaugcagcagaugcacggcagaaugguucccgucugagccaguacugaauaaacucaaaacucuugaaauuaguuuaccucauccaugcacuuuaauugaagaacugcacuuuuuuuacuucgucuucgcccucugaaauuaaagaaaugaaaaaaaaaaacaauaucugcagcguugcuuggugcacagauugcugaaacuguggggcuuacagaaaugacugccggucauuugaaugagaccuggaacaaaucguuucucagaaguacuuuucuguucaucaccagucuguaaaauacauguaccuauagaaauagaacacugccucugaguuuugaugcuguauuugcugccagacacugagcuucugagacaucccugauucucucuccauuuggaauuacaaccauuguauuuuguuuguggcauaaauuacagucaucugucuuucacuggaaugaagaccaugccuaggaacauuuuuuaaggacucagcuguggcuuuuagggcuugguucauaccaugggggaaaaaaaaguccuaggagaaagcgacguggcucauuaguguugccucuucagugcucaagccgccugguggauuccuaugacacagggggccuggaaagaaagggaaaguggauuuaaaauauauauauacguaacccaagccccauaaccccuaacuggacaaaugaggucuguuucuuugggccugaggcugugccauauaaagucuuauuuugggacuuuacaaacuuguccuaacuaucuuguggauagugggcugugacaaucuggaauagagaacguucacacuucgcuccuuuaaagaagcgaccccagaucugcaagggaguagauucugcuaucuuggccucacagcccuuccuguugauuacaaagcccguggaagaaaacagaacacacccuccucaguuccgucuaaauguguuucuucugcuucaauuacaccaguucuggggcaaagacacugaugaaacaacacccauaccugaaaagaauaaaugugugacuuucaaauccccuuucgcagugaaagaaacagcaaacacuuaagauucagcaucuguucuccaguugcacugaggaaugcacugucucgcagcaccagcucugcagagcccuugccccagacucuuugcgguuuuauuuauauguauuuccauauuucauuccugugugucacugcugcauugguguggcagcaagugaccaaaugcuacaggucuuacuauggacaccaggucaggugcaaccacacaaaacaaagccaguuccaugagcugccuaugauaugcauugcggaaguaacauuuuacccagggugugccauugcagugauauaaauauauuuuuuucuuagacuaaauaugagcugacuaucucuuuugauguguguacauaggugugagugugucuguaugcgugccugucugugugcggguguguguaugugcguagccucaugcuuaggacuacccaugaauguuguggaaugcuacaccuggagaguucugguuuuccaccaguuucaagaugaagaacuacaugauacaguggaccuggagaccauccccuuggaaagacaacccagagauguucagcauccuguaucuacacgcauccuguaucuacacguguauuuuguagcugucacacuaaccuuaauaagaauucuacagcuuuggacagaggcauuuucaccuuaauggugaaguaauuuaaaauauaaauccauucaggugacaacccaucaucaaaauuacaaauuuucugauugaacucaucugaaucaucaguuccuugauggagagagagaaggagauggaaugugucugguaaccccaaauggaguacaaguagccuuuguuuuccugcauaaauggacuuguugaaugcgaacgaauauaugcaauucauauacuuuuggagaugaacguagauaugugugucagcuuugagaugguguguccuggauuaauacuuugucucccaauaucacagaaaaauacaugccagugacucuugagguuaagguaguugggaugaaauggccucaggcaauuucacauucccuaauuaccuggaaaguucuacaguaauuaauaugcagcuaacuccuguugcccucacaagagcaucagccuucuagaaucggagcuccggagugugaagauucaguauugauaugauauguauaccaaacuccagccaacuuacugccauuuuucauaaucugaguggcugccuugcuuauccuaagcugugguugcagaaaccguggccauuuauauaagcuauaacaucaaaucagggaaaaaugaggaaaaaaaauagauucugaaccauuuauuguugaauaaguagagaaaaucaucaauaaauauuuauuacauucugacaggguguguggcauuguguucuaugccagagugacaaaguugauucaccccuuuuuggggaccuuaauauauuuuuuaagggaugugccuaugcauugaugccugaaaaauauguauaaagaaaugagguugacucuucugagcaguucaucuuuuccagagguaaggguaggaggccaacuucagggucugggucugagcccgugggcaagcccuggccgagugagcuccaaugcuaacucaugugccgaucucuagagcagugggaaacuaccccgcugcaccaaaucaaguagcuucaccuuguguaugcaggccccaaguuauuuuuuagcaaucuuacgagugaaauguucugguggguugaaaaacguucuuauuuuaaagaaagguugugcucgcuacacugcuggugugugcauucugagaccucuuguauucaaucugugaaggauauguguauuaauccguacacccguauagccucaauauuugucugaagacacuuaaauucugacccauaaaggaaaguucuagaagcaauauuuucacuuauuuaacauucuccaaacaacaucaagcauugauacacacugaagagugcguuuauuuuuuguaucacucuaaguauguuggaauaugcaaggacugugguucaaauuagaauguauaaggcauauuauaauuuaguucauacugaauaagaaauuaacagaacauuguucgguucacacguuccaaacuuugagugauuucuggaguuagacauagauuuucuauuuuguuuuaauuugucaagguauuuuucuucccuucaugaacuuuagguacacauaacuuaugucauuuauuuauggucuuuuauaccuaguuuguaaaauuguaaaauagcaaacuaaaugcaaagaguuugcauuugaaaauaauaaaguaguugccguauacaacccugcaaaaaaaaaaaaaaaaaa

SEQ ID NO: 16 - protein, 986 aa, Homo sapiens
MAGIFYFALFSCLFGICDAVTGSRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQEDLARIGITAITHQNKILSSVQAMRTQMQQMHGRMVPV

SEQ ID NO: 17 - RNA, 2358 nt, Homo sapiens
gaaacaaagggaaacggggggcgguguggcggggcgggggguggagagcugaggccgagcgaaggaaaugcaccaaucagcugcucccccgggcucacaacugucugcugcgcccgaaaaacaagucggugcgcuggggacccggggccggggccgccuuacuccggccuagccccgcggcccucggugcgggcuccagggcaugcucgggaccccccgcggcuccagcccagacgccccggccucagaaaucgcccggaaaugggagcuugcgcgaagcgcugaucggcccgcuggggaagcucauggacccgggcucccugccgccccucgacucugaagaucucuuccaggaucuaagucacuuccaggagacguggcucgcugaagcucagguaccagacagugaugagcaguuuguuccugauuuccauucagaaaaccuagcuuuccacagccccaccaccaggaucaagaaggagccccagaguccccgcacagacccggcccuguccugcagcaggaagccgccacuccccuaccaccauggcgagcagugccuuuacuccagugccuaugacccccccagacaaaucgccaucaaguccccugccccuggugcccuuggacagucgccccuacagcccuuuccccgggcagagcaacggaauuuccugagauccucuggcaccucccagccccacccuggccauggguaccucggggaacauagcuccgucuuccagcagccccuggacauuugccacuccuucacaucucagggagggggccgggaaccccucccagcccccuaccaacaccagcugucggagcccugcccacccuauccccagcagagcuuuaagcaagaauaccaugauccccuguaugaacaggcgggccagccagccguggaccaggguggggucaaugggcacagguacccaggggcggggguggugaucaaacaggaacagacggacuucgccuacgacucagaugucaccgggugcgcaucaauguaccuccacacagagggcuucucugggcccucuccaggugacggggccaugggcuauggcuaugagaaaccucugcgaccauucccagaugaugucugcguugucccugagaaauuugaaggagacaucaagcaggaaggggucggugcauuucgagaggggccgcccuaccagcgccggggugcccugcagcuguggcaauuucugguggccuugcuggaugacccaacaaaugcccauuucauugccuggacgggccggggaauggaguucaagcucauugagccugaggaggucgccaggcucuggggcauccagaagaaccggccagccaugaauuacgacaagcugagccgcucgcuccgauacuauuaugagaaaggcaucaugcagaagguggcuggugagcguuacguguacaaguuugugugugagcccgaggcccucuucucuuuggccuucccggacaaucagcguccagcucucaaggcugaguuugaccggccugucagugaggaggacacagucccuuugucccacuuggaugagagccccgccuaccucccagagcuggcuggccccgcccagccauuuggccccaaggguggcuacucuuacuagcccccagcggcuguucccccugccgcaggugggugcugcccuguguacauauaaaugaaucugguguuggggaaaccuucaucugaaacccacagaugucucuggggcagauccccacuguccuaccaguugcccuagcccagacucugagcugcucaccggagucauugggaaggaaaaguggagaaauggcaagucuagagucucagaaacuccccuggggguuucaccugggcccuggaggaauucagcucagcuucuuccuagguccaagccccccacaccuuuuccccaaccacagagaacaagaguuuguucuguucugggggacagagaaggcgcuucccaacuucauacuggcaggagggugaggagguucacugagcuccccagaucucccacugcggggagacagaagccuggacucugccccacgcuguggcccuggagggucccgguuugucaguucuuggugcucuguguucccagaggcaggcggagguugaagaaaggaaccugggaugaggggugcuggguauaagcagagagggauggguuccugcuccaagggacccuuugccuuucuucugcccuuuccuaggcccaggccuggguuuguacuuccaccuccaccacaucugccagaccuuaauaaaggcccccacuucucccauuaaaaaaaaaaaaaaaaaaaaaaaaaaaaaaaaaaaaaaaa

SEQ ID NO: 18 - protein, 484 aa, Homo sapiens
MERRMKAGYLDQQVPYTFSSKSPGNGSLREALIGPLGKLMDPGSLPPLDSEDLFQDLSHFQETWLAEAQVPDSDEQFVPDFHSENLAFHSPTTRIKKEPQSPRTDPALSCSRKPPLPYHHGEQCLYSSAYDPPRQIAIKSPAPGALGQSPLQPFPRAEQRNFLRSSGTSQPHPGHGYLGEHSSVFQQPLDICHSFTSQGGGREPLPAPYQHQLSEPCPPYPQQSFKQEYHDPLYEQAGQPAVDQGGVNGHRYPGAGVVIKQEQTDFAYDSDVTGCASMYLHTEGFSGPSPGDGAMGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 19 - RNA, 4102 nt, Homo sapiens
agccugguuggcagcugcggcgcagaguccagccgcuggugcgcggagcgguucaccgucuucggagcgguucggcccagccuuucgcccaggcgcccaggcccgcugcgcgcgugcgugagcgcgccugcgccgccggggccgcugcaaggggaggagagaggccgccucaggaggaucccuuuucccccagaaauuacucaaugcugaaaccucucaaagugguauuagagacgcugaaagcaccauggacggguuuuaugaucagcaagucccuuuuauggucccagggaaaucucgaucugaggaaugcagagggcggccugugauugacagaaagaggaaguuuuuggacacagaucuggcucacgauucugaagagcuauuucaggaucucagucaacuucaagaggcuugguuagcugaagcacaaguuccugaugaugaacaguuugucccagauuuucagucugauaaccuggugcuucaugccccaccuccaaccaagaucaaacgggagcugcacagccccuccucugagcugucgucuuguagccaugagcaggcucuuggugcuaacuauggagaaaagugccucuacaacuauugugccuaugauaggaagccucccucuggguucaagccauuaaccccuccuacaaccccccucucacccacccaucagaauccccuauuucccccaccucaggcaacucugcccaccucagggcaugccccugcagcuggcccaguucaaggugugggccccgcccccgccccccauucgcuuccagagccuggaccacagcagcaaacauuugcggucccccgaccaccacaucagccccugcagaugccaaagaugaugccugaaaaccaguauccaucagaacagagauuucagagacaacugucugaacccugccaccccuucccuccucagccaggaguuccuggagauaaucgccccaguuaccaucggcaaaugucagaaccuauugucccugcagcucccccgcccccucagggauucaaacaagaauaccaugacccacucuaugaacauggggucccgggcaugccagggcccccagcacacggguuccagucaccaaugggaaucaagcaggagccucgggauuacugcgucgauucagaagugccuaacugccagucauccuacaugagaggggguuauuucuccagcagccaugaagguuuuucauaugaaaaagauccccgauuauacuuugacgacacuuguguugugccugagagacuggaaggcaaagucaaacaggagccuaccauguaucgagaggggcccccuuaccagaggcgagguucccuucagcuguggcaguuccuggucacccuucuugaugacccagccaaugcccacuucauugccuggacaggucgaggcauggaguucaagcugauagaaccggaagagguugcucggcgcuggggcauccagaagaaccggccagccaugaacuaugacaagcugagccgcucucuccgcuauuacuaugaaaagggcaucaugcagaagguggcuggagagcgauacgucuacaaauuugucugugacccagaugcccucuucuccauggcuuucccggauaaccagcguccguuccugaaggcagaguccgagugccaccucagcgaggaggacacccugccgcugacccacuuugaagacagccccgcuuaccuccuggacauggaccgcugcagcagccuccccuaugccgaaggcuuugcuuacuaaguuucugaguggcggaguggccaaacccuagagcuagcaguucccauucaggcaaacaagggcagugguuuuguuuguguuuuugguuguuccuaaagcuugcccuuugaguauuaucuggagaacccaagcugucucuggauuggcacccuuaaagacagauacauuggcuggggagugggaacagggaggggcagaaaaccaccaaaaggccagugccucaacucuugauucugaugagguuucugggaagagaucaaaauggagucuccuuaccauggacaauacaugcaaagcaauaucuuguucagguuaguacccgcaaaacgggacauaguaugugacaaucugcaucgaucauggacuacuaaaugccuuuacauagaagggcucugauuugcacaauuuguugaaaaaucacaaacccauagaaaaguaaguaggcuaaguuggggaggcucaaaccauuaaggguuaaaaauacaucuuaaacauuggaaagcucuucuagcugaaucugaaauauuaccccuugucuagaaaaaggggggcagucagaacagcuguuccccacuccgugguucucaaaaucauaaaccauggcuacucuugggaaccacccggccauguggucgccaaguagagcaagcccccuuucucuucccaaucacguggcugaguguggaugacuuuuauuuuaggagaagggcgauuaacacuuuugacaguauuuuguuuugcccugauuugggggauuguuuuguuuuggugguuguuuuggaaaaacaguuuauaaacugauuuuuguaguuuugguauuuaaagcaaaaaaacgaaaaacaaaaaacaaaaacaaaccuuuugguaacugugcacuguguccuuuagccagggccgugccaacuuaugaagacacugcagcuugagaggggcuuugcugaggcuuccccuuggccaugugaaagcccgccuuguugccugcuuugugcuuucugcaccagacaaccugauggaacauuugcaccugaguuguacauuuuugaagugugcagggcagccuggacacaagcuuagauucucuauguauaguuccccguguucacuaacaugcccucucuggaaagcauauguauauaacaugugucauguccuuuggaaaccuggucaccuggugaaaacccuugggauucuucccugggcaugacugaugacaauuuccauuucaucaguuuguuuuguuuuccuuuuucuuuaaaucuuggacuuuaaacccuaccugugugauucaguaggguuugagacuuacgugugauacugacagguaagcaacagugcuagcauucuagauuccugccuuuuuuuaaaaagaaauuauucucauugcuguauuauauuggaaaaguuuuaaacaaccaagcuaaagcuaugugaaaguugagcucaaaguagaggaaaaguuacuggugguaccuugcugccugcucugcugguagaauucugugcuccccgugacacuuaguacauuaagaaugacuacacuguuccucguaugugaaggaggcagugcugacuccgugagugugagacacgugcuuugaacugcuuuucuauucauggagcacuccauagucucaaacugucccccuuaugaccaacagcacauuugugaagagguucgcagggauaaggggugcacuuuauagcuauggaaacaugagauucuccucuauuggaagcuaauuagcccacaaaggugguaaaccuguagauugggccuuaauuagcauuguacucuaaucaaaggacucuuucuaaaccauauuuauagcuuucuuaaccuacacauagucuauacauagaugcauauuuuacccccagcuggcuagagauuuauuuguuguaaaugcuguauagauuugguuuuccuuucuuuacuuacccugguuuggauuuuuuuuuuuuuucuuuugaauggauuuaugcugucuuagcaauaugacaauaauccucuguagcuugagcuaccccuccccugcuguaacuuacgugaccugugcugucacugggcauaggacagcggcaucacgguugcauucccauuggacucaugcaccucccggaugguuuuuguuuuuuucggggguucuuugggguuuguuuguuugcuucuuuuccagaguguggaaagucuacagugcagaaaggcuugaaccugccagcugauuugaaauacuuuccccugcgcagggccguaugcauccugccaagcugcguuauauucuguacuguguacaauaaagaaguuugcuuuucguuuaccaa

SEQ ID NO: 20 - protein, 510 aa, Homo sapiens
MDGFYDQQVPFMVPGKSRSEECRGRPVIDRKRKFLDTDLAHDSEELFQDLSQLQEAWLAEAQVPDDEQFVPDFQSDNLVLHAPPPTKIKRELHSPSSELSSCSHEQALGANYGEKCLYNYCAYDRKPPSGFKPLTPPTTPLSPTHQNPLFPPPQATLPTSGHAPAAGPVQGVGPAPAPHSLPEPGPQQQTFAVPRPPHQPLQMPKMMPENQYPSEQRFQRQLSEPCHPFPPQPGVPGDNRPSYHRQMSEPIVPAAPPPPQGFKQEYHDPLYEHGVPGMPGPPAHGFQSPMGIKQEPRDYCVDSEVPNCQSSYMRGGYFSSSHEGFSYEKDPRLYFDDTCVVPERLEGKVKQEPTMYREGPPYQRRGSLQLWQFLVTLLDDPANAHFIAWTGRGMEFKLIEPEEVARRWGIQKNRPAMNYDKLSRSLRYYYEKGIMQKVAGERYVYKFVCDPDALFSMAFPDNQRPFLKAESECHLSEEDTLPLTHFEDSPAYLLDMDRCSSLPYAEGFAY